(19) United States
(12) Patent Application Publication
(10) Pub. No.: US 2024/0050432 A1
(43) Pub. Date: Feb. 15, 2024

(54) EGANELISIB FOR USE IN THE TREATMENT OF PD-L1 NEGATIVE CANCER

(71) Applicant: Infinity Pharmaceuticals, Inc., Cambridge, MA (US)

(72) Inventor: Brenda Carol O'Connell, Brighton, MA (US)

(21) Appl. No.: 18/265,915

(22) PCT Filed: Dec. 7, 2021
(86) PCT No.: PCT/US2021/062127

§ 371 Date: Jun. 7, 2023

Related U.S. Application Data
(60) Provisional application No. 63/203,515, filed on Jul. 26, 2021.
(60) Provisional application No. 63/168,123, filed on Mar. 30, 2021.
(60) Provisional application No. 63/146,470, filed on Feb. 5, 2021.
(60) Provisional application No. 63/122,892, filed on Dec. 8, 2020.

(51) Int. Cl. A61K 31/519; A61K 39/395; A61K 31/337; A61P 35/04; C07K 16/28; A61P 35/00

(52) U.S. Cl. CPC A61K 31/519; A61K 39/3955; A61K 31/337; A61P 35/04; C07K 16/2818; A61P 35/00; C07K 16/2827; A61K 2039/505

(57) ABSTRACT

Compounds and pharmaceutical compositions that modulate kinase activity, including PI3 kinase activity, and compounds, pharmaceutical compositions, and methods of treatment of diseases and conditions associated with kinase activity, including PI3 kinase activity, are described herein.

### CROSS-REFERENCE TO RELATED APPLICATIONS

This application claims priority to U.S. Provisional Application No. 63/122,892, filed on Dec. 8, 2020, 63/146,470, filed on Feb. 5, 2021, 63/168,123, filed on Mar. 30, 2021, and 63/203,515, filed on Jul. 26, 2021, the entireties of which are incorporated herein by reference.

### BACKGROUND

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases (Gaestel et al. Current Medicinal Chemistry (2007) 14:2214-2234).

Kinases represent a class of important signaling molecules. Kinases can generally be classified into protein kinases and lipid kinases, and certain kinases exhibit dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates on tyrosine residues (e.g., erb2, PDGF receptor, EGF receptor, VEGF receptor, src, abl), serine/threonine kinases which predominantly phosphorylate substrates on serine and/or threonine residues (e.g., mTorC1, mTorC2, ATM, ATR, DNA-PK, Akt), and dual-specificity kinases which phosphorylate substrates on tyrosine, serine and/or threonine residues.

Lipid kinases are enzymes that catalyze the phosphorylation of lipids. These enzymes, and the resulting phosphorylated lipids and lipid-derived biologically active organic molecules play a role in many different physiological processes, including cell proliferation, migration, adhesion, and differentiation. Certain lipid kinases are membrane associated and they catalyze the phosphorylation of lipids contained in or associated with cell membranes. Examples of such enzymes include phosphoinositide(s) kinases (e.g., PI3-kinases, PI4-kinases), diacylglycerol kinases, and sphingosine kinases.

The phosphoinositide 3-kinases (PI3Ks) signaling pathway is one of the most highly mutated systems in human cancers. PI3K signaling is also a key factor in many other diseases in humans. PI3K signaling is involved in many disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation. The class I PI3Ks (p110α, p110β, p110δ, and p110γ) are typically activated by tyrosine kinases or G-protein coupled receptors to generate PIP3, which engages downstream effectors such as those in the Akt/PDK1 pathway, mTOR, the Tec family kinases, and the Rho family GTPases. The class II and III PI3Ks play a key role in intracellular trafficking through the synthesis of PI(3)P and PI(3,4)P2. The PI3Ks are protein kinases that control cell growth (mTORC1) or monitor genomic integrity (ATM, ATR, DNA-PK, and hSmg-1).

The delta (δ) isoform of class I PI3K has been implicated, in particular, in a number of diseases and biological processes. PI3K-δ is expressed primarily in hematopoietic cells including leukocytes such as T-cells, dendritic cells, neutrophils, mast cells, B-cells, and macrophages. PI3K-δ is integrally involved in mammalian immune system functions such as T-cell function, B-cell activation, mast cell activation, dendritic cell function, and neutrophil activity. Due to its integral role in immune system function, PI3K-δ is also involved in a number of diseases related to undesirable immune response such as allergic reactions, inflammatory diseases, inflammation mediated angiogenesis, rheumatoid arthritis, and auto-immune diseases such as lupus, asthma, emphysema and other respiratory diseases. Other class I PI3K involved in immune system function includes PI3K-γ, which plays a role in leukocyte signaling and has been implicated in inflammation, rheumatoid arthritis, and autoimmune diseases such as lupus. For example, PI3K-γ and PI3K-δ are highly expressed in leukocytes and have been associated with adaptive and innate immunity; thus, these PI3K isoforms can be important mediators in inflammatory disorders and hematologic malignancies.

The gamma (γ) isoform of class I PI3K consists of a catalytic subunit p110γ, which is associated with a p101 regulatory subunit. PI3K-γ is regulated by G protein-coupled receptors (GPCRs) via association with the β/γ subunits of heterotrimeric G proteins. PI3K-γ is expressed primarily in hematopoietic cells and cardiomyocytes and is involved in inflammation, the innate immune response, myeloid cell differentiation, immune cell trafficking, and mast cell function. Inhibitors of PI3K-γ are useful for treating a variety of inflammatory diseases, allergies, and cardiovascular diseases, among others.

Binding of the PD-1 ligands, PD-L1 and PD-L2, to the PD-1 receptor found on T cells, inhibits T-cell proliferation and cytokine production. Upregulation of PD-1 ligands occurs in some tumors and signaling through this pathway can contribute to inhibition of active T-cell immune surveillance of tumors. Nivolumab is a human immunoglobulin G4 (IgG4) monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Nivolumab is a medication used to treat a number of types of cancer, including, e.g., melanoma, lung cancer, renal cell carcinoma, Hodgkin lymphoma, head and neck cancer, colon cancer, and liver cancer. Nivolumab is approved for the treatment of patients with locally advanced or metastatic urothelial carcinoma who have disease progression during or following platinum-containing chemotherapy or within 12 months of neoadjuvant or adjuvant treatment with platinum-containing chemotherapy, regardless of whether tumors express PD-L1.

While nivolumab is approved for use in advanced urothelial carcinoma, regardless of PD-L1 status, the CheckMate-275 study showed that patients with PD-L1 expression <1% had an overall response rate (ORR) of 16.4% and patients with PD-L1 expression ≥1% had an ORR of 25.8%. There remains a need for better treatment options for PD-L1 cancer such as PD-L1 negative urothelial carcinoma.

### SUMMARY

In one embodiment, provided herein is a method for treating a cancer in a subject, comprising administering to the subject a therapeutically effective amount of a compound, which is Compound 1 (also referred to herein as “eganelisib”) of the formula:

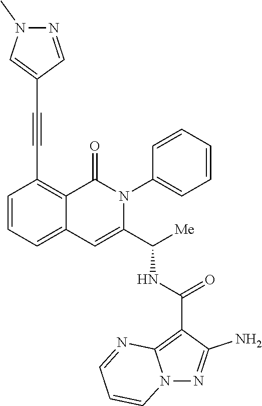 [SMILES: C[C@H](NC(=O)c1c(N)nn2cccnc12)c1cc2cccc(C#Cc3cnn(C)c3)c2c(=O)n1-c1ccccc1]

or an enantiomer, a mixture of enantiomers, or a mixture of two or more diastereomers thereof, or a pharmaceutically acceptable form thereof, and wherein the cancer is PD-L1 negative. In some embodiments, Compound 1 is a PI3K gamma inhibitor, e.g., a selective PI3K-gamma inhibitor.

In one embodiment, the method further comprises administering to the subject a therapeutically effective amount of a second agent. In one embodiment, the immune checkpoint therapy is a PD-L1 inhibitor. In one embodiment, the immune checkpoint therapy is a PD-1 inhibitor.

In one embodiment, the PD-L1 negative cancer is breast cancer, renal cell carcinoma, or urothelial carcinoma.

In one embodiment, provided herein is a method for treating a PD-L1 negative patient with immune therapy-naïve, advanced urothelial carcinoma, comprising administering to the patient a therapeutically effective amount of Compound 1, or a pharmaceutically acceptable salt thereof, in combination with nivolumab.

In one embodiment, provided herein is a kit comprising a compound described herein, for use in a method provided herein.

In one embodiment, provided herein is use of a compound or a pharmaceutical composition described herein for the treatment of a disease or disorder described herein in a subject. In one embodiment, provided herein is use of a compound or a pharmaceutical composition described herein for the treatment of a PI3K mediated disorder described herein in a subject. In one embodiment, provided herein is use of a compound or a pharmaceutical composition described herein in the manufacture of a medicament for the treatment of a disease or disorder described herein in a subject. In one embodiment, provided herein is use of a compound or a pharmaceutical composition described herein in the manufacture of a medicament for the treatment of a PI3K mediated disorder described herein in a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In case of conflict, the present application, including any definitions herein, will control.

### BRIEF DESCRIPTION OF THE DRAWINGS

FIG. 1 shows eganelisib (Compound 1) mechanism of action.

FIG. 2 shows scientific rationale for adding eganelisib to atezo and nab-Pac in 1 L TNBC.

FIG. 3 shows phase II study design: triple combination to improve approved 1 L TNBC regimen.

FIG. 4 shows clinical response: 100% of evaluable patients exhibited tumor reduction with 9/13 (69.2%) exhibiting a complete or partial response regardless of PD-L1 status.

FIG. 5 shows peripheral blood analyses support mechanism of action.

FIG. 6 shows PD-L1 negative TNBC patient B: significant tumor reduction in macrophage rich tumor.

FIG. 7 shows phase II study design to evaluate addition of eganelisib to standard of care (nivolumab) in I/O Naïve UC Patients.

FIG. 8 shows best percent change in tumor volume of target lesion (N=40).

FIG. 9 shows preliminary progression free survival results.

FIG. 10 shows increased immune activation for eganelisib+nivolumab vs. nivolumab across PD-L1 negative and PD-L1 positive patients.

FIG. 11A shows preliminary overall survival results in all patients; FIG. 11B shows preliminary overall survival results in PD-L1 negative patients.

FIG. 12A and FIG. 12B show stable disease contribution to overall survival.

FIG. 13 shows comparison of median Overall Survival (mOS) for eganelisib plus nivolumab in 2 L urothelial carcinoma patients to benchmark 2 L studies.

FIG. 14 shows comparison of median Overall Survival (mOS) for eganelisib plus nivolumab in 2 L to checkpoint inhibitors (CPIs) in 1 L mUC patients.

FIG. 15 shows that 86.8% of evaluable TNBC patients receiving eganelisib plus atezo and nab-Pac achieved tumor reduction.

FIG. 16 shows mPFS data for eganelisib+atezolizumab+nab-paclitaxel in both PD-L1(+) and PD-L1(−) patients.

FIG. 17 shows durable clinical benefit in patients with SD as well as those with PRs and CRs.

FIG. 18A and FIG. 18B show reduced immune suppression and increased immune activation regardless of PD-L1 status.

FIG. 19 shows on-mechanism conversion of patients from PD-L1(−) to PD-L1(+) and increase in PD-L1 expression in PD-L1(+) patients.

FIG. 20 shows that 88.6% of evaluable TNBC patients receiving eganelisib plus atezo and nab-Pac achieved tumor reduction.

FIG. 21 shows durable clinical benefit in patients regardless of baseline PD-L1.

FIG. 22 shows increased immune activation regardless of baseline PD-L1 status in peripheral blood.

### DETAILED DESCRIPTION

### Definitions

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, “agent” or “biologically active agent” or “second active agent” refers to a biological, pharmaceutical, or chemical compound or other moiety. Non-limiting examples include simple or complex organic or inorganic molecules, a peptide, a protein, an oligonucleotide, an antibody, an antibody derivative, an antibody fragment, a vitamin, a vitamin derivative, a carbohydrate, a toxin, or a chemotherapeutic compound, and metabolites thereof. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of this disclosure.

The term “agonist” as used herein refers to a compound or agent having the ability to initiate or enhance a biological function of a target protein or polypeptide, such as increasing the activity or expression of the target protein or polypeptide. Accordingly, the term “agonist” is defined in the context of the biological role of the target protein or polypeptide. While some agonists herein specifically interact with (e.g., bind to) the target, compounds and/or agents that initiate or enhance a biological activity of the target protein or polypeptide by interacting with other members of the signal transduction pathway of which the target polypeptide is a member are also specifically included within this definition.

The terms “antagonist” and “inhibitor” are used interchangeably, and they refer to a compound or agent having the ability to inhibit a biological function of a target protein or polypeptide, such as by inhibiting the activity or expression of the target protein or polypeptide. Accordingly, the terms “antagonist” and “inhibitor” are defined in the context of the biological role of the target protein or polypeptide. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease. The term “inhibition” or “inhibitor” as used in this context includes a reduction in a certain parameter, e.g., an activity, of a given molecule, e.g., a PI3K isoform. For example, inhibition of an activity, e.g., a PI3K activity, of at least 5%, 10%, 20%, 30%, 40% or more is included by this term. Thus, inhibition need not be 100%.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, or buccal administration, or inhalation, or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “tumor” refers to any neoplastic cell growth and proliferation, whether malignant or benign, and any pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

Hematopoietic origin refers to involving cells generated during hematopoiesis, a process by which cellular elements of blood, such as lymphocytes, leukocytes, platelets, erythrocytes and natural killer cells are generated. Cancers of hematopoietic origin includes lymphoma and leukemia.

“Resistant” or “refractory” or “refractive” refers to when a cancer that has a reduced responsiveness to a treatment, e.g., up to the point where the cancer does not respond to treatment. The cancer can be resistant at the beginning of treatment, or it may become resistant during treatment. The cancer subject can have one or more mutations that cause it to become resistant to the treatment, or the subject may have developed such mutations during treatment. In one embodiment, the cancer or subject has failed to respond to a given therapeutic treatment (e.g., has failed to respond by at least 10%, 20%, 30%, 40%, 50%, 60%, 70%, 80%, 90%, or 100% to a given treatment). Failed treatment can be measured by, e.g., tumor volume or the length of time before tumor regrowth occurs.

By “hyperproliferative cancerous disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growth, benign tumors, malignant tumors, and “cancer.”

Combination therapy or “in combination with” refer to the use of more than one compound or agent to treat a particular disorder or condition. For example, Compound 1 may be administered in combination with at least one additional therapeutic agent. By “in combination with,” it is not intended to imply that the other therapy and Compound 1 must be administered at the same time and/or formulated for delivery together, although these methods of delivery are within the scope of this disclosure. Compound 1 can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. In general, each therapeutic agent will be administered at a dose and/or on a time schedule determined for that particular agent. The other therapeutic agent can be administered with Compound 1 herein in a single composition or separately in a different composition. Higher combinations, e.g., triple therapy, are also contemplated herein.

As used herein, a “monotherapy” refers to the use of an agent individually (e.g., as a single compound or agent), e.g., without a second active ingredient to treat the same indication, e.g., cancer. For example, in this context, the term monotherapy includes the use of either the PI3K inhibitor or the second agent individually to treat the cancer.

The term “synergy” or “synergistic” encompasses a more than additive effect of a combination of two or more agents compared to their individual effects. In certain embodiments, synergy or synergistic effect refers to an advantageous effect of using two or more agents in combination, e.g., in a pharmaceutical composition, or in a method of treatment. In certain embodiments, one or more advantageous effects is achieved by using a PI3K inhibitor in combination with a second therapeutic agent (e.g., one or more second therapeutic agents) as described herein.

In embodiments, the synergistic effect is that a lower dosage of one or both of the agents is needed to achieve an effect. For example, the combination can provide a selected effect, e.g., a therapeutic effect, when at least one of the agents is administered at a lower dosage than the dose of that agent that would be required to achieve the same therapeutic effect when the agent is administered as a monotherapy. In certain embodiments, the combination of a PI3K inhibitor (e.g., Compound 1) and a second agent (as described herein) allows the PI3K inhibitor to be administered at a lower dosage than would be required to achieve the same therapeutic effect if the PI3K inhibitor were administered as a monotherapy.

In embodiments, a synergistic effect refers to the combination of a PI3K inhibitor (e.g., Compound 1, or a pharmaceutically acceptable form thereof), and a second therapeutic agent (e.g., one or more additional therapeutic agent(s), or a pharmaceutically acceptable form thereof, as described herein), results in a therapeutic effect greater than the additive effect of the PI3K inhibitor and the second agent.

In embodiments, a synergistic effect means that combination index value is less than a selected value, e.g., for a given effect, e.g., at a selected percentage (e.g., 50%) inhibition or growth inhibition, e.g., as described herein in the Examples. In embodiments, a synergistic effect means that the synergy score is 1 or more. In certain embodiments, the synergy score is greater than 1. In certain embodiments, the synergy score is greater than 3.

Combination index (CI) is a measure of potency shifting. The combination index is known in the art and is described, e.g., in Chou et al., Adv Enzyme Regul 1984; 22: 27-55 and inL U.S. Patent Publication No. 2013/0295102, the contents of which are incorporated herein by reference. A CI value of greater than 1 indicates antagonistic effect; a CI value of 1.0 is indicative of an additive effect; and a CI value of less than 1 is indicative of a synergistic effect resulting from the combination. The CI value can be determined at various percentages of inhibition or growth inhibition.

The CI provides an estimate of the fraction of the original (monotherapy) doses of each of two drugs would be needed in combination relative to the single agent doses required to achieve a chosen effect level. For example, when the combination index has a value of 0.1, only about one tenth of the total fractional amounts of the individual agents (expressed as a fraction of the amount of that agent when administered as a monotherapy to achieve a chosen effect) are needed for the combination to reach the same chosen effect level. For example, if a dose of 100 mg/kg of drug A individually or a dose of 200 mg/kg of drug B individually is needed to achieve the chosen effect, and the combination index is 0.1, then approximately 5 mg/kg of drug A and 10 mg/kg of drug B would achieve the chosen effect (one twentieth of the original doses of each of the single agents adds up to a total of one tenth). The doses of the single agents need not be reduced by the same fractional value so long as the sum of their fractional values adds up to the combination index; thus, in this example, a dose of approximately 8 mg/kg of drug A and 4 mg/kg of drug B would also achieve the chosen effect (this is 0.08 times the original dose of drug A and 0.02 times the original dose of drug B; the sum of the fractional amounts (0.08+0.02) is equal to the combination index of 0.1.)

According to one embodiment, synergy score is a measure of the combination effects in excess of Loewe additivity. In one example, synergy score is a scalar measure to characterize the strength of synergistic interaction. The Synergy score can be calculated as:

Synergy Score=log fₓ log f_{y} Σ max(0,I_{data})(I_{data}−I_{Loewe})

In this example, the fractional inhibition for each component agent and combination point in the matrix is calculated relative to the median of all vehicle-treated control wells. The example Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels. According to other embodiments, a synergy score can be calculated based on a curve fitting approach where the curvature of the synergy score is extrapolated by introducing a median value and origin value (e.g., a dose zero value).

The synergy score measure can be used for the self-cross analysis. Synergy scores of self-crosses are expected to be additive by definition and, therefore, maintain a synergy score of zero. However, while some self-cross synergy scores are near zero, many are greater suggesting that experimental noise or non-optimal curve fitting of the single agent dose responses are contributing to the slight perturbations in the score. This strategy is cell line-centric, focusing on self-cross behavior in each cell line versus a global review of cell line panel activity. Combinations where the synergy score is greater than the mean self-cross plus two standard deviations or three standard deviations can be considered candidate synergies at 95% and 99% confidence levels, respectively. Additivity should maintain a synergy score of zero, and synergy score of two or three standard deviations indicate synergism at statistically significant levels of 95% and 99%.

Loewe Volume (Loewe Vol) can be used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies (X/X₁)+(Y/Y₁)=1

where X₁ and Y₁ are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the terms “treatment”, “treating”, “palliating” and “ameliorating” are used interchangeably herein. These terms refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, the terms “prevention” and “preventing” are used herein to refer to an approach for obtaining beneficial or desired results including, but not limited, to prophylactic benefit. For prophylactic benefit, the pharmaceutical compositions can be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

“Signal transduction” or “signaling pathway” is a process during which stimulatory or inhibitory signals are transmitted into and within a cell to elicit an intracellular response. A “modulator” of a signal transduction pathway refers to a compound which modulates the activity of one or more cellular proteins mapped to the same specific signal transduction pathway. A modulator can augment (agonist) or suppress or inhibit (antagonist) the activity of a signaling molecule.

In certain embodiments, the signal transduction is mediated by one or more phosphoinositide 3-kinases (PI3Ks). PI3Ks are members of a conserved family of lipid kinases that regulate numerous cell functions, including proliferation, differentiation, cell survival and metabolism. Several classes of PI3Ks exist in mammalian cells, including Class IA subgroup (e.g., PI3K-α, β, δ), which are generally activated by receptor tyrosine kinases (RTKs); Class IB (e.g., PI3K-γ), which is activated by G-protein coupled receptors (GPCRs), among others. PI3Ks exert their biological activities via a “PI3K-mediated signaling pathway” that includes several components that directly and/or indirectly transduce a signal triggered by a PI3K, including the generation of second messenger phophotidylinositol, 3,4,5-triphosphate (PIP3) at the plasma membrane, activation of heterotrimeric G protein signaling, and generation of further second messengers such as cAMP, DAG, and IP3, all of which leads to an extensive cascade of protein kinase activation (reviewed in Vanhaesebroeck, B. et al. (2001) Annu Rev Biochem. 70:535-602). In certain embodiments, the compounds disclosed herein inhibit a PI3 kinase or PI3K) isoform, e.g., one, two, three or more of PI3K-α, β, δ or -γ.

In the context of biological molecules, to “decrease”, “suppress,” “ameliorate,” “reduce,” “inhibit,” or the like, includes decreasing a level or an activity (e.g., one or more functions) of a given molecule. The level of a given molecule, e.g., mRNA or protein level, or the activity can be measured in a sample, or using the assays described in the Examples herein.

To “decrease,” “ameliorate,” “reduce,” “inhibit,” (or the like) a disorder or condition, or a symptom associated with a disorder or condition includes reducing the severity and/or frequency of one or more symptoms of the disorder or condition, or reducing or delaying the onset of the disorder or condition and/or one or more symptoms of the disorder or condition. In some embodiments, the symptom is reduced by at least about 2%, at least about 5%, at least about 10%, at least about 15%, at least about 20%, at least about 25%, at least about 30%, at least about 40%, at least about 50%, at least about 60%, at least about 70%, at least about 80%, at least about 90%, or at least about 95% relative to a control level.

The term “inhibition” or “inhibit” as used in this context includes a reduction in a certain parameter, e.g., an activity, of a given molecule, e.g., a PI3K isoform. For example, inhibition of an activity, e.g., a PI3K activity, of at least 5%, 10%, 20%, 30%, 40% or more is included by this term. Thus, inhibition need not be 100%. In certain embodiments, a PI3K inhibitor as disclosed herein inhibits a PI3 kinase of the gamma isoform (a “PI3K-γ isoform).

The term “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits one isoform of PI3K over another isoform of PI3K has an activity of at least greater than about 1× against a first isoform relative to the compound's activity against the second isoform (e.g., at least about 2×, 3×, 5×, 10×, 20×, 50×, 100×, 200×, 500×, or 1000×).

As used herein, a “reference value” refers to a value to which a given value can be compared. In some embodiments, the reference value refers to a control (e.g., an untreated control, e.g., an untreated or placebo-treated subject or an untreated sample); the course of disease without treatment; a healthy subject or an average of healthy subjects; a subject at a different time interval, e.g., prior to, during, or after the treatment).

“Subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In one embodiment, the subject is a human patient.

The term “in vivo” refers to an event that takes place in a subject's body.

The term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In one embodiment, a “pharmaceutically acceptable form” includes, but is not limited to, pharmaceutically acceptable salts, isomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, naphthalene-m,n-bissulfonates, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, naphthalene-m,n-bissulfonic acids and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is an isomer. “Isomers” are different compounds that have the same molecular formula. “Atropisomers” are stereoisomers from hindered rotation about single bonds and can be resolved or isolated by methods known to those skilled in the art. For example, certain B substituents of a compound of Formula (I) provided herein with ortho or meta substituted phenyl may form atropisomers, where they may be separated and isolated.

“Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof, and other mixtures thereof, as falling within the scope of this disclosure.

In certain embodiments, provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of the plane of the ring are designated “cis trans.”

“Enantiomers” are a pair of stereoisomers that are non-superimposable mirror images of each other. A mixture of a pair of enantiomers in any proportion can be known as a “racemic” mixture. The term “(+)” is used to designate a racemic mixture where appropriate. “Diastereoisomers” are stereoisomers that have at least two asymmetric atoms, but which are not mirror-images of each other. The absolute stereochemistry can be specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

The “enantiomeric excess” or “% enantiomeric excess” of a composition can be calculated using the equation shown below. In the example shown below, a composition contains 90% of one enantiomer, e.g., an S enantiomer, and 10% of the other enantiomer, e.g., an R enantiomer.

ee=(90−10)/100=80%.

Thus, a composition containing 90% of one enantiomer and 10% of the other enantiomer is said to have an enantiomeric excess of 80%. Some compositions described herein contain an enantiomeric excess of at least about 1%, about 5%, about 10%, about 20%, about 30%, about 40%, about 50%, about 75%, about 90%, about 95%, or about 99% of the S enantiomer. In other words, the compositions contain an enantiomeric excess of the S enantiomer over the R enantiomer. In other embodiments, some compositions described herein contain an enantiomeric excess of at least about 1%, about 5%, about 10%, about 20%, about 30%, about 40%, about 50%, about 75%, about 90%, about 95%, or about 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the amount of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the total weight of the preparation (e.g., total weight of S and R isomers). such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to the total weight of the preparation, such as at least about 90% by weight, and further such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, in some embodiments, the compound mixture has an (S)-enantiomeric excess of greater than about 10%, greater than about 20%, greater than about 30%, greater than about 40%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, the compound mixture has an (S)-enantiomeric excess of about 55%, about 60%, about 65%, about 70%, about 75%, about 80%, about 85%, about 90%, about 95%, about 96%, about 97%, about 98%, about 99%, or about 99.5%, or more. In some embodiments, the compound mixture has an (S)-enantiomeric excess of about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

Enantiomers can be isolated from racemic mixtures by any method known to those skilled in the art, including chiral high pressure liquid chromatography (HPLC), the formation and crystallization of chiral salts, or prepared by asymmetric syntheses. See, for example, Enantiomers, Racemates and Resolutions (Jacques, Ed., Wiley Interscience, New York, 1981); Wilen et al., Tetrahedron 33:2725 (1977); Stereochemistry of Carbon Compounds (E. L. Eliel, Ed., McGraw-Hill, NY, 1962); and Tables of Resolving Agents and Optical Resolutions p. 268 (E. L. Eliel, Ed., Univ. of Notre Dame Press, Notre Dame, IN 1972).

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions.

### Compounds

In one embodiment, the compound used in the methods provided herein is Compound 1 of the structure:

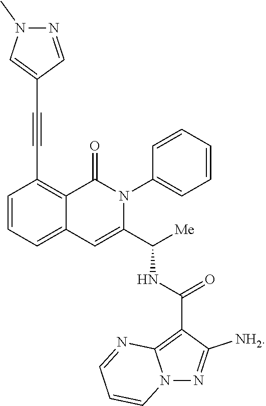 [SMILES: C[C@H](NC(=O)c1c(N)nn2cccnc12)c1cc2cccc(C#Cc3cnn(C)c3)c2c(=O)n1-c1ccccc1]

Compound 1 is also called eganelisib, and is a PI3K-gamma inhibitor. The synthesis and biological activities of the compound is described in WO 2015/051244; certain medical uses, polymorphic forms, and synthetic processes for Compound 1 are described in WO 2015/143012 and WO 2017/048702; the entirety of each of which is incorporated herein by reference.

### Method of Uses

In one embodiment, provided herein is a method for treating a cancer in a subject, comprising administering to the subject a therapeutically effective amount of a compound of the formula:

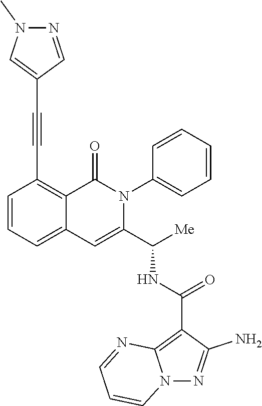 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, and wherein the cancer is PD-L1 negative.

In one embodiment, provided herein is a method for treating a cancer in a subject, comprising: (i) identifying the cancer in the subject to be PD-L1 negative, and (ii) administering to the subject a therapeutically effective amount of a compound of the formula:

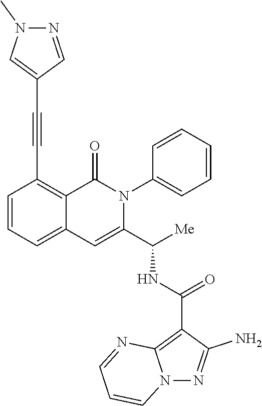 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof.

In one embodiment, the (PD-L1 negative) cancer is selected from one or more of: a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an esophageal cancer, a hepatocellular cancer, a gastric cancer, a gastrointestinal stromal tumor (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumor, diffuse type giant cell tumor, and glioblastoma.

In one embodiment, the cancer is a solid tumor. In one embodiment, the solid tumor is not breast cancer or renal cell carcinoma.

In one embodiment, the solid tumor is melanoma, lung cancer, head and neck cancer, renal cell carcinoma, gallbladder carcinoma, breast cancer, colon cancer, glioblastoma, adrenocortical carcinoma, mesothelioma, colorectal cancer, ovarian cancer, endometrial cancer, or urothelial carcinoma.

In one embodiment, the solid tumor patient is naive to immune therapy (e.g., immune checkpoint therapy, e.g., anti-PD-1 and/or anti-PD-L1 therapy). In one embodiment, the solid tumor patient has been previously treated with one or more (e.g., two or more) immune therapy (e.g., immune checkpoint therapy, e.g., anti-PD-1 and/or anti-PD-L1 therapy).

In one embodiment, the solid tumor is breast cancer. In one embodiment, the breast cancer is triple negative breast cancer. In one embodiment, the breast cancer is the breast cancer is unresectable locally advanced or metastatic triple negative breast cancer. In one embodiment, the breast cancer (e.g., triple negative breast cancer) patient is naive to immune therapy (e.g., immune checkpoint therapy, e.g., anti-PD-1 and/or anti-PD-L1 therapy). In one embodiment, the breast cancer (e.g., triple negative breast cancer) patient has been previously treated with one or more (e.g., two or more) immune therapy (e.g., immune checkpoint therapy, e.g., anti-PD-1 and/or anti-PD-L1 therapy).

In one embodiment, the solid tumor is head and neck cancer. In one embodiment, the head and neck cancer is head and neck squamous cell carcinoma. In one embodiment, the head and neck cancer (e.g., head and neck squamous cell carcinoma) patient is naive to immune therapy (e.g., immune checkpoint therapy, e.g., anti-PD-1 and/or anti-PD-L1 therapy). In one embodiment, the head and neck cancer (e.g., head and neck squamous cell carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is lung cancer. In one embodiment, the lung cancer is non-small cell lung cancer. In one embodiment, the lung cancer (e.g., non-small cell lung cancer) patient is naive to immune therapy (e.g., immune checkpoint therapy, e.g., anti-PD-1 and/or anti-PD-L1 therapy). In one embodiment, the lung cancer (e.g., non-small cell lung cancer) patient has been previously treated with one or more (e.g., two or more) immune therapy (e.g., immune checkpoint therapy, e.g., anti-PD-1 and/or anti-PD-L1 therapy).

In one embodiment, the solid tumor is melanoma. In one embodiment, the melanoma patient is naive to immune therapy (e.g., immune checkpoint therapy, e.g., anti-PD-1 and/or anti-PD-L1 therapy). In one embodiment, the melanoma patient has been previously treated with one or more (e.g., two or more) immune therapy (e.g., immune checkpoint therapy, e.g., anti-PD-1 and/or anti-PD-L1 therapy).

In one embodiment, the solid tumor is colon cancer. In one embodiment, the colon cancer patient is naive to immune therapy (e.g., immune checkpoint therapy, e.g., anti-PD-1 and/or anti-PD-L1 therapy). In one embodiment, the colon cancer patient has been previously treated with one or more (e.g., two or more) immune therapy (e.g., immune checkpoint therapy, e.g., anti-PD-1 and/or anti-PD-L1 therapy).

In one embodiment, the solid tumor is glioblastoma. In one embodiment, the glioblastoma patient is naive to immune therapy (e.g., immune checkpoint therapy, e.g., anti-PD-1 and/or anti-PD-L1 therapy). In one embodiment, the glioblastoma patient has been previously treated with one or more (e.g., two or more) immune therapy (e.g., immune checkpoint therapy, e.g., anti-PD-1 and/or anti-PD-L1 therapy).

In one embodiment, the solid tumor is renal cell carcinoma. In one embodiment, the renal cell carcinoma is clear cell renal cell carcinoma. In one embodiment, the renal cell carcinoma (e.g., clear cell renal cell carcinoma) patient is naive to immune therapy (e.g., immune checkpoint therapy, e.g., anti-PD-1 and/or anti-PD-L1 therapy). In one embodiment, the renal cell carcinoma (e.g., clear cell renal cell carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is gallbladder carcinoma. In one embodiment, the gallbladder carcinoma is microsatellite-stable gallbladder carcinoma. In one embodiment, the gallbladder carcinoma (e.g., microsatellite-stable gallbladder carcinoma) patient is naive to immune therapy (e.g., immune checkpoint therapy, e.g., anti-PD-1 and/or anti-PD-L1 therapy). In one embodiment, the gallbladder carcinoma (e.g., microsatellite-stable gallbladder carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is adrenocortical carcinoma. In one embodiment, the adrenocortical carcinoma patient is naive to immune therapy (e.g., immune checkpoint therapy, e.g., anti-PD-1 and/or anti-PD-L1 therapy). In one embodiment, the adrenocortical carcinoma patient has been previously treated with one or more (e.g., two or more) immune therapy (e.g., immune checkpoint therapy, e.g., anti-PD-1 and/or anti-PD-L1 therapy).

In one embodiment, the solid tumor is mesothelioma. In one embodiment, the mesothelioma is epithelioid mesothelioma, sarcomatoid mesothelioma, or biphasic mesothelioma. In one embodiment, the mesothelioma patient is naive to immune therapy (e.g., immune checkpoint therapy, e.g., anti-PD-1 and/or anti-PD-L1 therapy). In one embodiment, the mesothelioma patient has been previously treated with one or more (e.g., two or more) immune therapy (e.g., immune checkpoint therapy, e.g., anti-PD-1 and/or anti-PD-L1 therapy).

In one embodiment, the solid tumor is colorectal cancer. In one embodiment, the colorectal cancer patient is naive to immune therapy (e.g., immune checkpoint therapy, e.g., anti-PD-1 and/or anti-PD-L1 therapy). In one embodiment, the colorectal cancer patient has been previously treated with one or more (e.g., two or more) immune therapy (e.g., immune checkpoint therapy, e.g., anti-PD-1 and/or anti-PD-L1 therapy).

In one embodiment, the solid tumor is ovarian cancer. In one embodiment, the ovarian cancer patient is naive to immune therapy (e.g., immune checkpoint therapy, e.g., anti-PD-1 and/or anti-PD-L1 therapy). In one embodiment, the ovarian cancer patient has been previously treated with one or more (e.g., two or more) immune therapy (e.g., immune checkpoint therapy, e.g., anti-PD-1 and/or anti-PD-L1 therapy).

In one embodiment, the solid tumor is endometrial cancer. In one embodiment, the endometrial cancer patient is naive to immune therapy (e.g., immune checkpoint therapy, e.g., anti-PD-1 and/or anti-PD-L1 therapy). In one embodiment, the endometrial cancer patient has been previously treated with one or more (e.g., two or more) immune therapy (e.g., immune checkpoint therapy, e.g., anti-PD-1 and/or anti-PD-L1 therapy).

In one embodiment, the solid tumor is urothelial carcinoma. In one embodiment, the urothelial carcinoma patient is naive to immune therapy (e.g., immune checkpoint therapy, e.g., anti-PD-1 and/or anti-PD-L1 therapy). In one embodiment, the urothelial carcinoma patient has been previously treated with one or more (e.g., two or more) immune therapy (e.g., immune checkpoint therapy, e.g., anti-PD-1 and/or anti-PD-L1 therapy).

In one embodiment, the cancer is a hematological cancer.

In one embodiment, the hematological cancer is leukemia or lymphoma.

In one embodiment, the hematological cancer is acute lymphocytic leukemia (ALL), chronic lymphocytic leukemia (CLL), prolymphocytic leukemia (PLL), hairy cell leukemia (HLL), Waldenstrom's macroglobulinemia (WM), peripheral T cell lymphomas (PTCL), adult T cell leukemia/lymphoma (ATLL), cutaneous T-cell lymphoma (CTCL), large granular lymphocytic leukemia (LGL), acute myelocytic leukemia (AML), Hodgkin lymphoma (HL), non-Hodgkin lymphoma (NHL), follicular lymphoma, diffuse large B-cell lymphoma (DLBCL), mantle cell lymphoma (MCL), mastocytosis, multiple myeloma (MM), myelodysplastic syndrome (MDS), or myeloproliferative disorder (MPD).

In some embodiments, the cancer is a solid or soft tissue tumor (e.g., a carcinoid, carcinoma or sarcoma), a hematopoietic tissue tumor (e.g., a heme malignancy), or a metastatic lesion, e.g., a metastatic lesion of any of the cancers or tumors disclosed herein. In one embodiment, the cancer is metastatic cancer to the bone.

In one embodiment, the (PD-L1 negative) cancer treated by the methods or compounds disclosed herein is a soft tissue tumor, a heme malignancy, or a hematological cancer. In one embodiment, the cancer is acute myeloid leukemia (AML), chronic myeloid leukemia (CML), myelodysplastic syndrome (MDS), myeloproliferative disorders, mast cell cancer, Hodgkin disease, non-Hodgkin lymphomas, diffuse large B-cell lymphoma, human lymphotrophic virus type 1 (HTLV-1) leukemia/lymphoma, AIDS-related lymphoma, adult T-cell lymphoma, acute lymphoblastic leukemia (ALL), T-cell acute lymphoblastic leukemia, B-cell acute lymphoblastic leukemia, chronic lymphocytic leukemia, or multiple myeloma (MM). In one embodiment, the cancer is leukemia or lymphoma. In one embodiment, the leukemia is B-cell acute lymphoblastic leukemia (B-ALL), acute myeloid leukemia (AML), acute lymphoblastic leukemia, chronic myeloid leukemia, hairy cell leukemia, myeloproliferative disorders, acute myelogenous leukemia (AML), chronic myelogenous leukemia (CML), chronic lymphocytic leukemia (CLL), multiple myeloma (MM), myelodysplastic syndrome (MDS), or mast cell cancer. In one embodiment, the lymphoma is diffuse large B-cell lymphoma, B-cell immunoblastic lymphoma, small non-cleaved cell or Burkitt lymphoma, human lymphotropic virus-type 1 (HTLV-1) leukemia/lymphoma, adult T-cell lymphoma, Hodgkin disease, or non-Hodgkin lymphomas, or a metastatic lesion thereof.

In one embodiment, the (PD-L1 negative) cancer treated by the methods or compounds disclosed herein is a solid tumor (e.g., a carcinoid, carcinoma or sarcoma), or a metastatic lesion thereof. In one embodiment, the cancer is a lung cancer (e.g., non-small cell lung cancer or small cell lung cancer); a skin cancer; a melanoma; a prostate cancer; a glioblastoma; an endometrial cancer; a pancreatic cancer (e.g., pancreatic adenocarcinoma (e.g., pancreatic ductal adenocarcinoma (PDA)); a renal cell carcinoma; a colorectal cancer; a breast cancer (e.g., triple negative breast cancer); a thyroid cancer; a sarcoma, a liver or hepatocellular cancer (HCC), a head and neck cancer, a cervical or vulvar cancer, an esophageal cancer, a gastric cancer, an adrenal cancer, or an ovarian cancer, or a metastatic lesion thereof. In one embodiment, the solid tumor is prostate cancer, breast cancer, or a glioblastoma, or a metastatic lesion thereof.

In some embodiments, the (PD-L1 negative) cancer or tumor treated is a solid, fibrotic tumor chosen from one or more of pancreatic (e.g., pancreatic adenocarcinoma or pancreatic ductal adenocarcinoma), breast, colorectal, colon, lung (e.g., a small or non-small cell lung cancer), skin, ovarian, prostate, cervix, gastrointestinal (e.g., carcinoid or stromal), stomach, head and neck, kidney, brain cancer, or a metastatic lesion thereof.

In some embodiments, the (PD-L1 negative) cancer or tumor treated using the methods or compounds disclosed herein is a cancer or tumor chosen from one or more of the head, neck, nasal cavity, paranasal sinuses, nasopharynx, oral cavity, oropharynx, larynx, hypopharynx, salivary glands, paragangliomas, pancreas, stomach, skin, esophagus, endometrium, liver and biliary tree, bone, intestine, colon, rectum, ovaries, prostate, lung, breast, lymphatic system, blood, bone marrow central nervous system, brain, or a metastatic lesion thereof.

In one embodiment, the (PD-L1 negative) cancer is locally advanced and/or metastatic. In one embodiment, the cancer is advanced. In one embodiment, the cancer is locally advanced. In one embodiment, the cancer is metastatic.

Phosphoinositide 3-kinases (PI3Ks) are members of a conserved family of lipid kinases that regulate numerous cell functions, including proliferation, differentiation, cell survival and metabolism. Several classes of PI3Ks exist in mammalian cells, including Class IA subgroup (e.g., PI3K-α, β, δ), which are generally activated by receptor tyrosine kinases (RTKs); Class IB (e.g., PI3K-γ), which is activated by G-protein coupled receptors (GPCRs), among others. PI3Ks exert their biological activities via a “PI3K-mediated signaling pathway” that includes several components that directly and/or indirectly transduce a signal triggered by a PI3K, including the generation of second messenger phophotidylinositol, 3,4,5-triphosphate (PIP3) at the plasma membrane, activation of heterotrimeric G protein signaling, and generation of further second messengers such as cAMP, DAG, and IP3, all of which leads to an extensive cascade of protein kinase activation (reviewed in Vanhaesebroeck, B. et al. (2001) Annu Rev Biochem. 70:535-602). For example, PI3K-δ is activated by cellular receptors through interaction between the PI3K regulatory subunit (p85) SH2 domains, or through direct interaction with RAS. PIP3 produced by PI3K activates effector pathways downstream through interaction with plextrin homology (PH) domain containing enzymes (e.g., PDK-1 and AKT [PKB]). (Fung-Leung W P. (2011) Cell Signal. 23(4):603-8). Unlike PI3K-δ, PI3K-γ is not associated with a regulatory subunit of the p85 family, but rather with a regulatory subunit in the p101 or p84 families. PI3K-γ is associated with GPCRs, and is responsible for the very rapid induction of PIP3. PI3K-γ can be also activated by RAS.

In some embodiments, provided herein are methods of modulating a PI3 kinase activity (e.g., selectively modulating) by contacting the kinase with an effective amount of a compound as provided herein, or a pharmaceutically acceptable form (e.g., pharmaceutically acceptable salts, hydrates, solvates, isomers, prodrugs, and isotopically labeled derivatives) thereof, or a pharmaceutical composition as provided herein. Modulation can be inhibition (e.g., reduction) or activation (e.g., enhancement) of kinase activity. In some embodiments, provided herein are methods of inhibiting kinase activity by contacting the kinase with an effective amount of a compound as provided herein in solution. In some embodiments, provided herein are methods of inhibiting the kinase activity by contacting a cell, tissue, organ that express the kinase of interest, with a compound provided herein. In some embodiments, provided herein are methods of inhibiting kinase activity in a subject by administering into the subject an effective amount of a compound as provided herein, or a pharmaceutically acceptable form thereof. In some embodiments, the kinase activity is inhibited (e.g., reduced) by more than about 25%, 30%, 40%, 50%, 60%, 70%, 80%, or 90%, when contacted with a compound provided herein as compared to the kinase activity without such contact. In some embodiments, provided herein are methods of inhibiting PI3 kinase activity in a subject (including mammals such as humans) by contacting said subject with an amount of a compound as provided herein sufficient to inhibit or reduce the activity of the PI3 kinase in said subject.

In some embodiments, the kinase is a lipid kinase or a protein kinase. In some embodiments, the kinase is selected from a PI3 kinase including different isoforms, such as PI3 kinase α, PI3 kinase β, PI3 kinase γ, PI3 kinase δ; DNA-PK; mTOR; Abl, VEGFR, Ephrin receptor B4 (EphB4); TEK receptor tyrosine kinase (TIE2); FMS-related tyrosine kinase 3 (FLT-3); Platelet derived growth factor receptor (PDGFR); RET; ATM; ATR; hSmg-1; Hck; Src; Epidermal growth factor receptor (EGFR); KIT; Insulin Receptor (IR); and IGFR.

As used herein, a “PI3K-mediated disorder” refers to a disease or condition involving aberrant PI3K-mediated signaling pathway. In one embodiment, provided herein is a method of treating a PI3K mediated disorder in a subject, the method comprising administering a therapeutically effective amount of a compound as provided herein, or a pharmaceutically acceptable form thereof, or a pharmaceutical composition as provided herein. In some embodiments, provided herein is a method of treating a PI3K-δ or PI3K-γ mediated disorder in a subject, the method comprising administering a therapeutically effective amount of a compound as provided herein, or a pharmaceutically acceptable form thereof, or a pharmaceutical composition as provided herein. In some embodiments, provided herein is a method for inhibiting at least one of PI3K-δ and PI3K-γ, the method comprising contacting a cell expressing PI3K in vitro or in vivo with an effective amount of a compound or composition provided herein. PI3Ks have been associated with a wide range of conditions, including immunity, cancer and thrombosis (reviewed in Vanhaesebroeck, B. et al. (2010) Current Topics in Microbiology and Immunology, DOI 10.1007/82_2010_65). For example, Class I PI3Ks, particularly PI3K-γ and PI3K-δ isoforms, are highly expressed in leukocytes and have been associated with adaptive and innate immunity; thus, these PI3Ks are believed to be important mediators in inflammatory disorders and hematologic malignancies (reviewed in Harris, S J et al. (2009) Curr Opin Investig Drugs 10(11):1151-62); Rommel C. et al. (2007) Nat Rev Immunol 7(3):191-201; Durand C A et al. (2009) J Immunol. 183(9):5673-84; Dil N, Marshall A J. (2009) MolImmunol. 46(10):1970-8; Al-Alwan M M et al. (2007) J Immunol. 178(4):2328-35; Zhang T T, et al. (2008) JAllergy Clin Immunol. 2008; 122(4):811-819.e2; Srinivasan L, et al. (2009) Cell 139(3):573-86)

PI3K-γ is a Class 1B PI3K that associates with the p101 and p84 (p87PIKAP) adaptor proteins, and canonically signals through GPCRs. Non-canonical activation through tyrosine kinase receptors and RAS can occur. Activated PI3K-γ leads to production of PIP3, which serves as a docking site for downstream effector proteins including AKT and BTK, bringing these enzymes to the cell membrane where they may be activated. A scaffolding role for PI3K-γ has been proposed and may contribute to the activation of the RAS/MEK/ERK pathway. The interaction with the RAS pathway explains activities attributed to kinase dead PI3K-γ in cells or in animals. PI3K-γ is essential for function of a variety of immune cells and pathways. Chemokine responses (including IL-8, fMLP, and C5a), leading to neutrophil, basophil or monocyte cell migration, is dependent on PI3K-γ (HIRSCH et al., “Central Role for G Protein-Coupled Phosphoinositide 3-Kinase γ in Inflammation,” Science 287:1049-1053 (2000); SASAKI et al., “Function of PI3Kγ in Thymocyte Development, T Cell Activation, and Neutrophil Migration,” Science 287:1040-1046 (2000); L I et al., “Roles of PLC-β2 and -β3 and PI3Kγ in Chemoattractant-Mediated Signal Transduction,” Science 287:1046-1049 (2000)). The requirement for PI3K-γ-dependent neutrophil migration is demonstrated by failure of arthritis development in the K/BXN serum transfer arthritis model in PI3K-γ knockout mice (Randis et al., Eur. J. Immunol., 2008, 38(5), 1215-24). Similarly, the mice fail to develop cellular inflammation and airway hyper-responsiveness in the ovalbumin induced asthma model (Takeda et al., J. Allergy Clin. Immunol., 2009; 123, 805-12). PI3K-γ deficient mice also have defects in T-helper cell function. T-cell cytokine production and proliferation in response to activation is reduced, and T helper dependent viral clearance is defective (Sasaki et al., Science, 2000, 287, 1040-46). T cell dependent inflammatory disease models including EAE also do not develop in PI3K-γ deficient mice, and both the T-cell activation defect and cellular migration defects may contribute to efficacy in this model (Comerfold, PLOS One, 2012, 7, e45095). The imiquimod psoriasis model has also been used to demonstrate the importance of PI3K-γ in the inflammatory response. Using PI3K-γ deficient mice in this model, the accumulation of γδ T cells in the skin is blocked, as well as dendritic cell maturation and migration (ROLLER et al., “Blockade of Phosphatidylinositol 3-Kinase (PI3K)δ or PI3Kγ Reduces IL-17 and Ameliorates Imiquimod-Induced Psoriasis-like Dermatitis,” J. Immunol. 189:4612-4620 (2012)). The role of PI3K-γ in cellular trafficking can also be demonstrated in oncology models where tumor inflammation is important for growth and metastasis of cancers. In the Lewis Lung Carcinoma model, monocyte activation, migration, and differentiation in tumors are defective. This defect results in a reduction in tumor growth and extended survival in PI3K-γ deficient mice (Schmid et al., Cancer Cell, 2011, 19, 715-27) or upon treatment with inhibitors that target PI3K-γ. In pancreatic cancer, PI3K-γ can be inappropriately expressed, and in this solid tumor cancer or others where PI3K-γ plays a functional role, inhibition of PI3K-γ can be beneficial.

For instance, while not wishing to be bound by theory, PI3K-γ is expressed in Gr1+CD11b+ myeloid cells, and directly promotes myeloid cell invasion and consequently, immunosuppression of pancreatic ductal carcinomas. Hardamon et. al., Proceedings: AACR 103rd Annual Meeting 2012, Cancer Research: Apr. 15, 2012; Volume 72, Issue 8, Supplement 1. Inhibition of PI3K-γ also shows promise for the treatment of hematologic malignancies. In a T-ALL model employing a T cell directed knockout of pten, PI3K-δ and PI3K-γ are both essential for the appropriate development of disease, as shown with genetic deletion of both genes (Subramaniam et al. Cancer Cell 21, 459-472, 2012). In addition, in this T-ALL model, treatment with a small molecule inhibitor of both kinases leads to extended survival of these mice. In CLL, chemokine networks support a pseudo-follicular microenvironment that includes Nurse like cells, stromal cells and T-helper cells. The roles of PI3K-γ in the normal chemokine signaling and T cell biology suggest the value of inhibiting this target in CLL (BURGER, “Inhibiting B-Cell Receptor Signaling Pathways in Chronic Lymphocytic Leukemia,” Curr. Mematol. Malig. Rep. 7:26-33 (2012)). Accordingly, PI3K-γ inhibitors are therapeutically interesting for diseases of the immune system where cell trafficking and T cell or myeloid cell function is important. In oncology, solid tumors that are dependent on tumor inflammation, or tumors with high levels of PI3K-γ expression, can be targeted. For hematological cancers, a special role for PI3K-γ and PI3K-δ isoforms in TALL and potentially in CLL suggests targeting these PI3Ks in these diseases.

Without being limited by a particular theory, PI3K-γ has been shown to play roles in cancer (e.g., Ruckle et al., Nature Rev., Drug Discovery, 2006, 5, 903-18; Schmid et al., “Myeloid cells in tumor inflammation,” Vascular Cell, 2012, doi:10.1186/2045-824×-4-14). For example, PI3K-γ functions in multiple signaling pathways involved in leukocyte activation and migration. In cancers, pharmacological or genetic blockade of p110γ suppresses inflammation, growth, and metastasis of implanted and spontaneous tumors, suggesting that PI3K-γ can be an important therapeutic target in oncology (Schmid et al., Cancer Cell, 2011, 19, 715-27). For example, it is shown that PI3K-γ has a tumor-specific high accumulation in pancreatic ductal adenocarcinoma (PDAC) in human, signifying a role of PI3K-γ in pancreatic cancer (Edling et al., Human Cancer Biology, 2010, 16(2), 4928-37).

In one embodiment, the subject has or is at risk of having a PI3K-gamma mediated disorder selected from cancer. In one embodiment, the cancer is a solid tumor. In one embodiment, the cancer is selected from one or more of: a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an esophageal cancer, a hepatocellular cancer, a gastric cancer, a gastrointestinal stromal tumor (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumor, diffuse type giant cell tumor, and glioblastoma.

Provided herein are methods of treating or preventing a (PD-L1 negative) cancer in a subject using the PI3K gamma inhibitor or a compound as described herein (e.g., Compound 1). In certain embodiments, the cancer is, or is identified as being, a solid tumor (e.g., lung cancer, melanoma, breast cancer, sarcoma, hepatocellular cancer, head and neck cancer, cervical or vulvar cancer, esophageal cancer, gastric cancer, adrenal cancer, colon cancer, or glioblastoma) or a hematologic cancer (e.g., a chronic lymphocytic leukemia (CLL)), e.g., as described herein. In one embodiment, the cancer is melanoma, bladder cancer, head and neck cancer, lung cancer (e.g., non-small cell lung cancer), renal cell carcinoma, ovarian cancer, breast cancer (e.g., triple-negative breast cancer), colon cancer, glioblastoma, gallbladder carcinoma, adrenocortical carcinoma, mesothelioma, endometrial cancer, or urothelial carcinoma.

In other embodiments, a method of reducing CXCL12-induced CD3+ T cell migration, or CXCL12-induced differentiated macrophage migration into a tumor microenvironment, in a subject is provided. The method includes administering to the subject a PI3K gamma inhibitor or a compound as described herein (e.g., Compound 1), in an amount sufficient to reduce or inhibit the CXCL12-induced CD3+ T cell migration, or CXCL12-induced differentiated macrophage migration into a tumor microenvironment in the subject.

In some embodiments of the methods or uses provided herein, the subject has, or is identified as having, a reduction in p-AKT levels after administration of the PI3K gamma inhibitor or a compound as described herein.

In some embodiments, a method of reducing one or more activities of a pro-tumor immune cell in a subject having a cancer is provided. The method includes administering to the subject a PI3K gamma inhibitor or a compound as described herein (e.g., Compound 1), in an amount sufficient to reduce or inhibit the one or more activities of the pro-tumor immune cell.

In some embodiments, the pro-tumor immune cell is a T-cell, an M2 macrophage, a stromal cell, a dendritic cell, an endothelial cell, or a myeloid cell. In one embodiment, the myeloid cell is a tumor associated suppressive myeloid cell. In one embodiment, the tumor associated suppressive myeloid cell is a tumor associated macrophage (TAM), a myeloid derived suppressor cell (MDSC), a monocytic immature myeloid cell (iMc), or a granulocytic iMc/neutrophil.

In certain embodiments, the subject has, or is identified as having, a decrease in numbers of pro-tumor immune cells in a tumor microenvironment, compared to a reference value, after administration of the PI3K gamma inhibitor or a compound as described herein.

In certain embodiments, the amount of the administered is sufficient to produce a decrease in numbers of pro-tumor immune cells in a tumor microenvironment, compared to a reference value, after administration of the PI3K gamma inhibitor or a compound as described herein.

In certain embodiments, the subject has, or is identified as having, increased activity of anti-tumor immune cells, compared to a reference value, after administration of the PI3K gamma inhibitor or a compound as described herein.

In certain embodiments, the amount of the PI3K gamma inhibitor or a compound as described herein is sufficient to produce increased activity of anti-tumor immune cells, compared to a reference value, after administration of the PI3K gamma inhibitor or the compound as described herein.

In certain embodiments, the subject has, or is identified as having, increased infiltration of anti-tumor immune cells into a tumor microenvironment, compared to a reference value, after administration of the PI3K gamma inhibitor or a compound as described herein.

In certain embodiments, the amount of PI3K-gamma inhibitor is sufficient to produce increased infiltration of anti-tumor immune cells into a tumor microenvironment, compared to a reference value, after administration of the PI3K gamma inhibitor or a compound as described herein.

In certain embodiments, the subject has, or is identified as having, an increase in number of anti-tumor immune cells in a tumor microenvironment, compared to a reference value, after administration of the PI3K gamma inhibitor or a compound as described herein.

In certain embodiments, the amount of PI3K-gamma inhibitor is sufficient to produce an increase in number of anti-tumor immune cells in a tumor microenvironment, compared to a reference value, after administration of the PI3K gamma inhibitor or a compound as described herein.

In certain embodiments, the cancer is a CLL. In some embodiments, the tumor microenvironment is a CLL proliferation center.

In certain embodiments, the subject has, or is identified as having, reduced tumor volume, compared to a reference value, after administration of the PI3K gamma inhibitor or a compound as described herein.

In certain embodiments, the amount of the PI3K gamma inhibitor or a compound as described herein is sufficient to produce reduced tumor volume, compared to a reference value, after administration of the PI3K gamma inhibitor or the compound as described herein.

In certain embodiments, the amount of the PI3K gamma inhibitor or a compound as described herein is sufficient to produce a reduction of at least 10%, 20%, 30%, 50%, 60%, or 60% in tumor volume, compared to a reference value, after administration of the PI3K gamma inhibitor or the compound as described herein.

In certain embodiments, the subject has, or is identified as having, an increased level of apoptosis in the cancer cells, compared to a reference value, after administration of the PI3K gamma inhibitor or a compound as described herein.

In certain embodiments, the amount of PI3K gamma inhibitor is sufficient to produce an increased level of apoptosis in the cancer cells, compared to a reference value, after administration of the PI3K gamma inhibitor or a compound as described herein.

In certain embodiments, the subject has, or is identified as having, a 10%, 20%, 30%, 40%, or 50% increase in apoptosis in the cancer cells, compared to a reference value, after administration of the PI3K gamma inhibitor or a compound as described herein.

In certain embodiments, the amount of the PI3K gamma inhibitor or a compound as described herein is sufficient to produce a 10%, 20%, 30%, 40%, or 50% increase in apoptosis in the cancer cells, compared to a reference value, after administration of the PI3K gamma inhibitor or the compound as described herein.

In certain embodiments, the anti-tumor immune cell is an M1 macrophage.

In certain embodiments, the one activity is chosen from one or more of migration of the cell, or signaling to an anti-tumor immune cell.

In certain embodiments, the subject has, or is determined to have reduced levels of p-AKT in the pro-tumor immune cell, compared to a reference value, after administration of the PI3K gamma inhibitor or the compound.

In certain embodiments, the amount is sufficient to reduce p-AKT in the pro-tumor immune cell, compared to a reference value, after administration of the PI3K gamma inhibitor or the compound.

In certain embodiments, the subject has, or is determined to have a reduction of p-AKT levels by about 10%, 20%, 30%, 40%, 50%, or 60%, compared to a reference value, after administration of the PI3K gamma inhibitor or the compound.

In certain embodiments, the subject has, or is determined to have a reduction of p-AKT levels by about 10%, 20%, 30%, 40%, 50%, or 60%, compared to a reference value, after administration of the PI3K gamma inhibitor or the compound.

In certain embodiments, the subject has, or is determined to have (PD-L1 negative) melanoma, lung cancer, head and neck cancer, renal cell carcinoma, gallbladder carcinoma, breast cancer, colon cancer, glioblastoma, adrenocortical carcinoma, mesothelioma, colorectal cancer, ovarian cancer, endometrial cancer, or urothelial carcinoma. In certain embodiments, the breast cancer is triple negative breast cancer.

In some embodiments, a method of reducing the level of M2 macrophages in a tumor microenvironment in a subject having a cancer is provided. The method includes administering to the subject a PI3K gamma inhibitor or a compound as described herein (e.g., Compound 1), in an amount sufficient to reduce the level of M2 macrophages in a tumor microenvironment.

In certain embodiments, reducing the level of M2 macrophages comprises reducing or inhibiting the differentiation of a tumor associated myeloid cell into an M2 macrophage. Differentiation into an M2 macrophage can be measured by decreased ARG1 levels compared to a reference value, after administration of the compound.

In certain embodiments, the ARG1 level is reduced by at least 10%, 20%, 30%, 40%, 50%, 60%, 70%, 80%, or 90% compared to a reference value, after administration of the compound.

In certain embodiments, differentiation into an M2 macrophage is measured by decreased VEGF levels compared to a reference value, after administration of the compound.

In certain embodiments, the VEGF level is reduced by at least 10%, 20%, 30%, 40%, 50%, 60%, 70%, 80%, or 90% compared to a reference value, after administration of the compound.

In certain embodiments, the subject has, or is determined to have, a normal level of differentiation of myeloid cells into M1 macrophages.

In certain embodiments, the amount is such that the compound does not reduce differentiation of myeloid cells into M1 macrophages.

In certain embodiments, the subject has, or is determined to have, increased anti-tumor immune attack by effector T cells, reduced vascularization of a tumor, reduced ECM breakdown, decreased tumor growth, or any combination thereof, compared to a reference value, after administration of the compound.

Class I PI3Ks, particularly PI3K-δ and PI3K-γ isoforms, are also associated with cancers (reviewed, e.g., in Vogt, P K et al. (2010) Curr Top Microbiol Immunol. 347:79-104; Fresno Vara, J A et al. (2004) Cancer Treat Rev. 30(2):193-204; Zhao, L and Vogt, PK. (2008) Oncogene 27(41):5486-96). Inhibitors of PI3K, e.g., PI3K-δ and/or PI3K-γ, have been shown to have anti-cancer activity (e.g., Courtney, K D et al. (2010) J Clin Oncol. 28(6):1075-1083); Markman, B et al. (2010) Ann Oncol. 21(4):683-91; Kong, D and Yamori, T (2009) Curr Med Chem. 16(22):2839-54; Jimeno, A et al. (2009) J Clin Oncol. 27:156s (suppl; abstr 3542); Flinn, I W et al. (2009) J Clin Oncol. 27:156s (suppl; abstr 3543); Shapiro, G et al. (2009) J Clin Oncol. 27:146s (suppl; abstr 3500); Wagner, A J et al. (2009) J Clin Oncol. 27:146s (suppl; abstr 3501); Vogt, P K et al. (2006) Virology 344(1):131-8; Ward, S et al. (2003) Chem Biol. 10(3):207-13; WO 2011/041399; US 2010/0029693; US 2010/0305096; US 2010/0305084; each incorporated herein by reference).

In one embodiment, described herein is a method of treating (PD-L1 negative) cancer. In one embodiment, provided herein is a method of treating a hematological caner comprising administering a pharmaceutically effective amount of a compound provided herein to a subject in need thereof. In one embodiment, provided herein is a method of treating a (PD-L1 negative) solid tumor comprising administering a pharmaceutically effective amount of a compound provided herein to a subject in need thereof. Types of cancer that can be treated with an inhibitor of PI3K (particularly, PI3K-δ and/or PI3K-γ) include, e.g., leukemia, chronic lymphocytic leukemia, acute myeloid leukemia, chronic myeloid leukemia (e.g., Salmena, L et al. (2008) Cell 133:403-414; Chapuis, N et al. (2010) Clin Cancer Res. 16(22):5424-35; Khwaja, A (2010) Curr Top Microbiol Immunol. 347:169-88); lymphoma, e.g., non-Hodgkin's lymphoma (e.g., Salmena, L et al. (2008) Cell 133:403-414); lung cancer, e.g., non-small cell lung cancer, small cell lung cancer (e.g., Herrera, V A et al. (2011) Anticancer Res. 31(3):849-54); melanoma (e.g., Haluska, F et al. (2007) Semin Oncol. 34(6):546-54); prostate cancer (e.g., Sarker, D et al. (2009) Clin Cancer Res. 15(15):4799-805); glioblastoma (e.g., Chen, J S et al. (2008) Mol Cancer Ther. 7:841-850); endometrial cancer (e.g., Bansal, N et al. (2009) Cancer Control. 16(1):8-13); pancreatic cancer (e.g., Furukawa, T (2008) J Gastroenterol. 43(12):905-11); renal cell carcinoma (e.g., Porta, C and Figlin, R A (2009) J Urol. 182(6):2569-77); colorectal cancer (e.g., Saif, M W and Chu, E (2010) Cancer J 16(3):196-201); breast cancer (e.g., Torbett, N E et al. (2008) Biochem J. 415:97-100); thyroid cancer (e.g., Brzezianska, E and Pastuszak-Lewandoska, D (2011) Front Biosci. 16:422-39); and ovarian cancer (e.g., Mazzoletti, M and Broggini, M (2010) Curr Med Chem. 17(36):4433-47).

Numerous publications support a role of PI3K-δ and PI3K-γ in treating hematological cancers. PI3K-δ and PI3K-γ are highly expressed in the heme compartment, and solid tumors, including prostate, breast and glioblastomas (Chen J. S. et al. (2008) Mol Cancer Ther. 7(4):841-50; Ikeda H. et al. (2010) Blood 116(9):1460-8).

In hematological cancers including acute myeloid leukemia (AML), multiple myeloma (MM), and chronic lymphocytic leukemia (CLL), overexpression and constitutive activation of PI3K-δ supports the model that PI3K-δ inhibition would be therapeutic Billottet C, et al. (2006) Oncogene 25(50):6648-59; Billottet C, et al. (2009) Cancer Res. 69(3):1027-36; Meadows, SA, 52^{nd} Annual ASH Meeting and Exposition; 2010 Dec. 4-7; Orlando, FL; Ikeda H, et al. (2010) Blood 116(9):1460-8; Herman S E et al. (2010) Blood 116(12):2078-88; Herman S E et al. (2011). Blood 117(16):4323-7.

Another mechanism for PI3K inhibitors to have an effect in solid tumors involves the tumor cells' interaction with their micro-environment. PI3K-δ, PI3K-γ, and PI3K-β are expressed in the immune cells that infiltrate tumors, including tumor infiltrating lymphocytes, macrophages, and neutrophils. PI3K-δ inhibitors can modify the function of these tumor-associated immune cells and how they respond to signals from the stroma, the tumor, and each other, and in this way affect tumor cells and metastasis (Hoellenriegel, J, et al. 5^{2nd} Annual ASH Meeting and Exposition; 2010 Dec. 4-7; Orlando, FL).

The role of PI3K-γ pathway in promoting myeloid cell trafficking to tumors and the role of blockade of p100γ in suppression of tumor inflammation and growth in breast cancer, pancreatic cancer, and lung cancer are reported, for example, in Schmid et al. (2011) Cancer Cell 19, 715-727, the entirety of which is incorporated herein by reference. In one embodiment, provided herein is a method of treating or preventing pancreatic cancer with a PI3K inhibitor.

While not wishing to be bound by theory, it is believed that tumor growth is influenced by two classes of immune cells in the tumor microenvironment: effector cells which include cytotoxic cells and M1 macrophages, and which have anti-tumor activity, and suppressor cells, which include M2 macrophages, MDSC (myeloid derived suppressor cell), Tregs (regulatory T cell), and regulatory dendritic cells, which have pro-tumor activity because they inhibit the effector cells. An abundance of suppressor cells can lead to tumor immune tolerance, and enhancement of tumor growth.

Certain of these cell types are briefly described. M1 denotes a pro-inflammatory (anti-tumor) phenotype of a MDSC or TAM. M2 denotes an anti-inflammatory (pro-tumor) phenotype of a MDSC or TAM.

PI3K-γ is not expressed in at least some cancer cell types. Schmid et al., 2011, Cancer Cell 19. Accordingly, in some embodiments, the PI3K-γ inhibitor reduces cancer cell growth without having a substantial direct effect on the cancer cell itself. For instance, in some embodiments, the PI3K-γ inhibitor inhibits cancer cell growth through changes in the tumor microenvironment, e.g., the immune cells in close proximity to the cancer cells.

Evidence provided herein, combined with evidence in the literature, support the idea that a PI3K-γ inhibitor can reduce tumor associated myeloid cells. For instance, in PI3K-γ-deficient mice, tumor-associated myeloid cells are reduced. Schmid et al., 2011, Cancer Cell 19. Together, these data indicate that a large class of PI3K-γ inhibitors should reduce tumor associated myeloid cells, thereby increasing the immune response against cancer cells, and treating the cancer. While not wishing to be bound by theory, a PI3K-γ may operate through the following mechanism. PI3K-γ signaling may tilt the balance of immune cells towards pro-tumor M2 cells and away from anti-tumor M1 cells, by inducing expression of immunosuppressive, wound healing genes such as Arginase1, TGFbeta1, PDGFBB, MMP9, and MMP13, and suppressing pro-inflammatory factors such as IL12, iNos, and interferon gamma. Blocking PI3K-γ signaling with an inhibitor tilts the balance towards anti-tumor M1 cells by stimulating a T cell activating gene expression program. Kaneda et al. PI3-kinase gamma controls the macrophage M1-M2 switch, thereby promoting tumor immunosuppression and progression. [abstract]. In: Proceedings of the 105th Annual Meeting of the American Association for Cancer Research; 2014 Apr. 5-9; San Diego, CA. Philadelphia (PA): AACR; Cancer Res 2014; 74(19 Suppl):Abstract nr 3650. doi:10.1158/1538-7445.AM2014-3650.

In some embodiments, a PI3K-γ inhibitor provided herein is administered to a patient in order to block a homeostatic down-regulation of T cell response. While not wishing to be bound by theory, this may allow the body to raise an effective immune response against the cancer cell. Exemplary agents of this type include immune checkpoint therapeutics, e.g., agents that act on CTLA-4, PD-1, or PD-L1, e.g., antibodies that bind to CTLA-4, PD-1, or PD-L1.

In some embodiments, a PI3K-γ inhibitor provided herein is administered to a patient in order to eliminate immunosuppressive cells in the tumor microenvironment. The immunosuppressive cell may be, e.g., a T regulatory cell (e.g., a cell that secretes mediators that induce CD8+ cytotoxic T cell death); a Tumor-associated macrophage (TAM; e.g., an M2 (pro-tumor) TAMS that blocks T cell activity and promotes angiogenesis); or a myeloid-derived suppressor cell (MDSC; e.g., a cell that secretes mediators that inhibit T cell differentiation and proliferation).

In some embodiments, a compound provided herein is administered to a patient in order to reduce the migration or differentiation of a tumor associated myeloid cell. In some embodiments, the compound is a compound that shows single agent activity in a syngeneic model system. In some embodiments, the compound is administered in combination with a second therapeutic, as discussed herein. In some embodiments, the administration results in a reduction in the level of MDSCs in the tumor microenvironment; the level of M2 TAMS in the tumor microenvironment; the level of T-regulatory cells in the tumor microenvironment, or any combination thereof. In some embodiments, the administration results in an unchanged or increased level of T-effector cells in the tumor microenvironment. In embodiments, the administration results in an increase in an immune response to the tumor, e.g., an increase in the levels or tumor-attacking activity of cytotoxic T cells, M1 inflammatory TAMs, or a combination thereof.

In some embodiments, an MDSC has one or more of the following properties: suppressing anti-tumor immune attack; inducing vascularization of the tumor; inducing ECM breakdown, e.g., which may contribute to metastasis; and supporting tumor growth. Accordingly, in some embodiments, administration of a PI3K-γ inhibitor described herein inhibits one or more of these functions in an MDSC.

TAMs (tumor-associated macrophages) can also have one or more of the following properties: suppressing anti-tumor immune attack; inducing vascularization of the tumor; inducing ECM breakdown, e.g., which may contribute to metastasis; and supporting tumor growth. Accordingly, in some embodiments, administration of a PI3K-γ inhibitor as described herein inhibits one or more of these functions in a TAM.

In embodiments, a PI3K-γ inhibitor is administered to a patient who has received chemotherapy and/or radiation therapy. While not wishing to be bound by theory, in some embodiments, chemotherapy or radiation therapy results in a wound healing response that leads to repopulation of the cancer site, e.g., tumor, with TAMs and MDSCs. Administering the PI3K-γ inhibitor, in some embodiments, reduces the levels of TAMs and MDSCs in the microenvironment, decreasing their support for tumor cell growth and/or allowing the immune system to attack the cancer cells. See Claire E. Lewis, “Imaging immune cell infiltrating tumors in zebrafish”, AACR Annual Meeting (Apr. 5, 2014).

Without being bound by a particular theory, a rationale for the use of a PI3K inhibitor to treat or prevent cancer is that cells derived from tumors (e.g., from CT26 mouse tumors) can suppress anti-tumor immune cell function, including T-cell proliferation, and treatment with a compound provided herein can release the suppression. The tumor microenvironment can inhibit the activation and proliferation of immune effector cells due to the presence of suppressive myeloid cells (e.g., myeloid derived suppressor cells or MDSC and M2 macrophages). Compounds provided herein can affect the number and activity M2 macrophages in a tumor microenvironment, e.g., reduce or inhibit the level of M2, pro-tumor macrophages. The reduction or inhibition of M2 macrophages, which produce anti-inflammatory cytokines and other factors, would lead to increased anti-tumor immunity, including T cell proliferation. Therefore, a compound provided herein can treat or prevent cancer such as colon cancer, melanoma, bladder cancer, renal cancer, breast, lung cancer, glioblastoma, solid tumors, and a cancer of hematopoietic origin (e.g., lymphoma, DLBCL, CLL, Hodgkin disease, non-Hodgkin lymphomas). Further, it has also been shown in the examples provided herein that anti-PD-L1 can also release suppression of T cell proliferation by blocking the interaction between PD-1 on T cells and PD-L1 on tumor cells and regulatory cells. The cytotoxic T cells that are induced to proliferate and survive by both anti PD-L1 and compound 1 are hypothesized to slow tumor growth. Compounds provided herein can relieve immunosuppression which can lead to T cells proliferation and activation. Compounds provided herein can treat or prevent cancer by inducing T cell mediated immunity. In one embodiment, the compound provided herein can decrease tumor volume. In one embodiment, a combination of a PI3K inhibitor such as a compound provided herein and anti-PD-L1 would be effective in treating or preventing cancer by inducing T cell mediated tumor immunity. In some embodiments, the effect of a compound provided herein on T-cell function can be assessed by analyzing the pro-inflammatory cytokine levels in tumor tissues and serum, e.g., a MSD pro-inflammatory panel. In another embodiment, the pro-inflammatory cytokines are selected from IFN-γ, IL-1β, IL-10, IL-12 p70, IL-2, IL-4, IL-5, IL-6, KC/GRO, and TNF-α. In one embodiment, the effect of a compound provided herein on T cell function can be assessed by analyzing the IFN-γ level. For example, tumor tissues and serum treated with a compound provided herein, e.g., Compound 1, can be assessed by analyzing the IFN-γ level.

In certain embodiments, provided herein are methods of modulating tumor microenvironment of cancer cells in a subject, comprising administering to the subject a therapeutically effective amount of a compound provided herein (e.g., Compound 1), or a pharmaceutically acceptable form thereof.

As used herein and unless otherwise specified, “tumor microenvironment” refers to the cellular and extracellular environment where the tumors are located. This location can include surrounding blood vessels, immune cells, fibroblasts, secreted signaling molecules, and the extracelluar matrix. The tumor microenvironment includes non-neoplastic stromal and immune cells that provide growth and survival support to the neoplastic tumor.

As used herein and unless otherwise specified, “immunotherapy” refers to treatments that stimulate, enhance, or suppress the body's own immune system to fight a disease. Diseases that may be suitable for immunotherapy treatment include, but are not limited to, cancer, inflammatory diseases, and infectious diseases. Immunotherapy includes a variety of treatments that work in different ways. For example, some are intended to boost the immune system defenses in a general way; others help train the immune system to recognize and attack cancer cells specifically. Cancer immunotherapies include, but are not limited to, cell-based therapies (also known as cancer vaccines), antibody therapies, and cytokine therapies (e.g., interleukin-2 and interferon-α).

Many cancers are known to be susceptible to the treatment of one or more immunotherapies, including treatment targeting the effector cells in the tumor microenvironment (e.g., immune checkpoint therapy such as PD-1/PD-L1 inhibitors and CTLA-4 inhibitors), treatment targeting suppressor cells in the tumor microenvironment (e.g., CSF-1R inhibitors (affecting MDSC and TAM) and IDO/TDO inhibitors). Without being limited by a particular theory, a compound provided herein (e.g., Compound 1) may affect MDSC, TAM, and other components in the tumor microenvironment. The role of TAM in tumor microenvironment is described, e.g., in Lewis and Pollard, Cancer Res. 2006; 66: (2). Jan. 15, 2006.

In one embodiment, the number of one or more pro-tumor immune cells in the tumor microenvironment is reduced, or the activity of one or more pro-tumor immune cells in the tumor microenvironment is reduced or inhibited, after administration of the compound. In some embodiments, the pro-tumor immune cell is a T-cell, an M2 macrophage, a stromal cell, a dendritic cell, an endothelial cell, or a myeloid cell. In one embodiment, the myeloid cell is a tumor associated suppressive myeloid cell. In one embodiment, the tumor associated suppressive myeloid cell is identified by (i) CD45+, CD11b+, Ly6C+ and Ly6G+, (ii) CD45+, CD11b+, Ly6C− and Ly6G−, (iii) CD45+, CD11b+, Ly6C− and Ly6G+, or (iv) CD45+, CD11b+, Ly6C+ and Ly6G−. In one embodiment, the tumor associated suppressive myeloid cell is a tumor associated macrophage (TAM), a myeloid derived suppressor cell (MDSC), a monocytic immature myeloid cell (iMc), or a granulocytic iMc/neutrophil. In one embodiment, the TAM is identified by CD45+, CD11b+, Ly6C−, and Ly6G−. In one embodiment, the myeloid derived suppressor cell (MDSC) is identified by CD45+, CD11b+, Ly6C− and Ly6G+. In one embodiment, the monocytic immature myeloid cell (iMc) is identified by CD45+, CD11b+, Ly6C+ and Ly6G−. In one embodiment, the granulocytic iMc/neutrophil is identified by CD45+, CD11b+, Ly6C+ and Ly6G+. See e.g., Coussens L M. et al., Cancer Discov. 2011 June; 1(1):54-67.

In one embodiment, the activation of M2 macrophage in the tumor microenvironment is reduced or inhibited after administration of the compound. In one embodiment, the p-AKT level in the M2 macrophage is reduced after administration of the compound. In one embodiment, the number of M2 macrophage cells in the tumor microenvironment is reduced after administration of the compound. In one embodiment, the migration of M2 macrophage cells into the tumor microenvironment is reduced or inhibited after administration of the compound. In one embodiment, the differentiation of myeloid cells into M2 macrophage cells in the tumor microenvironment is reduced or inhibited after administration of the compound. In one embodiment, the differentiation into M2 macrophage cells is measured by Arginase-1 (ARG1) level or VEGF level, and the ARG1 level or VEGF level is reduced by at least 10%, 20%, 30%, 40%, 50%, 60%, 70%, 80%, or 90% compared to a reference value.

In one embodiment, the number of myeloid-derived suppressor cells in the tumor microenvironment is reduced after administration of the compound. In one embodiment, the differentiation of bone marrow cells into myeloid-derived suppressor cells is reduced or inhibited after administration of the compound. In one embodiment, the differentiation into myeloid-derived suppressor cells is measured by Arginase-1 (ARG1) level, VEGF level, or iNOS level, and the ARG1 level, VEGF level, or iNOS level is reduced by at least 10%, 20%, 30%, 40%, 50%, 60%, 70%, 80%, or 90% compared to a reference value.

In one embodiment, the production of proangiogeneic factor is reduced or inhibited after administration of the compound. In one embodiment, the proangiogeneic factor is reduced or inhibited by reduction or inhibition of macrophage or MDSC differentiation. In one embodiment, the proangiogeneic factor is VEGF.

In one embodiment, the effect of the compounds provided herein (e.g., Compound 1) on MDSC (e.g., human MDSC) function is measured by expression of iNOS and arginase and production of ROS and IL-10, measured by the suppression function of the MDSC (e.g., in co-culture assays with CD8+), measured by activation of pAKT in response to a stimulant (e.g., CXCL12, IL-1b, TNF-α, or CSF1), or measured by transwell chemotaxis assays (T cells and MDSC).

In one embodiment, the effect of the compounds provided herein (e.g., Compound 1) on MDSC (e.g., murine MDSC) function and macrophage M2-polarization is measured by isolating myeloid cells from bone marrow, polarizing with IFNg or IL-4 and then testing for secretion of TNF-α, IL-12, ROS production in M1 and IL-10, IL-1b, or VEGF, or measured by methods provided herein or elsewhere.

In one embodiment, the effect of the compounds provided herein (e.g., Compound 1) on myeloid and CD8+ is measured by in vivo models (e.g., MC38 and 4T1). In one embodiment, the effect is measured by TGI, MDSC and macrophage infiltrate, CD8+, and IFN-gamma production in CD8+.

In one embodiment, the effect of the compounds provided herein (e.g., Compound 1) on myeloid and CD8+ is measured by QT-PCR or intracellular FACS of myeloid infiltrate. In one embodiment, the effect is measured by expression of functional markers (e.g., iNOS, arginase, or IL-10).

In one embodiment, the number of one or more anti-tumor immune cells in the tumor microenvironment is increased, or the activity of one or more anti-tumor immune cells in the tumor microenvironment is increased, after administration of the compound.

In one embodiment, the cancer susceptible to the treatment of one or more immunotherapies is a hematological cancer. In one embodiment, the hematological cancer is chronic lymphocytic leukemia (CLL). In one embodiment, the tumor microenvironment is a CLL proliferation center. In one embodiment, the hematological cancer is lymphoma.

In one embodiment, the cancer susceptible to the treatment of one or more immunotherapies is a solid tumor. In one embodiment, the solid tumor is lung cancer, breast cancer, colon cancer, or glioblastoma. In one embodiment, the cancer is selected from one or more of: a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an esophageal cancer, a hepatocellular cancer, a gastric cancer, a gastrointestinal stromal tumor (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumor, diffuse type giant cell tumor, andglioblastoma. In one embodiment, the solid tumor is melanoma, bladder cancer, head and neck cancer, lung cancer (e.g., non-small cell lung cancer), renal cell carcinoma, ovarian cancer, breast cancer (e.g., triple-negative breast cancer), colon cancer, glioblastoma, gallbladder carcinoma, adrenocortical carcinoma, mesothelioma, colorectal cancer, endometrial cancer, or urothelial carcinoma.

In one embodiment, the solid tumor is melanoma. In one embodiment, the solid tumor is lung cancer. In one embodiment, the solid tumor is non-small cell lung cancer. In one embodiment, the solid tumor is renal cell carcinoma. Melanoma, lung cancer (e.g., non-small cell lung cancer), and renal cell carcinoma are known to be sensitive to immunotherapies. Data linking a poor prognosis to high TAM cell counts have been reported in breast, prostate, endometrial, bladder, kidney, esophageal, superficial, carcinoma, melanoma, and follicular lymphoma cancers. See e.g., Lewis and Pollard, Cancer Res. 2006; 66: (2). Jan. 15, 2006. One anti-PD-1 antibody drug, nivolumab (Opdivo—Bristol Myers Squibb), produced complete or partial responses in non-small-cell lung cancer, melanoma, and renal-cell cancer, in a clinical trial with a total of 296 patients.

In one embodiment, the solid tumor is head and neck cancer. Head and neck tumors tend to be highly immunogenic and have strong anti-PD-1/PD-L1 efficacy. In one embodiment, the solid tumor is bladder cancer. Bladder cancer also has strong anti-PD-1/PD-L1 efficacy. A high number of TAM cells has been associated with a poor prognosis and increased tumor angiogenesis in bladder cancer.

In one embodiment, the solid tumor is breast cancer. In one embodiment, the breast cancer is triple-negative breast cancer. A high number of TAM cells has been associated with a poor prognosis of breast cancer. See e.g., Lewis and Pollard, Cancer Res. 2006; 66: (2). Jan. 15, 2006. In one embodiment, the solid tumor is ovarian cancer. In one embodiment, the solid tumor is colon cancer. Breast cancer, ovarian cancer, and colon cancer are known to be sensitive to immunotherapies (e.g., bevacizumab and trastuzumab) and can also have anti-PD-1/PD-L1 efficacy.

In one embodiment, the solid tumor is glioblastoma. In one embodiment, the solid tumor is glioblastoma multiforme. It has been reported that PI3K-gamma expression is upregulated in brain microglia. Without being limited by a particular theory, PI3K-γ inhibitors provided herein (e.g., Compound 1) may have P-glycoprotein inhibitory activity and thus can cross the blood brain barrier.

In one embodiment, the anti-tumor immune attack by effector T cells is increased, vascularization of the tumor is reduced, extracellular matrix (ECM) breakdown is reduced, or tumor growth is decreased, compared to a reference value, after administration of the compound.

In one embodiment, the tumor volume of the cancer is reduced after administration of the compound. In one embodiment, the tumor volume of the cancer is reduced by at least 10%, 20%, 30%, 50%, 60%, or 60%, compared to a reference value.

In one embodiment, the level of apoptosis of the cancer cells is increased after administration of the compound. In one embodiment, the level of apoptosis of the cancer cells is increased by at least 10%, 20%, 30%, 40%, or 50%, compared to a reference value.

In some embodiments, provided herein is a method of treating a (PD-L1 negative) cancer of hematopoietic origin. In certain embodiments, the cancer of hematopoietic origin is lymphoma or leukemia. In some embodiments, the cancer of hematopoietic origin is selected from acute lymphocytic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM); peripheral T cell lymphomas (PTCL), adult T cell leukemia/lymphoma (ATLL), cutaneous T-cell lymphoma (CTCL), large granular lymphocytic leukemia (LGL), acute myelocytic leukemia (AML), Hodgkin lymphoma (HL), non-Hodgkin lymphoma (NHL), follicular lymphoma, diffuse large B-cell lymphoma (DLBCL), mantle cell lymphoma (MCL), mastocytosis (e.g., systemic mastocytosis), multiple myeloma (MM), myelodysplastic syndrome (MDS), myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelogenous leukemia (CML), chronic neutrophilic leukemia (CNL), and hypereosinophilic syndrome (HES)).

In some embodiments, provided herein is a method of treating a (PD-L1 negative) solid tumor. In some embodiments, the solid tumor is selected from ovarian cancer, colon cancer, fibrosarcoma, pancreatic cancer, lung cancer, breast cancer, lymphoma, melanoma, and glioblastoma. In some embodiment, the solid tumor is a CNS tumor. In one embodiment, the CNS tumor is glioblastoma. The ADME data provide herein indicate that a compound provide herein (e.g., Compound 1) may show good permeability cross blood-brain-barrier and can achieving efficacious concentration in a CNS tumor.

Exemplary solid tumors include, but are not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic andenocarcinoma, intraductal papillary mucinous neoplasm (IPMN)), prostate cancer (e.g., prostate adenocarcinoma), skin cancer (e.g., squamous cell carcinoma (SCC), keratoacanthoma (A), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma).

In some embodiments, the solid tumor is selected from ovarian cancer, colon cancer, fibrosarcoma, pancreatic cancer, lung cancer, breast cancer, lymphoma, melanoma, and glioblastoma.

Patients that can be treated with a compound provided herein, or a pharmaceutically acceptable form (e.g., pharmaceutically acceptable salts, hydrates, solvates, isomers, prodrugs, and isotopically labeled derivatives) thereof, or a pharmaceutical composition as provided herein, according to the methods as provided herein include, for example, but not limited to, patients that have been diagnosed as having breast cancer such as a ductal carcinoma, lobular carcinoma, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute lymphoblastic leukemia, chronic myelogenous leukemia, hairy cell leukemia, myelodysplasia, myeloproliferative disorders, NK cell leukemia (e.g., blastic plasmacytoid dendritic cell neoplasm), acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer; kidney cancer; thyroid cancer such as papillary, follicular, medullary and anaplastic; lymphoma such as diffuse large B-cell lymphoma, B-cell immunoblastic lymphoma, NK cell lymphoma (e.g., blastic plasmacytoid dendritic cell neoplasm), and Burkitt lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), oligodendroglioma, ependymoma, meningioma, lymphoma, schwannoma, and medulloblastoma; peripheral nervous system (PNS) cancers such as acoustic neuromas and malignant peripheral nerve sheath tumor (MPNST) including neurofibromas and schwannomas, malignant fibrocytoma, malignant fibrous histiocytoma, malignant meningioma, malignant mesothelioma, and malignant mixed Müllerian tumor; oral cavity and oropharyngeal cancers such as, hypopharyngeal cancer, laryngeal cancer, nasopharyngeal cancer, and oropharyngeal cancer; stomach cancers such as lymphomas, gastric stromal tumors, and carcinoid tumors; testicular cancers such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin lymphoma, non-Hodgkin lymphomas, carcinoids or carcinoid tumors; rectal cancer; and colon cancer.

Patients that can be treated with compounds provided herein, or pharmaceutically acceptable salt, ester, prodrug, solvate, hydrate or derivative of said compounds, according to the methods provided herein include, for example, patients that have been diagnosed as having conditions including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM); peripheral T cell lymphomas (PTCL), adult T cell leukemia/lymphoma (ATLL), cutaneous T-cell lymphoma (CTCL), large granular lymphocytic leukemia (LGL), acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelogenous leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenstrom's macroglobulinemia.

Without being limited by a particular theory, in one embodiment, the cancer or disease being treated or prevented, such as a blood disorder or hematologic malignancy, has a high expression level of one or more PI3K isoform(s) (e.g., PI3K-α, PI3K-β, PI3K-δ, or PI3K-γ, or a combination thereof). In one embodiment, the cancer or disease that can be treated or prevented by methods, compositions, or kits provided herein includes a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others. In one embodiment, the blood disorder or the hematologic malignancy includes, but is not limited to, acute lymphoblastic leukemia (ALL), T-cell ALL (T-ALL), B-cell ALL (B-ALL), acute myeloid leukemia (AML), chronic lymphocytic leukemia (CLL), chronic myelogenous leukemia (CML), blast phase CML, small lymphocytic lymphoma (SLL), CLL/SLL, transformed CLL, Richter syndrome Hodgkin lymphoma (HL), non-Hodgkin lymphoma (NHL), B-cell NHL, T-cell NHL, indolent NHL (iNHL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sézary syndrome, anaplastic large-cell lymphoma (ALCL), follicular lymphoma (FL), Waldenstrom macroglobulinemia (WM), lymphoplasmacytic lymphoma, Burkitt lymphoma, multiple myeloma (MM), amyloidosis, MPD, essential thrombocytosis (ET), myelofibrosis (MF), polycythemia vera (PV), chronic myelomonocytic leukemia (CMML), myelodysplastic syndrome (MDS), angioimmunoblastic lymphoma, high-risk MDS, and low-risk MDS. In one embodiment, the hematologic malignancy is relapsed. In one embodiment, the hematologic malignancy is refractory. In one embodiment, the cancer or disease is in a pediatric patient (including an infantile patient). In one embodiment, the cancer or disease is in an adult patient. Additional embodiments of a cancer or disease being treated or prevented by methods, compositions, or kits provided herein are described herein elsewhere.

In exemplary embodiments, the cancer or hematologic malignancy is CLL. In exemplary embodiments, the cancer or hematologic malignancy is CLL/SLL. In exemplary embodiments, the cancer or hematologic malignancy is transformed CLL or Richter syndrome. In exemplary embodiments, the cancer or hematologic malignancy is SLL. In one embodiment, without being limited by a particular theory, a compound provided herein (e.g., a PI3K-γ selective compound provided herein) inhibits the migration and/or activation of T-cells and myeloid cells (e.g., macrophages or polarized M2 macrophages), reducing survival and/or proliferative support provided by those cells to malignant CLL cells within the tumor microenvironment (TME). In one embodiment, without being limited by a particular theory, the migration of CD3+ T cells to the CLL-associated chemokine CXCL12 is blocked by a compound provided herein (e.g., a PI3K-γ selective compound provided herein). In another embodiment, without being limited by a particular theory, a compound provided herein (e.g., a PI3K-γ selective compound provided herein) block the myeloid cell mediated re-growth of a cancer following chemotherapy through its effects on inhibiting the post-chemotherapy migration of myeloid cells into a tumor.

In exemplary embodiments, the cancer or hematologic malignancy is iNHL. In exemplary embodiments, the cancer or hematologic malignancy is DLBCL. In exemplary embodiments, the cancer or hematologic malignancy is B-cell NHL (e.g., aggressive B-cell NHL). In exemplary embodiments, the cancer or hematologic malignancy is MCL. In exemplary embodiments, the cancer or hematologic malignancy is RS. In exemplary embodiments, the cancer or hematologic malignancy is AML. In exemplary embodiments, the cancer or hematologic malignancy is MM. In exemplary embodiments, the cancer or hematologic malignancy is ALL. In exemplary embodiments, the cancer or hematologic malignancy is T-ALL. In exemplary embodiments, the cancer or hematologic malignancy is B-ALL. In exemplary embodiments, the cancer or hematologic malignancy is TCL. In exemplary embodiments, the cancer or hematologic malignancy is ALCL. In exemplary embodiments, the cancer or hematologic malignancy is leukemia. In exemplary embodiments, the cancer or hematologic malignancy is lymphoma. In exemplary embodiments, the cancer or hematologic malignancy is T-cell lymphoma. In exemplary embodiments, the cancer or hematologic malignancy is MDS (e.g., low grade MDS). In exemplary embodiments, the cancer or hematologic malignancy is MPD. In exemplary embodiments, the cancer or hematologic malignancy is a mast cell disorder. In exemplary embodiments, the cancer or hematologic malignancy is Hodgkin lymphoma (HL). In exemplary embodiments, the cancer or hematologic malignancy is non-Hodgkin lymphoma. In exemplary embodiments, the cancer or hematologic malignancy is PTCL. In exemplary embodiments, the cancer or hematologic malignancy is CTCL (e.g., mycosis fungoides or Sézary syndrome). In exemplary embodiments, the cancer or hematologic malignancy is WM. In exemplary embodiments, the cancer or hematologic malignancy is CML. In exemplary embodiments, the cancer or hematologic malignancy is FL. In exemplary embodiments, the cancer or hematologic malignancy is transformed mycosis fungoides. In exemplary embodiments, the cancer or hematologic malignancy is Sézary syndrome. In exemplary embodiments, the cancer or hematologic malignancy is acute T-cell leukemia. In exemplary embodiments, the cancer or hematologic malignancy is acute B-cell leukemia. In exemplary embodiments, the cancer or hematologic malignancy is Burkitt lymphoma. In exemplary embodiments, the cancer or hematologic malignancy is myeloproliferative neoplasms. In exemplary embodiments, the cancer or hematologic malignancy is splenic marginal zone. In exemplary embodiments, the cancer or hematologic malignancy is nodal marginal zone. In exemplary embodiments, the cancer or hematologic malignancy is extranodal marginal zone.

In one embodiment, the cancer or hematologic malignancy is a B cell lymphoma. In a specific embodiment, provided herein is a method of treating or managing a B cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a B cell lymphoma comprising administering to a patient a therapeutically effective amount of a compound provided herein, or a pharmaceutically acceptable derivative (e.g., salt or solvate) thereof. In one embodiment, the B cell lymphoma is iNHL. In another embodiment, the B cell lymphoma is follicular lymphoma. In another embodiment, the B cell lymphoma is Waldenstrom macroglobulinemia (lymphoplasmacytic lymphoma). In another embodiment, the B cell lymphoma is marginal zone lymphoma (MZL). In another embodiment, the B cell lymphoma is MCL. In another embodiment, the B cell lymphoma is HL. In another embodiment, the B cell lymphoma is aNHL. In another embodiment, the B cell lymphoma is DLBCL. In another embodiment, the B cell lymphoma is Richters lymphoma.

In one embodiment, the cancer or hematologic malignancy is a T cell lymphoma. In a specific embodiment, provided herein is a method of treating or managing a T cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a T cell lymphoma comprising administering to a patient a therapeutically effective amount of a compound provided herein, or a pharmaceutically acceptable derivative (e.g., salt or solvate) thereof. In one embodiment, the T cell lymphoma is peripheral T cell lymphoma (PTCL). In another embodiment, the T cell lymphoma is cutaneous T cell lymphoma (CTCL).

In one embodiment, the cancer or hematologic malignancy is Sézary syndrome. In a specific embodiment, provided herein is a method of treating or managing Sézary syndrome comprising administering to a patient a therapeutically effective amount of a compound provided herein, or a pharmaceutically acceptable derivative (e.g., salt or solvate) thereof. Also provided herein is a method of treating or lessening one or more of the symptoms associated with Sézary syndrome comprising administering to a patient a therapeutically effective amount of a compound provided herein, or a pharmaceutically acceptable derivative (e.g., salt or solvate) thereof. The symptoms associated with Sézary syndrome include, but are not limited to, epidermotropism by neoplastic CD4+ lymphocytes, Pautrier's microabscesses, erythroderma, lymphadenopathy, atypical T cells in the peripheral blood, and hepatosplenomegaly.

In one embodiment, the cancer or hematologic malignancy is relapsed. In one embodiment, the cancer or hematologic malignancy is refractory. In certain embodiments, the cancer being treated or prevented is a specific sub-type of cancer described herein. In certain embodiments, the hematologic malignancy being treated or prevented is a specific sub-type of hematologic malignancy described herein. Certain classifications of type or sub-type of a cancer or hematologic malignancy provided herein is known in the art. Without being limited by a particular theory, it is believed that many of the cancers that become relapsed or refractory develop resistance to the particular prior therapy administered to treat the cancers. Thus, without being limited by a particular theory, a compound provided herein can provide a second line therapy by providing an alternative mechanism to treat cancers different from those mechanisms utilized by certain prior therapies. Accordingly, in one embodiment, provided herein is a method of treating or managing cancer or hematologic malignancy comprising administering to a patient a therapeutically effective amount of a compound provided herein, or a pharmaceutically acceptable derivative (e.g., salt or solvate) thereof, wherein the cancer or hematologic malignancy is relapsed after, or refractory to, a prior therapy.

In exemplary embodiments, the cancer or hematologic malignancy is refractory iNHL. In exemplary embodiments, the cancer or hematologic malignancy is refractory CLL. In exemplary embodiments, the cancer or hematologic malignancy is refractory SLL. In exemplary embodiments, the cancer or hematologic malignancy is refractory to rituximab therapy. In exemplary embodiments, the cancer or hematologic malignancy is refractory to chemotherapy. In exemplary embodiments, the cancer or hematologic malignancy is refractory to radioimmunotherapy (RIT). In exemplary embodiments, the cancer or hematologic malignancy is iNHL, FL, splenic marginal zone, nodal marginal zone, extranodal marginal zone, or SLL, the cancer or hematologic malignancy is refractory to rituximab therapy, chemotherapy, and/or RIT.

In another exemplary embodiment, the cancer or hematologic malignancy is lymphoma, and the cancer is relapsed after, or refractory to, the treatment by a BTK inhibitor such as, but not limited to, ibrutinib or ONO-4059. In another exemplary embodiment, the cancer or hematologic malignancy is CLL, and the cancer is relapsed after, or refractory to, the treatment by a BTK inhibitor such as, but not limited to, ibrutinib and AVL-292.

In one embodiment, the solid tumor is selected from one or more of: a cancer of the pulmonary system, a brain cancer, a cancer of the gastrointestinal tract, a skin cancer, a genitourinary cancer, a pancreatic cancer, a lung cancer, a medullobastoma, a basal cell carcinoma, a glioma, a breast cancer, a prostate cancer, a testicular cancer, an esophageal cancer, a hepatocellular cancer, a gastric cancer, a gastrointestinal stromal tumor (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumor, diffuse type giant cell tumor, and glioblastoma.

In some embodiments, the subject is naive to immunotherapy treatment. In some embodiments, the subject is or has been responsive to an immunotherapy treatment. In some embodiments, the subject is relapsed or refractory to an immunotherapy treatment. In one embodiment, the immunotherapy treatment is a treatment with a PD-1 or PD-L1 inhibitor.

In some embodiments, the subject is naive to radiation therapy treatment. In some embodiments, the subject is naive to chemotherapy treatment.

In some embodiments, the subject has been pre-treated or previously treated with one or more immunotherapy treatments. In one embodiment, the subject is responsive to the pre-treatment or previous treatment with the immunotherapy. In one embodiment, the immunotherapy treatment is a checkpoint treatment such as a PD-1 or PD-L1 inhibitor. In one embodiment, the subject is a smoker. It has been reported that smoker patients may respond better to immunotherapy (e.g., a PD-L1 inhibitor MPDL3280A) than non-smoker patients in a phase I clinical study for patients with melanoma or cancers of the lung, kidney, colon, GI tract, or head and neck cancers.

In one embodiment, the cancer is melanoma, and the subject has been pre-treated or previously treated with one or more immunotherapy treatments. In one embodiment, the subject has been pre-treated or previously treated with two or more immunotherapy treatments.

In one embodiment, the cancer is head and neck cancer, lung cancer (e.g., non-small cell lung cancer), renal cell carcinoma, or bladder cancer, and the subject has been pre-treated or previously treated with one immunotherapy treatment.

In one embodiment, the cancer is breast cancer (e.g., triple-negative breast cancer), ovarian cancer, glioblastoma, or colon cancer, and the subject is naive to immunotherapy treatment.

In one embodiment, provided herein is a method of treating, preventing, or managing (PD-L1 negative) melanoma in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is ipilimumab (Yervoy), interleukin-2, vemurafenib, dabrafenib, or trametinib.

In one embodiment, provided herein is a method of treating, preventing, or managing (PD-L1 negative) lung cancer (e.g., non-small cell lung cancer) in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is bevacizumab, erlotinib, gefitinib, afatinib, or denosumab.

In one embodiment, provided herein is a method of treating, preventing, or managing (PD-L1 negative) renal cell carcinoma in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is bevacizumab, interleukin-2, axitinib, carfilzomib, everolimus, interferon-α, lenalidomide, pazopanib, sirolimus (rapamycin), sorafenib, sunitinib, temsirolimus, thalidomide, or tivozanib.

In one embodiment, provided herein is a method of treating, preventing, or managing (PD-L1 negative) bladder cancer in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is Bacillus Calmette-Guérin (BCG).

In one embodiment, provided herein is a method of treating, preventing, or managing (PD-L1 negative) head and neck cancer in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is cetuximab, nimotuzumab, bevacizumab, or erlotinib.

In one embodiment, provided herein is a method of treating, preventing, or managing (PD-L1 negative) breast cancer (e.g., triple-negative breast cancer) in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is bevacizumab or trastuzumab.

In one embodiment, provided herein is a method of treating, preventing, or managing (PD-L1 negative) ovarian cancer in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is bevacizumab.

In one embodiment, provided herein is a method of treating, preventing, or managing (PD-L1 negative) colon cancer in a subject, comprising administering to the subject a therapeutically effective amount of a compound provided herein (e.g., Compound 1), or a pharmaceutically acceptable form thereof, wherein the subject has been pre-treated or previously treated with one or more immunotherapy treatments. In one embodiment, the immunotherapy treatment is bevacizumab, cetuximab, or panitumumab.

In one embodiment, the subject is a human. In one embodiment, the subject is a (human) patient. In one embodiment, the subject is identified as having or being at risk of having a PI3K-gamma mediated disorder via the use of a biomarker.

In one embodiment, the subject has high-circulating myeloid-derived suppressor cells. In one embodiment, “high” MDSC level means it is higher than a reference level. In one embodiment, high-circulating MDSCs means MDSCs≥20.5% as measured by CLIA-certified Serametrix assay. In one embodiment, circulating mMDSC levels are measured in baseline peripheral blood samples based on a Clinical Laboratory Improvement Amendments (CLIA)-certified flow cytometry assay (low [<22.3%] or high [≥22.3%]. In one embodiment, the reference level is the median MDSC level of a patient population (e.g., the patient population in a clinical trial example provided herein).

In one embodiment, the methods further comprise administering to the subject a therapeutically effective amount of a second agent.

In one embodiment, the second agent used in the methods provided herein is an immunomodulatory.

In one embodiment, the immunomodulator is an immune checkpoint therapy, e.g., an immune checkpoint therapy chosen from an inhibitor of PD-1, PD-L1, PD-L2, CTLA4, TIM3, LAG3, VISTA, BTLA, TIGIT, LAIR1, CD160, 2B4, TGFR-beta, or IDO/TDO, or any combination thereof. In one embodiment, the immune checkpoint therapy is an inhibitor of CTLA-4, PD-1, or PD-L1. The immune checkpoint therapy can be chosen from an antibody or fragment thereof, an inhibitory nucleic acid, a soluble ligand, or a fusion of an immune checkpoint therapy (e.g., CTLA-4, PD-1, or PD-1 ligand) with a Fc region of an immunoglobulin.

In certain embodiments, the immunomodulator is an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

The certain embodiments, the immunomodulator is chosen from a costimulatory ligand, a MCSF/CSF-1R inhibitor, an immunostimulant, a CXCR4/CXCL12 inhibitor, a CCL2 inhibitor, or a CCR2 inhibitor.

In certain embodiments, the PI3K gamma inhibitor or a compound as described herein and the immunomodulator are in a single dosage form. In certain embodiments, the PI3K gamma inhibitor or a compound as described herein and the immunomodulator are in separate dosage forms. In certain embodiments, the PI3K gamma inhibitor or a compound as described herein and the immunomodulator are administered concurrently. In certain embodiments, the PI3K gamma inhibitor or a compound as described herein is administered subsequent to the immunomodulator. In certain embodiments, the PI3K gamma inhibitor or a compound as described herein is administered prior to the immunomodulator.

In certain embodiments, the effective amount of the PI3K gamma inhibitor or a compound as described herein, the immunomodulator, or both that is an amount sufficient to cause a decrease in tumor growth of at least 10%, 20%, 30%, 40%, or 50% compared to a reference value, is reduced.

In certain embodiments, the subject has, or is determined to have, a decrease in tumor growth of at least 10%, 20%, 30%, 40%, or 50% compared to a reference value, after administration of the PI3K gamma inhibitor or the compound.

In one embodiment, the second agent is an immune checkpoint therapy.

There are two main types of immune checkpoint therapies: an activator of a costimulatory molecule, and an inhibitor of an immune checkpoint molecule.

When the immune checkpoint therapy is an activator of a costimulatory molecule, it may be, e.g., chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand. In certain embodiments, the immune checkpoint therapy is an inhibitor of OX40 or anti-OX40 ab.

In the second situation, the immune checkpoint therapy is an inhibitor of an immune checkpoint molecule, for instance, an inhibitor of PD-1, PD-L1, PD-L2, CTLA4, TIM3, LAG3, VISTA, BTLA, TIGIT, LAIR1, CD160, 2B4 and/or TGFR beta. For instance, the inhibitor of an immune checkpoint molecule may inhibit PD-1, PD-L1, LAG-3, TIM-3 or CTLA4, or any combination thereof.

Inhibition of an inhibitory molecule can be performed at the DNA, RNA or protein level. For example, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

The antibody molecule may be, e.g., a full antibody or fragment thereof (e.g., a Fab, F(ab′)₂, Fv, or a single chain Fv fragment (scFv)). The antibody molecule may be, e.g., in the form of a bispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In certain embodiments, the antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

In certain embodiments, the immune checkpoint therapy is an inhibitor of PD-1, e.g., human PD-1. In another embodiment, the immune checkpoint therapy is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immune checkpoint therapies, e.g., in combination with an inhibitor of LAG-3, TIM-3 or CTLA4. In some embodiments, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. Other combinations of immune checkpoint therapies with a PD-1 inhibitor (e.g., one or more of PD-L2, CTLA4, TIM3, LAG3, VISTA, BTLA, TIGIT, LAIRI, CD160, 2B4 and/or TGFR) are also within the present invention. Any of the PI3K inhibitor molecules known in the art or disclosed herein can be used in the aforesaid combinations of inhibitors of checkpoint molecule.

In one embodiment, the immune checkpoint therapy is a PD-1 inhibitor. In one embodiment, the PD-1 inhibitor is nivolumab, pembrolizumab, pidilizumab, AMP-244, or AMP-514. In one embodiment, the PD-1 inhibitor is nivolumab. In one embodiment, the PD-1 inhibitor is pembrolizumab.

In some embodiments, the anti-PD-1 antibody is nivolumab. Alternative names for nivolumab include MDX-1106, MDX-1106-04, ONO-4538, or BMS-936558. In some embodiments, the anti-PD-1 antibody is nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168.

In one embodiment, nivolumab is administered according to a dose and route approved by FDA or equivalent agency. In one embodiment, nivolumab is administered intravenously at from about 120 mg to about 360 mg every 2 weeks (e.g., day 1 of each 14-day cycle, or days land 15 of each 28-day cycle). In one embodiment, nivolumab is administered intravenously at about 240 mg every 2 weeks. In one embodiment, nivolumab is administered intravenously at from about 240 mg to about 480 mg every 3 weeks (e.g., day 1 of each 21-day cycle). In one embodiment, nivolumab is administered intravenously at about 360 mg every 3 weeks. In one embodiment, nivolumab is administered intravenously at from about 360 mg to about 600 mg every 4 weeks (e.g., day 1 of each 28-day cycle). In one embodiment, nivolumab is administered intravenously at about 480 mg every 4 weeks.

In other embodiments, the anti-PD-1 antibody is pembrolizumab. Pembrolizumab (Trade name KEYTRUDA formerly lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, WO2009/114335, and U.S. Pat. No. 8,354,509.

In some embodiments, the anti-PD-1 antibody is pidilizumab. Pidilizumab (CT-011; Cure Tech) is a humanized IgG1k monoclonal antibody that binds to PD1. Pidilizumab and other humanized anti-PD-1 monoclonal antibodies are disclosed in WO2009/101611. Other anti-PD1 antibodies are disclosed in U.S. Pat. No. 8,609,089, US 2010028330, and/or US 20120114649. Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 inhibitor is AMP-224. In some embodiments, a PI3K inhibitor, e.g., a PI3K-γ inhibitor as described herein (e.g., Compound 1), is administered together with an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the combination therapy is used in a method of treating a cancer, as described herein.

In one embodiment, the immune checkpoint therapy is a PD-L1 inhibitor. In one embodiment, the PD-L1 inhibitor is atezolizumab (MPDL3280A), YW243.55.S70, MSB0010718C, MDX-1105, or MEDI-4736.

In one embodiment, the PD-L1 inhibitor is atezolizumab. In one embodiment, atezolizumab is administered according to a dose and route approved by FDA or equivalent agency. In one embodiment, atezolizumab is administered intravenously at from about 480 mg to about 1200 mg every 2 weeks (e.g., day 1 of each 14-day cycle, or days land 15 of each 28-day cycle). In one embodiment, atezolizumab is administered intravenously at about 840 mg every 2 weeks. In one embodiment, atezolizumab is administered intravenously at from about 840 mg to about 1680 mg every 3 weeks (e.g., day 1 of each 21-day cycle). In one embodiment, atezolizumab is administered intravenously at about 1200 mg every 3 weeks. In one embodiment, atezolizumab is administered intravenously at from about 1200 mg to about 2160 mg every 4 weeks (e.g., day 1 of each 28-day cycle). In one embodiment, atezolizumab is administered intravenously at about 1680 mg every 4 weeks.

In some embodiments, the PD-L1 inhibitor is anti-PD-LI antibody. In some embodiments, the anti-PD-L1 inhibitor is chosen from YW243.55.S70, MPDL3280A, MEDI-4736, MSB-0010718C, or MDX-1105.

In one embodiment, the PD-L1 inhibitor is MDX-1105. MDX-1105, also known as BMS-936559, is an anti-PD-LI antibody described in WO2007/005874.

In one embodiment, the PD-L1 inhibitor is YW243.55.S70. The YW243.55.S70 antibody is an anti-PD-LI described in WO 2010/077634 (heavy and light chain variable region sequences shown in SEQ ID Nos. 20 and 21, respectively).

In one embodiment, the PD-L1 inhibitor is MPDL3280A (Genentech/Roche). MPDL3280A is a human Fc optimized IgG1 monoclonal antibody that binds to PD-L1. MPDL3280A and other human monoclonal antibodies to PD-L1 are disclosed in U.S. Pat. No. 7,943,743 and U.S Publication No.: 20120039906. Other anti-PD-L1 binding agents include MDX-1105 (also referred to as BMS-936559, and, e.g., anti-PD-L1 binding agents disclosed in WO2007/005874).

In some embodiments, the anti-PD-LI binding antagonist is chosen from YW243.55.S70, MPDL3280A, MEDI-4736, MSB-0010718C, or MDX-1105. MDX-1105, also known as BMS-936559, is an anti-PD-LI antibody described in WO2007/005874. Antibody YW243.55.S70 (heavy and light chain variable region sequences shown in SEQ ID Nos. 20 and 21, respectively) is an anti-PD-LI described in WO 2010/077634.

In some embodiments, the anti-PD-L1 antibody is MSB0010718C. MSB0010718C (also referred to as A09-246-2; Merck Serono) is a monoclonal antibody that binds to PD-L1.

In some embodiments, the immune checkpoint therapy inhibits CTLA-4, PD-1, or PD-L1, or any combination thereof. The immune checkpoint therapy may be, e.g., a small molecule or an antibody. In some embodiments, the immune checkpoint therapy is an antibody that inhibits programmed cell death 1 (also known as PD-1). In another embodiment, the immune checkpoint therapy is nivolumab (also known as Opdivo). In some embodiments, the immune checkpoint therapy is anti-PD-L1 (programmed cell death ligand 1, also known as cluster of differentiation 274 (CD274)), anti-PD-L2, or anti-CTLA-4 (cytotoxic T-lymphocyte antigen 4, also known as cluster of differentiation (CD152)) antibody. Certain anti-PD-1, anti-PD-L1, and anti-CTLA-4 antibodies have activity in preclinical and clinical tumor models. Cancer Res; 73(12) Jun. 15, 2013; Curran M A et al. PNAS 2010; 107:4275-4280; Topalian et al. N Engl J Med 2012; 366:2443-2454; Wolchok et al., 2013. NEJM 369.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in WO2010/027827 and WO2011/066342).

In one embodiment, the LAG-3 inhibitor is an anti-LAG-3 antibody molecule. In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218.

In certain embodiments, the combination therapies disclosed herein include a modulator of a costimulatory molecule or an inhibitory molecule, e.g., a co-inhibitory ligand or receptor.

In one embodiment, the costimulatory modulator, e.g., agonist, of a costimulatory molecule is chosen from an agonist (e.g., an agonistic antibody or antigen-binding fragment thereof, or soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In another embodiment, the combination therapies disclosed herein include a costimulatory molecule, e.g., an agonist associated with a positive signal that includes a costimulatory domain of CD28, CD27, ICOS and GITR.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.:WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, the inhibitor is a soluble ligand (e.g., a CTLA-4-Ig), or an antibody or antibody fragment that binds to PD-L1, PD-L2 or CTLA4. For example, a compound disclosed herein, e.g., Compound 1, can be administered in combination with an anti-CTLA-4 antibody, e.g., ipilimumab, for example, to treat a cancer (e.g., a cancer chosen from: a melanoma, e.g., a metastatic melanoma; a lung cancer, e.g., a non-small cell lung carcinoma; or a prostate cancer). Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, Yervoy, CAS No. 477202-00-9). In some embodiments, a compound provided herein is administered in combination with an anti-PD-L1 inhibitor (e.g., nivolumab) and a CTLA-4 antibody (e.g., ipilimumab). In some embodiments, a compound provided herein is administered in combination with nivolumab and ipilimumab.

In some embodiments, a compound provided herein, or a pharmaceutically acceptable derivative (e.g., salt or solvate) thereof, is administered in combination with an anti-PD-L1 or anti-CTLA-4 antibody. In some embodiments, a compound provided herein, or a pharmaceutically acceptable derivative (e.g., salt or solvate) thereof, is administered in combination with an anti-PD-L1 antibody. In another embodiment, a compound provided herein, or a pharmaceutically acceptable derivative (e.g., salt or solvate) thereof, is administered in combination with anti-CTLA-4 antibody. In some embodiments, the anti-PD-L1 antibody is selected from BMS-936559, MPDL3280A, and MDX-1105. In some embodiments, the anti-CTLA-4 antibody is selected from ipilimumab and tremelimumab.

While not wishing to be bound by theory, it is believed that tumor growth is influenced by at least two classes of immune cells in the tumor microenvironment: effector cells (including cytotoxic cells and M1 macrophages) which have anti-tumor activity, and tumor associated suppressor cells (including M2 macrophages, MDSC, Tregs, and regulatory dendritic cells) which have pro-tumor activity because they inhibit the effector cells or provide direct growth stimulation to the tumor cells or tumor vasculature. An abundance of suppressor cells can lead to tumor immune tolerance, and enhancement of tumor growth. A combination cancer therapy can be designed taking this mechanism into consideration.

In some embodiments, a compound provided herein, or a pharmaceutically acceptable derivative (e.g., salt or solvate) thereof, is administered in combination with one or more immune checkpoint therapies. In some embodiments, provided herein is a method of treating a (PD-L1 negative) cancer in a subject, comprising administering to the subject a PI3K gamma inhibitor or a compound as described herein (e.g., Compound 1) in combination with one or more immune checkpoint therapies (e.g., PD-1 or PD-L1 inhibitors). In some embodiments, provided herein is a method of treating a solid cancer in a subject, comprising administering to the subject Compound 1, or a pharmaceutically acceptable form thereof, in combination with one or more of PD-1 or PD-L1 inhibitors. In one embodiment, the cancer is melanoma, lung cancer (e.g., non-small cell lung cancer), head and neck cancer (e.g., head and neck squamous cell carcinoma), renal cell carcinoma, bladder cancer, gallbladder carcinoma, breast cancer (e.g., triple negative breast cancer), colon cancer, glioblastoma, adrenocortical carcinoma, mesothelioma, colorectal cancer, ovarian cancer, endometrial cancer, or urothelial carcinoma.

In some embodiments, the subject is naive to immunotherapy treatment. In some embodiments, the subject is naive to radiation therapy treatment. In some embodiments, the subject is naive to chemotherapy treatment.

In one embodiment, the solid tumor patient is naive to immune therapy (e.g., immune checkpoint therapy, e.g., anti-PD-1 and/or anti-PD-L1 therapy). In one embodiment, the solid tumor patient has been previously treated with one or more (e.g., two or more) immune therapy (e.g., immune checkpoint therapy, e.g., anti-PD-1 and/or anti-PD-L1 therapy).

In one embodiment, the solid tumor is breast cancer. In one embodiment, the breast cancer is triple negative breast cancer. In one embodiment, the breast cancer is the breast cancer is unresectable locally advanced or metastatic triple negative breast cancer. In one embodiment, the breast cancer (e.g., triple negative breast cancer) patient is naive to immune therapy (e.g., immune checkpoint therapy, e.g., anti-PD-1 and/or anti-PD-L1 therapy). In one embodiment, the breast cancer (e.g., triple negative breast cancer) patient has been previously treated with one or more (e.g., two or more) immune therapy (e.g., immune checkpoint therapy, e.g., anti-PD-1 and/or anti-PD-L1 therapy).

In one embodiment, the solid tumor is head and neck cancer. In one embodiment, the head and neck cancer is head and neck squamous cell carcinoma. In one embodiment, the head and neck cancer (e.g., head and neck squamous cell carcinoma) patient is naive to immune therapy (e.g., immune checkpoint therapy, e.g., anti-PD-1 and/or anti-PD-L1 therapy). In one embodiment, the head and neck cancer (e.g., head and neck squamous cell carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is lung cancer. In one embodiment, the lung cancer is non-small cell lung cancer. In one embodiment, the lung cancer (e.g., non-small cell lung cancer) patient is naive to immune therapy (e.g., immune checkpoint therapy, e.g., anti-PD-1 and/or anti-PD-L1 therapy). In one embodiment, the lung cancer (e.g., non-small cell lung cancer) patient has been previously treated with one or more (e.g., two or more) immune therapy (e.g., immune checkpoint therapy, e.g., anti-PD-1 and/or anti-PD-L1 therapy).

In one embodiment, the solid tumor is melanoma. In one embodiment, the melanoma patient is naive to immune therapy (e.g., immune checkpoint therapy, e.g., anti-PD-1 and/or anti-PD-L1 therapy). In one embodiment, the melanoma patient has been previously treated with one or more (e.g., two or more) immune therapy (e.g., immune checkpoint therapy, e.g., anti-PD-1 and/or anti-PD-L1 therapy).

In one embodiment, the solid tumor is colon cancer. In one embodiment, the colon cancer patient is naive to immune therapy (e.g., immune checkpoint therapy, e.g., anti-PD-1 and/or anti-PD-L1 therapy). In one embodiment, the colon cancer patient has been previously treated with one or more (e.g., two or more) immune therapy (e.g., immune checkpoint therapy, e.g., anti-PD-1 and/or anti-PD-L1 therapy).

In one embodiment, the solid tumor is glioblastoma. In one embodiment, the glioblastoma patient is naive to immune therapy (e.g., immune checkpoint therapy, e.g., anti-PD-1 and/or anti-PD-L1 therapy). In one embodiment, the glioblastoma patient has been previously treated with one or more (e.g., two or more) immune therapy (e.g., immune checkpoint therapy, e.g., anti-PD-1 and/or anti-PD-L1 therapy).

In one embodiment, the solid tumor is renal cell carcinoma. In one embodiment, the renal cell carcinoma is clear cell renal cell carcinoma. In one embodiment, the renal cell carcinoma (e.g., clear cell renal cell carcinoma) patient is naive to immune therapy (e.g., immune checkpoint therapy, e.g., anti-PD-1 and/or anti-PD-L1 therapy). In one embodiment, the renal cell carcinoma (e.g., clear cell renal cell carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is gallbladder carcinoma. In one embodiment, the gallbladder carcinoma is microsatellite-stable gallbladder carcinoma. In one embodiment, the gallbladder carcinoma (e.g., microsatellite-stable gallbladder carcinoma) patient is naive to immune therapy (e.g., immune checkpoint therapy, e.g., anti-PD-1 and/or anti-PD-L1 therapy). In one embodiment, the gallbladder carcinoma (e.g., microsatellite-stable gallbladder carcinoma) patient has been previously treated with one or more (e.g., two or more) immune therapy (e.g., immune checkpoint therapy, e.g., anti-PD-1 and/or anti-PD-L1 therapy).

In one embodiment, the solid tumor is adrenocortical carcinoma. In one embodiment, the adrenocortical carcinoma patient is naive to immune therapy (e.g., immune checkpoint therapy, e.g., anti-PD-1 and/or anti-PD-L1 therapy). In one embodiment, the adrenocortical carcinoma patient has been previously treated with one or more (e.g., two or more) immune therapy (e.g., immune checkpoint therapy, e.g., anti-PD-1 and/or anti-PD-L1 therapy).

In one embodiment, the solid tumor is mesothelioma. In one embodiment, the mesothelioma is epithelioid mesothelioma, sarcomatoid mesothelioma, or biphasic mesothelioma. In one embodiment, the mesothelioma patient is naive to immune therapy (e.g., immune checkpoint therapy, e.g., anti-PD-1 and/or anti-PD-L1 therapy). In one embodiment, the mesothelioma patient has been previously treated with one or more (e.g., two or more) immune therapy (e.g., immune checkpoint therapy, e.g., anti-PD-1 and/or anti-PD-L1 therapy).

In one embodiment, the solid tumor is colorectal cancer. In one embodiment, the colorectal cancer patient is naive to immune therapy (e.g., immune checkpoint therapy, e.g., anti-PD-1 and/or anti-PD-L1 therapy). In one embodiment, the colorectal cancer patient has been previously treated with one or more (e.g., two or more) immune therapy (e.g., immune checkpoint therapy, e.g., anti-PD-1 and/or anti-PD-L1 therapy).

In one embodiment, the solid tumor is ovarian cancer. In one embodiment, the ovarian cancer patient is naive to immune therapy (e.g., immune checkpoint therapy, e.g., anti-PD-1 and/or anti-PD-L1 therapy). In one embodiment, the ovarian cancer patient has been previously treated with one or more (e.g., two or more) immune therapy (e.g., immune checkpoint therapy, e.g., anti-PD-1 and/or anti-PD-L1 therapy).

In one embodiment, the solid tumor is endometrial cancer. In one embodiment, the endometrial cancer patient is naive to immune therapy (e.g., immune checkpoint therapy, e.g., anti-PD-1 and/or anti-PD-L1 therapy). In one embodiment, the endometrial cancer patient has been previously treated with one or more (e.g., two or more) immune therapy (e.g., immune checkpoint therapy, e.g., anti-PD-1 and/or anti-PD-L1 therapy).

In one embodiment, the solid tumor is urothelial carcinoma. In one embodiment, the urothelial carcinoma patient is naive to immune therapy (e.g., immune checkpoint therapy, e.g., anti-PD-1 and/or anti-PD-L1 therapy). In one embodiment, the urothelial carcinoma patient has been previously treated with one or more (e.g., two or more) immune therapy (e.g., immune checkpoint therapy, e.g., anti-PD-1 and/or anti-PD-L1 therapy).

In some embodiments, the subject has been pre-treated or previously treated with one or more immunotherapy treatments. In one embodiment, the subject is responsive to the pre-treatment or previous treatment with the immunotherapy. In one embodiment, the immunotherapy treatment is a checkpoint treatment such as a PD-1 or PD-L1 inhibitor. In one embodiment, the subject is a smoker.

In one embodiment, the cancer is melanoma, and the subject has been pre-treated or previously treated with one or more immunotherapy treatments. In one embodiment, the subject has been pre-treated or previously treated with two or more immunotherapy treatments.

In one embodiment, the cancer is head and neck cancer, lung cancer (e.g., non-small cell lung cancer), renal cell carcinoma, or bladder cancer, and the subject has been pre-treated or previously treated with one immunotherapy treatment.

In one embodiment, the cancer is breast cancer (e.g., triple-negative breast cancer), ovarian cancer, glioblastoma, or colon cancer, and the subject is naive to immunotherapy treatment.

In some embodiments, provided herein is a method of treating (PD-L1 negative) melanoma, lung cancer (e.g., non-small cell lung cancer), head and neck cancer (e.g., head and neck squamous cell carcinoma), renal cell carcinoma, bladder cancer, gallbladder carcinoma, breast cancer (e.g., triple negative breast cancer), colon cancer, glioblastoma, adrenocortical carcinoma, mesothelioma, colorectal cancer, ovarian cancer, endometrial cancer, or urothelial carcinoma, comprising administering to a patient a therapeutically effective amount of a compound provided herein (e.g., Compound 1), or a pharmaceutically acceptable derivative (e.g., salt or solvate) thereof, in combination with an anti-PD-L1 or an anti-CTLA-4 antibody. In another embodiment, the cancer is chosen form a carcinoma (e.g., advanced or metastatic carcinoma), melanoma or a lung carcinoma, e.g., a non-small cell lung carcinoma. In one embodiment, the cancer is a lung cancer, e.g., a non-small cell lung cancer. In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600E mutation). In another embodiment, the cancer is a hepatocarcinoma, e.g., an advanced hepatocarcinoma, with or without a viral infection, e.g., a chronic viral hepatitis. In another embodiment, the cancer is a prostate cancer, e.g., an advanced prostate cancer. In yet another embodiment, the cancer is a myeloma, e.g., multiple myeloma. In yet another embodiment, the cancer is a renal cancer, e.g., a renal cell carcinoma (RCC) (e.g., a metastatic RCC or clear cell renal cell carcinoma (CCRCC)).

For example, a compound provided herein (e.g., Compound 1) can be administered in combination with an anti-CTLA-4 antibody, e.g., ipilimumab, for example, to treat a cancer (e.g., a cancer chosen from: a melanoma, e.g., a metastatic melanoma; a lung cancer, e.g., a non-small cell lung carcinoma; or a prostate cancer). In one embodiment, a compound provided herein (e.g., Compound 1) is administered after treatment with an anti-CTLA4 antibody (e.g., ipilimumab) with or without a BRAF inhibitor (e.g., vemurafenib or dabrafenib).

In some embodiments, the immune checkpoint therapy is a costimulatory ligand. In some embodiments, the costimulatory ligand is OX40L, 41BBL, CD153, ICOSL, or CD40L.

In some embodiments, the immune checkpoint therapy is a MCSF/CSF-1R inhibitor. An anti-CSF-1R can deplete TAMs, resulting in tumor growth inhibition. Cancer Cell 25, 1-14, Jun. 16, 2014. In some embodiments, the CSF-1R inhibitor is BLZ945, GW2850, R05509554, or PLX3397. In some embodiments, the CSF-1R inhibitor is BLZ945 or GW2850. In some embodiments, the CSF-1R inhibitor is PLX3397.

In some embodiments, the immune checkpoint therapy is an immunostimulant. In some embodiments, the immunostimulant is GMCSF, TLR ligands, 41BBL, or ICOSL. In some embodiments, the immune checkpoint therapy is a CXCR4/CXCL12 inhibitor. In some embodiments, the CXCR4/CXCL12 inhibitor is AMD3100, AMD11070, AMD12118, AMD11814, or AMD13073. In some embodiments, the CXCR4/CXCL12 inhibitor is AMD3100.

In some embodiments, the immunotherapy is a CCL2 and/or CCR2 antagonist. In some embodiments, the antagonist of CCL2 and/or CCR2 is an anti-CCL2 or CCR2 antibody. CCL2 is a chemokine and CCR2 is a chemokine receptor. CCL2 and CCR2, according to non-limiting theory, play a role in MDSC migration.

In some embodiments, a PI3K-γ inhibitor disclosed herein, e.g., Compound 1, is administered in combination with an IDO (indoleamine 2,3-dioxygenase) inhibitor or an TDO (tryptophan 2,3-dioxygenase) inhibitor. In one embodiment, the IDO inhibitor is indoximod, NLG919, INCB024360, F001287, norharmane, rosmarinic acid, or alpha-methyl-tryptophan. Although IDO inhibitors act within the TME, they do not specifically target MDSCs. The overexpression of IDO by dendritic cells creates an immunosuppressive tumor microenvironment.

In some embodiment, a PI3K-γ inhibitor disclosed herein, e.g., Compound 1, is administered to a subject concurrent or prior to the administration of immune checkpoint therapy. In some embodiment, an immunostimulant is administered to a subject concurrent or prior to the administration of immune checkpoint therapy. In some embodiment, chemotherapy (e.g., carboplatin, oxaliplatin, or radiation) is administered to a subject concurrent or prior to the administration of immune checkpoint therapy.

In some embodiments, a PI3K-γ inhibitor disclosed herein, e.g., Compound 1, is administered in combination with an ARG1 inhibitor. While not wishing to be bound by theory, it has been reported that tumor associated myeloid cells establish an immunosuppressive microenvironment in tumors through the expression of Arginase-1, which depletes the tumor microenvironment of arginine, thereby the death or inhibition of anti-tumor immune cells. Schmid et al., Proceedings: AACR 103rd Annual Meeting 2012, Cancer Research: Apr. 15, 2012; Volume 72, Issue 8, Supplement 1. It has been reported that suppression of PI3Kgamma or Arginase-1 expression blocked myeloid cell induced death of T cells in vitro. Id. According to the non-limiting theory, PI3Kgamma inhibition blocks Arginase-1 expression, thereby increasing the number of CD8+ T cells in tumors, stimulating T cell-mediated cytotoxicity of tumor cells, and suppressing growth and metastasis of tumors. Combination therapies can be designed in accordance with this mechanism.

The PI3K γ inhibitors disclosed herein can have minimal effects on T-cell activation when compared to the suppressive effect of a PI3K δ inhibitor on T-cell activation. Lewis lung carcinoma tumor growth can be reduced in PI3K γ knockout mice and can have decreased tumor associated suppressive myeloid cell infiltrates. Tumor associated suppressive myeloid cells can include e.g., myeloid derived suppressor cells (MDSCs) and tumor associated macrophages (TAMs). PI3K γ knockout mice have TAMs where the M2 phenotype is lost. M2 cells are immunosuppressive and support tumor growth. PI3K inhibitors provided herein can block M2 phenotype (e.g., in an in vitro differentiation system), and thus can slow tumor growth.

For example, the effect of PI3K γ inhibitors and PI3K δ inhibitors on T cell activation as measured by inhibition of IFN-γ in response to ConA has shown that PI3K-δ is plays a role in mediating T cell activation, while PI3K-γ has minimal effects on T-cell activation. The IC₅₀ for a PI3K δ inhibitor in this assay is 3 nM, and the IC₅₀ for a PI3K γ inhibitor is 2500 nM. Administration of PI1K-γ inhibitors can lead to impaired T-cell migration but may have reduced effects on T-cell proliferation or activation.

In some embodiments, the PI3K γ inhibitors disclosed herein can have potent effects on tumor associated suppressive myeloid cells without inhibiting the effector T-cell. The PI3K γ inhibitors disclosed herein can have potent effects on tumor associated suppressive myeloid cells without blocking anti-tumor T-cell effects and thus can increase T cell activity. In one embodiment, this effect can be enhanced by administering CTLA4 antagonists and/or PD-1 and PD-L1 antagonists. The PI3K γ inhibitors disclosed herein can increase T cell activation and proliferation. In some embodiments, provided herein is a method of blocking tumor associated suppressive myeloid cells without inhibiting the effects on anti-tumor T-cells comprising administering an effective amount of a PI3K γ inhibitor disclosed herein or a pharmaceutically acceptable salt thereof to a subject. In some embodiments, provided herein is a method of blocking tumor associated suppressive myeloid cells without inhibiting the effects on anti-tumor T-cells comprising administering an effective amount of a compound disclosed herein or a pharmaceutically acceptable salt thereof to a subject. In some embodiments, the subject has lung cancer, breast cancer, glioblastoma, or lymphoma (e.g., non-Hodgkin's lymphoma).

Further provided herein are methods of modulating kinase activity by contacting a kinase with an amount of a compound provided herein sufficient to modulate the activity of the kinase. Modulate can be inhibiting or activating kinase activity. In some embodiments, provided herein are methods of inhibiting kinase activity by contacting a kinase with an amount of a compound provided herein sufficient to inhibit the activity of the kinase. In some embodiments, provided herein are methods of inhibiting kinase activity in a solution by contacting said solution with an amount of a compound provided herein sufficient to inhibit the activity of the kinase in said solution. In some embodiments, provided herein are methods of inhibiting kinase activity in a cell by contacting said cell with an amount of a compound provided herein sufficient to inhibit the activity of the kinase in said cell. In some embodiments, provided herein are methods of inhibiting kinase activity in a tissue by contacting said tissue with an amount of a compound provided herein sufficient to inhibit the activity of the kinase in said tissue. In some embodiments, provided herein are methods of inhibiting kinase activity in an organism by contacting said organism with an amount of a compound provided herein sufficient to inhibit the activity of the kinase in said organism. In some embodiments, provided herein are methods of inhibiting kinase activity in an animal by contacting said animal with an amount of a compound provided herein sufficient to inhibit the activity of the kinase in said animal. In some embodiments, provided herein are methods of inhibiting kinase activity in a mammal by contacting said mammal with an amount of a compound provided herein sufficient to inhibit the activity of the kinase in said mammal. In some embodiments, provided herein are methods of inhibiting kinase activity in a human by contacting said human with an amount of a compound provided herein sufficient to inhibit the activity of the kinase in said human. In some embodiments, the % of kinase activity after contacting a kinase with a compound provided herein is less than 1, 5, 10, 20, 30, 40, 50, 60, 70, 80 90, 95, or 99% of the kinase activity in the absence of said contacting step.

In one embodiment, the methods further comprise administering to the subject a therapeutically effective amount of a third agent.

In one embodiment, the third agent is paclitaxel. In one embodiment, the third agent is nab-paclitaxel (e.g., abraxane). In one embodiment, the third agent is nanoparticle albumin-bound paclitaxel. Nab-paclitaxel is a nanoparticle albumin-bound formulation of paclitaxel (Taxol), a mitotic inhibitor chemotherapy, with less toxicity than solvent-based paclitaxel and achieves a 33% higher tumor uptake in preclinical models.

In one embodiment, nab-paclitaxel is administered according to a dose and route approved by FDA or equivalent agency. In one embodiment, nab-paclitaxel is administered intravenously at from about 130 mg/m² to about 390 mg/m² intravenously over every 3 weeks. In one embodiment, nab-paclitaxel is administered intravenously at about 260 mg/m² intravenously over every 3 weeks. In one embodiment, nab-paclitaxel is administered intravenously at from about 50 mg/m² to about 150 mg/m² on days 1, 8, and 15 of each 21-day cycle. In one embodiment, nab-paclitaxel is administered intravenously at about 100 mg/m² on days 1, 8, and 15 of each 21-day cycle. In one embodiment, nab-paclitaxel is administered intravenously at from about 50 mg/m² to about 150 mg/m² on days 1, 8 and 15 of each 28-day cycle. In one embodiment, nab-paclitaxel is administered intravenously at about 100 mg/m² on days 1, 8 and 15 of each 28-day cycle. In one embodiment, nab-paclitaxel is administered intravenously at about 125 mg/m² intravenously on days 1, 8, and 15 of each 28-day cycle. In one embodiment, the intravenous administration of nab-paclitaxel is over about 30-40 minutes. In one embodiment, the intravenous administration of nab-paclitaxel is over about 30 minutes.

In one embodiment, the third agent is bevacizumab. Bevacizumab is an anti-vascular endothelial growth factor (anti-VEGF) recombinant monoclonal antibody that is approved by the FDA for the treatment of multiple solid tumors in combination with chemotherapy.

In one embodiment, bevacizumab is administered according to a dose and route approved by FDA or equivalent agency. In one embodiment, bevacizumab is administered intravenously at from about 2.5 mg/kg to about 15 mg/kg every 2 weeks (e.g., on day 1 of each 14-day cycle). In one embodiment, bevacizumab is administered intravenously at about 5 mg/kg every 2 weeks. In one embodiment, bevacizumab is administered intravenously at about 10 mg/kg every 2 weeks. In one embodiment, bevacizumab is administered intravenously at from about 10 mg/kg to about 20 mg/kg every 3 weeks (e.g., on day 1 of each 21-day cycle). In one embodiment, bevacizumab is administered intravenously at about 15 mg/kg every 3 weeks.

In one embodiment, provided herein is a method for treating breast cancer in a subject, comprising administering to the subject a therapeutically effective amount of a compound of the formula:

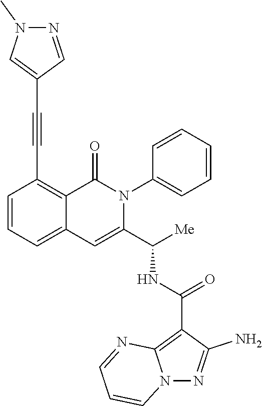 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor, wherein the breast cancer is PD-L1 negative.

In one embodiment, provided herein is a method for treating breast cancer in a subject, comprising: (i) identifying the breast cancer in the subject to be PD-L1 negative, and (ii) administering to the subject a therapeutically effective amount of a compound of the formula:

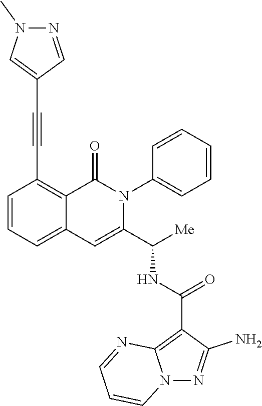 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor.

In one embodiment, the breast cancer is triple negative breast cancer. In one embodiment, the breast cancer (e.g., triple negative breast cancer) patient is naive to immune therapy (e.g., immune checkpoint therapy, e.g., anti-PD-1 and/or anti-PD-L1 therapy). In one embodiment, the breast cancer (e.g., triple negative breast cancer) patient has been previously treated with one or more (e.g., two or more) immune therapy (e.g., immune checkpoint therapy, e.g., anti-PD-1 and/or anti-PD-L1 therapy).

In one embodiment, the breast cancer is locally advanced and/or metastatic. In one embodiment, the breast cancer is advanced. In one embodiment, the breast cancer is locally advanced. In one embodiment, the breast cancer is metastatic. In one embodiment, the breast cancer is unresectable. In one embodiment, the breast cancer is unresectable locally advanced or metastatic triple negative breast cancer.

In one embodiment, the PD-L1 inhibitor is atezolizumab, YW243.55.S70, MSB0010718C, MDX-1105, or MEDI-4736. In one embodiment, the PD-L1 inhibitor is atezolizumab. In one embodiment, atezolizumab is administered intravenously at about 840 mg every 2 weeks. In one embodiment, atezolizumab is administered intravenously at a dose of about 840 mg on days 1 and 15 of one or more 28-day cycles.

In one embodiment, the administration of the compound and PD-L1 inhibitor is further in combination with paclitaxel. In one embodiment, the administration of the compound and PD-L1 inhibitor is further in combination with nab-paclitaxel (e.g., abraxane). In one embodiment, the administration of the compound and PD-L1 inhibitor is further in combination with nanoparticle albumin-bound paclitaxel. In one embodiment, nab-paclitaxel is administered intravenously at a dose of about 100 mg/m² on days 1, 8, and 15 of one or more 28-day cycles.

In one embodiment, the method is for treating breast cancer as front-line treatment. In one embodiment, the method is for treating breast cancer as first-line treatment.

In one embodiment, the method is for treating (PD-L1 negative) triple negative breast cancer with Compound 1, in combination with atezolizumab and nab-paclitaxel.

In one embodiment, provided herein is a method for treating renal cell carcinoma in a subject, comprising: administering to the subject a therapeutically effective amount of a compound of the formula:

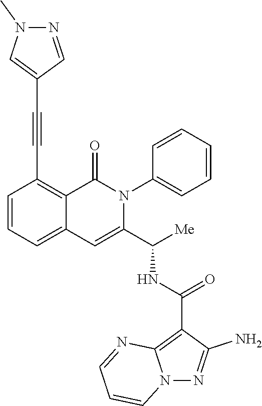 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor, wherein the renal cell carcinoma is PD-L1 negative.

In one embodiment, provided herein is a method for treating renal cell carcinoma in a subject, comprising: (i) identifying the renal cell carcinoma in the subject to be PD-L1 negative, and (ii) administering to the subject a therapeutically effective amount of a compound of the formula:

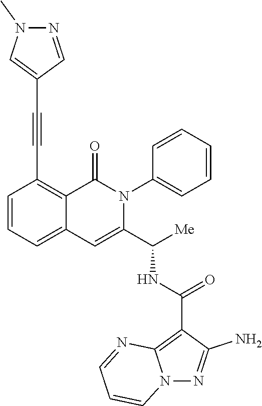 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor.

In one embodiment, the renal cell carcinoma is clear cell renal cell carcinoma. In one embodiment, the renal cell carcinoma (e.g., clear cell renal cell carcinoma) patient is naive to immune therapy (e.g., immune checkpoint therapy, e.g., anti-PD-1 and/or anti-PD-L1 therapy). In one embodiment, the renal cell carcinoma (e.g., clear cell renal cell carcinoma) patient has been previously treated with one or more (e.g., two or more) immune therapy (e.g., immune checkpoint therapy, e.g., anti-PD-1 and/or anti-PD-L1 therapy).

In one embodiment, the renal cell carcinoma is locally advanced and/or metastatic. In one embodiment, the renal cell carcinoma is advanced. In one embodiment, the renal cell carcinoma is locally advanced. In one embodiment, the renal cell carcinoma is metastatic.

In one embodiment, the PD-L1 inhibitor is atezolizumab, YW243.55.S70, MSB0010718C, MDX-1105, or MEDI-4736. In one embodiment, the PD-L1 inhibitor is atezolizumab. In one embodiment, atezolizumab is administered intravenously at about 1200 mg every 3 weeks. In one embodiment, atezolizumab is administered intravenously at a dose of about 1200 mg on day 1 of one or more 21-day cycles.

In one embodiment, the administration of the compound and PD-L1 inhibitor is further in combination with bevacizumab. In one embodiment, n bevacizumab is administered intravenously at a dose of about 15 mg/kg on day 1 of one or more 21-day cycles.

In one embodiment, the method is for treating renal cell carcinoma as front-line treatment. In one embodiment, the method is for treating renal cell carcinoma as first-line treatment.

In one embodiment, the method is for treating (PD-L1 negative) renal cell carcinoma (RCC) with Compound 1, in combination with atezolizumab and bevacizumab.

In one embodiment, provided herein is a method for treating urothelial carcinoma in a subject, comprising: administering to the subject a therapeutically effective amount of a compound of the formula:

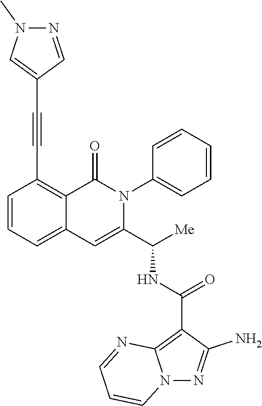 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-1 inhibitor or a PD-L1 inhibitor, wherein the urothelial carcinoma is PD-L1 negative.

In one embodiment, provided herein is a method for treating urothelial carcinoma in a subject, comprising: (i) identifying the urothelial carcinoma in the subject to be PD-L1 negative, and (ii) administering to the subject a therapeutically effective amount of a compound of the formula:

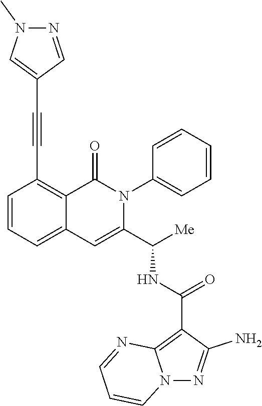 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-1 inhibitor or a PD-L1 inhibitor.

In one embodiment, provided herein is a method for treating urothelial carcinoma in a subject, comprising: administering to the subject a therapeutically effective amount of a compound of the formula:

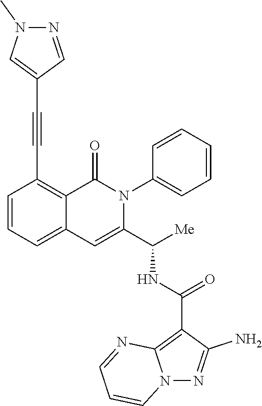 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-1 inhibitor, wherein the urothelial carcinoma is PD-L1 negative.

In one embodiment, provided herein is a method for treating urothelial carcinoma in a subject, comprising: (i) identifying the urothelial carcinoma in the subject to be PD-L1 negative, and (ii) administering to the subject a therapeutically effective amount of a compound of the formula:

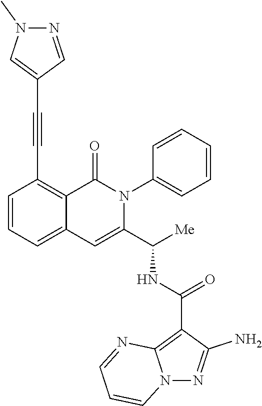 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-1 inhibitor.

In one embodiment, provided herein is a method for treating urothelial carcinoma in a subject, comprising: administering to the subject a therapeutically effective amount of a compound of the formula:

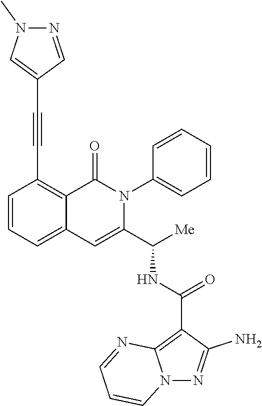 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor, wherein the urothelial carcinoma is PD-L1 negative.

In one embodiment, provided herein is a method for treating urothelial carcinoma in a subject, comprising: (i) identifying the urothelial carcinoma in the subject to be PD-L1 negative, and (ii) administering to the subject a therapeutically effective amount of a compound of the formula:

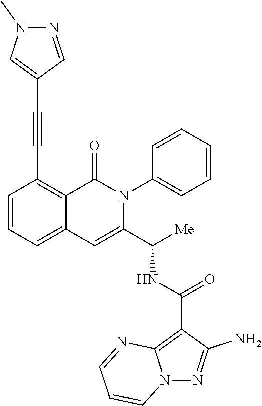 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with a PD-L1 inhibitor.

In one embodiment, the urothelial carcinoma patient is naive to immune therapy (e.g., immune checkpoint therapy, e.g., anti-PD-1 and/or anti-PD-L1 therapy). In one embodiment, the urothelial carcinoma patient has been previously treated with one or more (e.g., two or more) immune therapy (e.g., immune checkpoint therapy, e.g., anti-PD-1 and/or anti-PD-L1 therapy).

In one embodiment, the urothelial carcinoma is locally advanced and/or metastatic. In one embodiment, the urothelial carcinoma is advanced. In one embodiment, the urothelial carcinoma is locally advanced. In one embodiment, the urothelial carcinoma is metastatic.

In one embodiment, the subject is checkpoint-naïve and has progressed or recurred following treatment with platinum-based chemotherapy. In one embodiment, the subject is naïve to immune checkpoint therapy. In one embodiment, the urothelial carcinoma has progressed following treatment with platinum-based chemotherapy. In one embodiment, the urothelial carcinoma has recurred following treatment with platinum-based chemotherapy.

In one embodiment, the patient has progression or refractory disease. In one embodiment, the patient has had at least 1 platinum-based chemotherapy regimen for the treatment of metastatic (Stage IV) or locally advanced unresectable disease. In one embodiment, the patient has disease recurrence within 1 year of completing a platinum-based neoadjuvant or adjuvant therapy. In one embodiment, the patient has been treated or been ineligible for treatment with a fibroblast growth factor receptor (FGFR) inhibitor if the patient has known FGFR3 or FGFR2 genetic alterations.

In one embodiment, the administration of the compound is in combination with a PD-1 inhibitor. In one embodiment, the PD-1 inhibitor is nivolumab, pembrolizumab, pidilizumab, AMP-244, or AMP-514. In one embodiment, the PD-1 inhibitor is nivolumab. In one embodiment, nivolumab is administered intravenously at a dose of about 480 mg once per 4 weeks (Q4W). In one embodiment, the PD-1 inhibitor is pembrolizumab.

In one embodiment, the administration of the compound is in combination with a PD-L1 inhibitor. In one embodiment, the PD-L1 inhibitor is atezolizumab, YW243.55.S70, MSB0010718C, MDX-1105, or MEDI-4736. In one embodiment, the PD-L1 inhibitor is atezolizumab. In one embodiment, atezolizumab is administered intravenously at a dose of about 840 mg on days 1 and 15 of one or more 28-day cycles. In one embodiment, atezolizumab is administered intravenously at a dose of about 1200 mg on day 1 of one or more 21-day cycles.

In one embodiment, the method is for treating platinum-refractory, I/O naïve patients with advanced urothelial cancer, with Compound 1 in combination with nivolumab.

In one embodiment, provided herein is a method for treating a PD-L1 negative patient with immune therapy-naïve, advanced urothelial carcinoma, comprising administering to the patient a therapeutically effective amount of a compound of the formula:

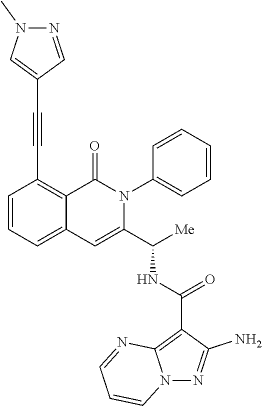 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with nivolumab.

In one embodiment, provided herein is a method for treating a PD-L1 negative patient with immune therapy-naïve, advanced urothelial carcinoma, comprising: (i) identifying the patient to be PD-L1 negative, and (ii) administering to the patient a therapeutically effective amount of a compound of the formula:

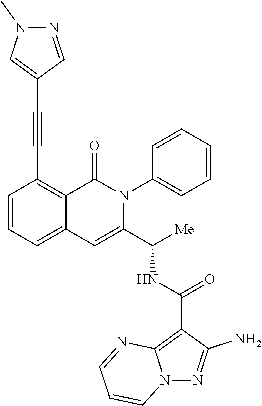 [SMILES: C[C@H](NC(=O)c1c(N)nn2cccnc12)c1cc2cccc(C#Cc3cnn(C)c3)c2c(=O)n1-c1ccccc1]

or a pharmaceutically acceptable salt thereof, in combination with nivolumab.

In one embodiment, the compound is administered orally at a dose of about 30 mg once daily, and nivolumab is administered intravenously at a dose of about 480 mg once per 4 weeks (Q4W). In one embodiment, nivolumab is administered by IV infusion over 30±5 minutes.

In one embodiment, the compound, or a pharmaceutically acceptable salt thereof, is administered orally. In one embodiment, free base of the compound is administered. In one embodiment, a pharmaceutically acceptable salt of the compound is administered.

In one embodiment, the compound, or a pharmaceutically acceptable salt thereof, is administered at a dose of about 10 to about 60 mg once daily. In one embodiment, the compound, or a pharmaceutically acceptable salt thereof, is administered at a dose of about 10, about 15, about 20, about 25, about 30, about 35, about 40, about 45, about 50, about 55, or about 60 mg once daily. In one embodiment, the compound, or a pharmaceutically acceptable salt thereof, is administered at a dose of about 20 mg once daily. In one embodiment, the compound, or a pharmaceutically acceptable salt thereof, is administered at a dose of about 30 mg once daily. In one embodiment, the compound, or a pharmaceutically acceptable salt thereof, is administered at a dose of about 40 mg once daily.

In one embodiment, the administration of the compound results in an observed maximum plasma concentration (Cₘₐₓ) of the compound of no more than 5 μg/mL. In one embodiment, the Cₘₐ of the compound is no more than 4.5 μg/mL. In one embodiment, the Cₘₐₓ of the compound is no more than 4 g/mL. In one embodiment, the Cₘₐₓ of the compound is no more than 3.5 μg/mL. In one embodiment, the Cₘₐₓ of the compound is no more than 3 μg/mL.

In one embodiment, the administration of the compound results in a geometric mean observed maximum plasma concentration (Cₘₐₓ) of the compound of no more than 2 μg/mL. In one embodiment, the geometric mean Cₘₐₓ of the compound is no more than 1.8 μg/mL. In one embodiment, the geometric mean Cₘₐₓ of the compound is no more than 1.6 μg/mL. In one embodiment, the geometric mean Cₘₐₓ of the compound is no more than 1.4 μg/mL. In one embodiment, the geometric mean Cₘₐₓ of the compound is no more than 1.2 μg/mL. In one embodiment, the geometric mean Cₘₐₓ of the compound is no more than 1 μg/mL.

In one embodiment, the administration of the compound results in an area under the concentration time curve (AUC₀₋₂₄) of the compound of no more than 100 μg×hr/mL. In one embodiment, the AUC₀₋₂₄ of the compound is no more than 90 μg×hr/mL. In one embodiment, the AUC₀₋₂₄ of the compound is no more than 80 μg×hr/mL. In one embodiment, the AUC₀₋₂₄ of the compound is no more than 70 g×hr/mL. In one embodiment, the AUC₀₋₂₄ of the compound is no more than 60 μg×hr/mL. In one embodiment, the AUC₀₋₂₄ of the compound is no more than 50 μg×hr/mL.

In one embodiment, the administration of the compound results in a geometric mean area under the concentration time curve (AUC₀₋₂₄) of the compound of no more than 35 μg×hr/mL. In one embodiment, the geometric mean AUC₀₋₂₄ of the compound is no more than 30 μg×hr/mL. In one embodiment, the geometric mean AUC₀₋₂₄ of the compound is no more than 25 μg×hr/mL. In one embodiment, the geometric mean AUC₀₋₂₄ of the compound is no more than 20 μg×hr/mL. In one embodiment, the geometric mean AUC₀₋₂₄ of the compound is no more than 15 μg×hr/mL.

In one embodiment, the compound is administered in 28-day cycles.

In one embodiment, the PK parameters provided herein (e.g., Cₘₐₓ, AUC₀₋₂₄) are measured when the PK profile of the compound reaches a stable status in the patient. In one embodiment, the compound is administered in 28-day cycles and the PK parameters (Cₘₐₓ or AUC₀₋₂₄) are measured around Cycle 2 Day 1.

In one embodiment, the treatment provided herein (e.g., the administration of the compound and/or second agent at specific dose and/or according to specific regimen) results in decreased adverse events. In one embodiment, the adverse event is a hepatic adverse event (e.g., as described in the clinical trial examples provided herein). In one embodiment, the treatment results in Grade 3 or higher hepatic adverse events in no more than 20% of the subjects (patients). In one embodiment, administration of the compound at a dose of 30 mg once daily results in Grade 3 or higher hepatic adverse events in no more than 20% of the subjects (patients).

In one embodiment, a method for treating a cancer that is PD-L1 negative as provided herein means the purpose of the method (e.g., the purpose of administering the compound and/or the additional agents) is to obtain therapeutic benefit on the subject (as compared to other purpose such as serving as a control to treatment on a cancer that is PD-L1 positive). In one embodiment, the treatment results in complete response (CR) in the PD-L1 negative cancer. In one embodiment, the treatment results in partial response (PR) in the PD-L1 negative cancer. In one embodiment, the treatment results in at least partial response (CR+PR) in the PD-L1 negative cancer. In one embodiment, the treatment results in stable disease (SD) in the PD-L1 negative cancer. In one embodiment, the treatment results in at least stable disease (CR+PR+SD) in the PD-L1 negative cancer. In one embodiment, the treatment results in an increase in progression free survival (PFS), overall survival (OS), overall response rate (ORR), complete response (CR), partial response (PR), or duration of response (DOR).

In one embodiment, PFS is, per RECIST v1.1 by ICRR, defined as the time from the date of randomization to the date of documented disease progression or death due to any cause. In one embodiment, PFS is defined as the time from the date of first treatment to the date of documented disease progression or death due to any cause. In one embodiment, OS is defined as the time from the date of randomization to the date of death from any cause. In one embodiment, OS is defined as the time from the date of first treatment to the date of death from any cause. In one embodiment, ORR is with objective response defined as best response of complete response (CR) or partial response (PR), as determined by RECIST v1.1 and iRECIST. In one embodiment, DOR is defined as the time from the first objective response (CR or PR) to documented disease progression in patients with CR or PR.

In one embodiment, provided herein are methods for achieving a complete response, partial response, or stable disease in a PD-L1 negative cancer patient, comprising administering to the patient a therapeutically effective amount of a compound provided herein (e.g., Compound 1), optionally in combination with a second agent provided herein (e.g., immune checkpoint therapy, e.g., anti-PD-1 and/or anti-PD-L1 therapy).

In one embodiment, provided herein are methods for improving progression free survival (PFS), overall survival (OS), overall response rate (ORR), or duration of response (DOR) in a PD-L1 negative cancer patient, comprising administering to the patient a therapeutically effective amount of a compound provided herein (e.g., Compound 1), optionally in combination with a second agent provided herein (e.g., immune checkpoint therapy, e.g., anti-PD-1 and/or anti-PD-L1 therapy).

In one embodiment, the improvement in progression free survival (PFS), overall survival (OS), overall response rate (ORR), or duration of response (DOR) is compared to a reference value accepted in the field. In one embodiment, the improvement is compared to monotherapy by an immune checkpoint therapy, e.g., anti-PD-1 and/or anti-PD-L1 therapy, as approved by FDA or equivalent agency for treating the cancer of interest. In one embodiment, the reference value is established from a clinical trial. For example, the values from CheckMate-275 (a nivolumab monotherapy trial) can be used as the reference values to assess the improvement in a method provided herein.

The statistical analysis of the adverse events provided herein (e.g., percent of hepatic adverse events in the patients) and treatment outcomes provided herein (e.g., PFS, OS, ORR, DOR) could be conducted on individual patient, or patient population (or a representative patient subpopulation), as a skilled artisan in the field deem appropriate.

In one embodiment, the treatment results in increased immune activation. In one embodiment, the increased immune activation comprises increased T cell reinvigoration.

In one embodiment, the treatment results in decreased immune suppression. In one embodiment, the decreased immune suppression comprises decreased MDSC.

In on embodiment, the changes in immune activation or immune suppression are compared to a reference value. In one embodiment, the reference value is measured in the subject before the treatment. In one embodiment, the reference value is measured in a healthy subject.

Patients are often screened for PD-L1 using PD-L1 IHC companion/complementary diagnostic assays, since PD-L1 positive patients often respond to immunotherapy, while PD-L1 negative patients do not. Currently, there are few therapies that have been shown to treat PD-L1 negative patients. Often patients who do not demonstrate PD-L1 positivity through a validated IHC assay are not able to receive treatment (i.e., PD-L1 negative patients are screened out of treatment). In embodiments of the methods provided herein, however, the cancer patients are screened for PD-L1 negativity and PD-L1 negative patients are pre-selected for treatment (i.e., PD-L1 negative patients are screened in for treatment).

In one embodiment, the status of PD-L1 is determined by assays generally known to those skilled in the art, such as those reported in Cheung et al., Appl. Immunohistochem. Mol. Morphol. 2019, 27(10): 699-714; Davis et al., Journal for ImmunoTherapy of Cancer, 2019, 7, Article number: 278; and Kim et al., J Thorac. Oncol. 2018, 13(5):636-648; each of which is incorporated herein by reference in its entirety.

In one embodiment, a cancer provided herein is being determined to be PD-L1 negative or positive on the basis of immunohistochemistry (IHC) results. In one embodiment, the IHC result is based on PD-L1 expression on tumor cells (TCs). In one embodiment, the IHC result is based on PD-L1 expression on tumor-infiltrating immune cells (ICs). In one embodiment, TC0 and IC0 are defined as PD-L1 expression less than 1%, TC1 and IC1 as at least 1% but less than 10%, TC2 and IC2 as 10% or more but less than 50%, and TC3 and IC3 as 50% or more.

In one embodiment, a cancer provided herein (e.g., breast cancer or renal cell carcinoma) is PD-L1 negative based on IHC defined as IC0. In one embodiment, a cancer provided herein (e.g., breast cancer or renal cell carcinoma) is PD-L1 positive based on IHC defined as IC1/2/3.

In one embodiment, PD-L1 expression is measured in baseline/archival tumor biopsies with Dako PD-L1 immunohistochemical 28-8 pharmDx kit approved for nivolumab in urothelial cancer, except 2 biopsies tested with 22C3 PD-L1 antibody prior to study. Tumor Proportion Score≥1% cutoff for PD-L1 (+); Tumor Proportion Score<1% means PD-L1 negative.

### Pharmaceutical Compositions

In some embodiments, provided herein are pharmaceutical compositions comprising a compound as provided herein (e.g., Compound 1), or an enantiomer, a mixture of enantiomers, or a mixture of two or more diastereomers thereof, or a pharmaceutically acceptable form thereof (e.g., pharmaceutically acceptable salts, hydrates, solvates, isomers, prodrugs, and isotopically labeled derivatives), and a pharmaceutically acceptable excipient, diluent, or carrier, including inert solid diluents and fillers, sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

In some embodiments, provided herein are pharmaceutical compositions comprising a compound provided herein (e.g., Compound 1), or a salt, or solvate, or solvate of a salt thereof, or a mixture thereof, and a bulking agent (filler or carrier), and optionally a disintegrant and a lubricant. In some embodiments, provided herein are pharmaceutical compositions comprising a compound provided herein (e.g., Compound 1), or a salt, or solvate, or solvate of a salt thereof, or a mixture thereof, and a pharmaceutically acceptable excipient, diluent, or carrier, including inert solid diluents and fillers, sterile aqueous solution and various organic solvents, permeation enhancers, solubilizers and adjuvants. In one embodiment, provided herein is a pharmaceutical composition comprising a compound provided herein (e.g., Compound 1) and a pharmaceutical acceptable excipient thereof. In one embodiment, provided herein is a pharmaceutical composition consisting essentially of a compound provided herein (e.g., Compound 1). In one embodiment, the compound is present in said composition in an amount of at least about 80% by weight. In one embodiment, the compound is present in said composition in an amount of at least about 90% by weight.

In one embodiment, the compound in the pharmaceutical composition is an amorphous form of Compound 1. In one embodiment, the amorphous form of Compound 1 is prepared by dissolving a crystalline form (e.g., Form 1) of Compound 1 in one or more solvents to form a solution; and removing the solvent of the solution to provide the amorphous form of Compound 1. In one embodiment, the solvent is removed by spray drying.

In one embodiment, the pharmaceutical composition comprises one or more excipients selected from bulking agents (or fillers), disintegrants, lubricants, and capsule shell. In one embodiment, the bulking agent is mannitol or pre-gelatinized starch. In another embodiment, the disintegrant is croscarmellose sodium. In another embodiment, the lubricant is magnesium stearate. In one embodiment, the capsule shell is HPMC capsule shell. In one embodiment, the pharmaceutical composition comprises one or more excipients selected from mannitol, pre-gelatinized starch, croscarmellose sodium, magnesium stearate, and HPMC capsule shell.

In one embodiment, the amount of Compound 1 in the pharmaceutical composition is about 1 mg to about 100 mg, about 1 mg to about 75 mg, about 1 mg to about 50 mg, about 1 mg to about 40 mg, about 5 mg to about 50 mg, about 5 mg to about 30 mg, about 5 mg to about 10 mg, about 5 mg, or about 30 mg. In one embodiment, the amount is about 5 mg, 10 mg, 15 mg, 20 mg, 30 mg, 40 mg, 50 mg, 75 mg, or 100 mg. In one embodiment, the amount is about 5 mg or 30 mg. In one embodiment, the amount of Compound 1 in the pharmaceutical composition is about 1.5% to about 25% w/w, about 1.5% to about 15% w/w, about 1.5% to about 10% w/w, about 1% to about 25% w/w, about 1% to about 15% w/w, or about 1% to about 10% w/w. In one embodiment, the amount of Compound 1 in the pharmaceutical composition is about 1% to about 10% w/w. In one embodiment, the amount of Compound 1 in the pharmaceutical composition is about 1, 1.5, 2, 2.5, 3, 3.5, 4, 4.5, 5, 5.5, 6, 6.5, 7, 7.5, 8, 8.5, 9, 9.5, or 10% w/w. In one embodiment, the amount of Compound 1 in the pharmaceutical composition is about 1.9%, or about 9.4%. In one embodiment, the amount of Compound 1 is about 1.92% or about 9.38%.

In one embodiment, the bulking agent (or filler) (e.g., starch and mannitol) in a pharmaceutical composition is about 80% to about 95% w/w, about 85% to about 95% w/w, or about 90% to about 95% w/w. In one embodiment, the bulking agent (or filler) (e.g., starch and mannitol) in a pharmaceutical composition is about 80%, about 85%, about 90%, or about 95% w/w. In one embodiment, the bulking agent (or filler) (e.g., starch and mannitol) in a pharmaceutical composition is about 93% w/w, about 86% w/w, about 92.3% w/w, or about 85.1% w/w. In one embodiment, the bulking agent is about 93% w/w. In one embodiment, the bulking agent is about 85% w/w. In one embodiment, the bulking agent is starch, mannitol, or a mixture thereof. In one embodiment, the bulking agent is a mixture of starch and mannitol. In one embodiment, the weight ratio of starch to mannitol is from about 1:3 to about 3:1. In one embodiment, the bulking agent is an about 1:1 mixture of starch and mannitol. In one embodiment, the starch is pre-gelatinized starch.

In one embodiment, the disintegrant (e.g., croscarmellose sodium) in a pharmaceutical composition is about 1% to about 20% w/w, about 1% to about 15% w/w, about 1% to about 10% w/w, about 2.5% to about 7.5% w/w, about 1% to about 50% w/w, or about 50% w/w. In one embodiment, the disintegrant is about 1%, about 2%, about 3%, about 4%, about 5%, about 6%, about 7%, about 8%, about 9%, or about 10% w/w. In one embodiment, the disintegrant is about 5% w/w.

In one embodiment, the lubricant (e.g., magnesium stearate) in a pharmaceutical composition is about 0.1% to about 10% w/w, about 0.1% to about 5% w/w, or about 0.1% to about 1% w/w. In one embodiment, the lubricant is about 0.1%, about 0.2%, about 0.3%, about 0.4%, about 0.5%, about 0.6%, about 0.7%, about 0.8%, about 0.9%, or about 1% w/w. In one embodiment, the lubricant is about 0.5% w/w.

In one embodiment, provided herein is a pharmaceutical composition comprising an amorphous form of Compound 1, or a salt, or solvate, or solvate of a salt thereof, or a mixture thereof, and a bulking agent (filler or carrier), and optionally a disintegrant and a lubricant. In one embodiment, provided herein is a pharmaceutical composition comprising about 1% to about 10% w/w of an amorphous form of Compound 1, or a salt, or solvate, or solvate of a salt thereof, or a mixture thereof, about 80% to about 95% w/w of a bulking agent, about 2.5% to about 7.5% w/w of a disintegrant, and about 0.10% to about 1% w/w of a lubricant.

In one embodiment, provided herein is a pharmaceutical composition comprising about 5 to 30 mg Compound 1 (e.g., amorphous), pre-gelatinized starch, and mannitol. In one embodiment, the pharmaceutical composition further comprises croscarmellose sodium and magnesium stearate.

In one embodiment, the pharmaceutical composition is formulated as follows: about 5 mg of Compound 1 (e.g., amorphous), about 120 mg of pre-gelatinized starch, about 120 mg of mannitol, about 13 mg of croscarmellose sodium, and about 1.3 mg of magnesium stearate. In embodiment, the pharmaceutical composition is formulated as a capsule. In one embodiment, the pharmaceutical composition is formulated as follows: about 5 mg of Compound 1 (e.g., amorphous), about 120.35 mg of pre-gelatinized starch, about 120.35 mg of mannitol, about 13.00 mg of croscarmellose sodium, and about 1.3 mg of magnesium stearate. In embodiment, the pharmaceutical composition is formulated as a capsule.

In one embodiment, the pharmaceutical composition is formulated as follows: about 30 mg of Compound 1 (e.g., amorphous), about 136 mg of pre-gelatinized starch, about 136 mg of mannitol, about 16 mg of croscarmellose sodium, and about 1.6 mg of magnesium stearate. In one embodiment, the pharmaceutical composition is formulated as follows: about 30 mg of Compound 1 (e.g., amorphous), about 136.20 mg of pre-gelatinized starch, about 136.20 mg of mannitol, about 16.00 mg of croscarmellose sodium, and about 1.60 mg of magnesium stearate. In embodiment, the pharmaceutical composition formulated as a capsule.

In some embodiments, a pharmaceutical composition described herein includes a second active agent such as an additional therapeutic agent, (e.g., a chemotherapeutic agent).

In some embodiments, provided herein is a pharmaceutical composition for oral administration (e.g., capsule) comprising: (a) about 5 mg of amorphous Compound 1; (b) about 120.35 mg of pre-gelatinized starch; (c) about 120.35 mg of mannitol; (d) about 13 mg of croscarmellose sodium; and (e) about 1.3 mg of magnesium stearate.

In some embodiments, provided herein is a pharmaceutical composition for oral administration (e.g., capsule) comprising: (a) about 30 mg of amorphous Compound 1; (b) about 136.2 mg of pre-gelatinized starch; (c) about 136.2 mg of mannitol; (d) about 16 mg of croscarmellose sodium; and (e) about 1.6 mg of magnesium stearate.

In one embodiment, the pharmaceutical composition is an oral dosage form. In one embodiment, the oral dosage form is a capsule. In another embodiment, the oral dosage form is a tablet. In one embodiment, the capsule shell is Swedish orange or white.

Additional polymorphic forms, pharmaceutical composition, and formulations for Compound 1 are described in WO 2017/048702; the entirety of which is incorporated herein by reference.

### Dosage

A compound described herein (e.g., a PI3K-γ inhibitor such as Compound 1) can be delivered in the form of pharmaceutically acceptable compositions which comprise a therapeutically effective amount of one or more compounds described herein and/or one or more additional therapeutic agents such as a chemotherapeutic, formulated together with one or more pharmaceutically acceptable excipients. In some instances, the compound described herein and the additional therapeutic agent are administered in separate pharmaceutical compositions and can (e.g., because of different physical and/or chemical characteristics) be administered by different routes (e.g., one therapeutic is administered orally, while the other is administered intravenously). In other instances, the compound described herein and the additional therapeutic agent can be administered separately, but via the same route (e.g., both orally or both intravenously). In still other instances, the compound described herein and the additional therapeutic agent can be administered in the same pharmaceutical composition.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a compound described herein and/or a chemotherapeutic will be that amount of the compound which, in some embodiments, can be the lowest dose effective to produce a therapeutic effect. Such an effective dose will generally depend upon the factors described herein. Generally, doses of the compounds described herein for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to 1000 mg, or about 0.01 mg to about 500 mg per day, or about 0.1 mg to about 500 mg per day, or about 1 mg to 50 mg per day, or about 5 mg to 40 mg per day. In some embodiments, range is from about 1 mg to about 100 mg, about 1 mg to about 200 mg, about 1 mg to about 500 mg, about 1 mg to about 1000 mg, about 100 mg to about 200 mg, about 100 mg to about 500 mg, about 100 to about 750 mg, about 100 mg to about 1000 mg. An exemplary dosage is about 10 to 30 mg per day. In some embodiments, for a 70 kg human, a suitable dose would be about 0.05 to about 7 g/day, such as about 0.05 to about 2.5 g/day. Actual dosage levels of the active ingredients in the pharmaceutical compositions described herein can be varied so as to obtain an amount of the active ingredient which is effective to achieve the desired therapeutic response for a particular patient, composition, and mode of administration, without being toxic to the patient. In some instances, dosage levels below the lower limit of the aforesaid range can be more than adequate, while in other cases still larger doses can be employed without causing any harmful side effect, e.g., by dividing such larger doses into several small doses for administration throughout the day.

Pharmacokinetic studies in animals provided herein suggest efficacious dose ranges for Compound 1. Generally speaking, to specifically inhibit PI3K-γ, one can administer a dose of a specific PI3K-γ inhibitor that results in an unbound plasma concentration of the drug that is above a predetermined threshold (e.g., the IC50, IC60, IC70, IC80, or IC90 for PI3K-γ) for a selected time (e.g., 1 hour, 2 h, 3 h, 6 h, 12 h, 24 h, 2 days, 3 d, 5 d, or 7 d). This dose may be selected such that the plasma concentration is below a second predetermined threshold (e.g., the IC20, IC30, IC40, or IC50 for PI3K-δ, -α, or β) the for a selected time (e.g., 1 hour, 2 h, 3 h, 6 h, 12 h, 24 h, 2 days, 3 d, 5 d, or 7 d). In some embodiments, the PI3K-γ inhibitor, e.g., Compound 1, is administered at a dose that results in an unbound plasma concentration of Compound 1 that is above the IC90 of PI3K-γ for at least 1 hour, 2 h, 3 h, 6 h, 12 h, or 24 h. In some embodiments, the PI3K-γ inhibitor, e.g., Compound 1, is administered at a dose that results in an unbound plasma concentration of Compound 1 that is above the IC50 of PI3K-γ for at least 1 hour, 2 h, 3 h, 6 h, 12 h, or 24 h.

Based on non-human animal studies, a predicted human dose to achieve exposure at the IC90 for PI3K-γ is approximately 2 mg. Accordingly, in some embodiments, the methods herein involve administering a selective PI3K-γ inhibitor, e.g., Compound 1, to a human, wherein each dose is about 2 mg, 1-3 mg, 1-5 mg, 1-10 mg, 0.5-20 mg, or 0.1-50 mg. In some embodiments, the dose (e.g., a therapeutically effective dose) is about 2 mg, 1-3 mg, 1-5 mg, 1-10 mg, 0.5-20 mg, 0.1-50 mg, 0.1-75 mg, 0.5-75 mg, 1-75 mg, 0.1-100 mg, 0.5-100 mg, or 1-100 mg. In some embodiments, the dose is about 1-10 mg. In some embodiments, the dose is about 1-50 mg. In some embodiments, the dose is about 1-100 mg. In a 70 kg human, a 2 mg dose corresponds to 0.029 mg/kg. Accordingly, in some embodiments, the methods herein involve administering a selective PI3K-γ inhibitor, e.g., Compound 1, to a human, wherein each dose is about 0.029 mg/kg, 0.014-0.14 mg/kg, 0.02-0.04 mg/kg, 0.01-0.05 mg/kg, 0.01-0.1, or 0.01-0.5 mg/kg.

In one embodiment, the therapeutically effective dose of the compound is about 2 mg, about 1-3 mg, about 1-5 mg, about 1-10 mg, about 0.5-20 mg, about 0.1-50 mg per day, about 0.1-75 mg per day, about 0.1-100 mg per day, about 0.1-250 mg per day, about 0.1-500 mg per day, about 0.1-1000 mg per day, about 1-50 mg per day, about 1-75 mg per day, about 1-100 mg per day, about 1-250 mg per day, about 1-500 mg per day, about 1-1000 mg per day, about 10-50 mg per day, about 10-75 mg per day, about 10-100 mg per day, about 10-250 mg per day, about 10-500 mg per day, about 10-1000 mg per day, about 100-500 mg per day, or about 100-1000 mg per day. In one embodiment, the therapeutically effective dose is about 0.029 mg/kg, about 0.014-0.14 mg/kg, about 0.02-0.04 mg/kg, about 0.01-0.05 mg/kg, about 0.01-0.1, or about 0.01-0.5 mg/kg. In one embodiment, the compound is administered once every two days. In one embodiment, wherein the compound is administered once per day. In one embodiment, the compound is administered twice per day.

In one embodiment, the compound is administered at a dose such that the level of the compound in the subject is higher than the compound's IC50 of PI3K-gamma inhibition during at least 70%, 80%, 90%, 95%, 97%, 98%, or 99% of a selected time period, e.g., 6 hours, 12 hours, 24 hours, or 48 hours immediately following the administration. In one embodiment, the compound is administered at a dose such that the level of the compound in the subject is higher than the compound's IC90 of PI3K-gamma inhibition during at least 50%, 60%, 70%, 80%, 90%, 95%, 97%, 98%, or 99% of a selected time period, e.g., 6 hours, 12 hours, 24 hours, or 48 hours, immediately following the administration. In one embodiment, the compound is administered at a dose such that the level of the compound in the subject does not rise higher than the compound's IC20 or IC50 of PI3K-delta inhibition within a selected time period, e.g., 6 hours, 12 hours, 24 hours, or 48 hours, immediately following the administration. In one embodiment, the level of the compound is measured from the subject's plasma. In one embodiment, the level of the compound is measured from the subject's tissue. In one embodiment, the compound is administered at a dose such that it provides at least 50% inhibition of PI3K-gamma in the subject but less than 10% or 20% inhibition of PI3K-delta in the subject.

In some embodiments, the compounds can be administered daily, every other day, three times a week, twice a week, weekly, or bi-weekly. The dosing schedule can include a “drug holiday,” e.g., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

In some embodiments, a compound as provided herein is administered in multiple doses. Dosing can be about once, twice, three times, four times, five times, six times, or more than six times per day. Dosing can be about once a month, about once every two weeks, about once a week, or about once every other day. In another embodiment, a compound as disclosed herein and another agent are administered together from about once per day to about 6 times per day. In another embodiment, the administration of a compound as provided herein and an agent continues for less than about 7 days. In yet another embodiment, the administration continues for more than about 6 days, about 10 days, about 14 days, about 28 days, about two months, about six months, or about one year. In some cases, continuous dosing is achieved and maintained as long as necessary.

Based on non-human animal studies provided herein the oral half-life of Compound 1 in humans is expected to be about 10-13 hours. This finding informs the timing of administration of a PI3K-γ inhibitor such as Compound 1. For instance, in some embodiments, the timing is selected such that an unbound plasma concentration of the drug that is above a predetermined threshold (e.g., the IC50, IC60, IC70, IC80, or IC90 for PI3K-γ) for a selected time (e.g., 1 hour, 2 h, 3 h, 6 h, 12 h, 24 h, 2 days, 3 d, 5 d, or 7 d). The timing of administration may also be chosen such that the plasma level is below a second predetermined threshold (e.g., the IC20, IC30, IC40, or IC50 for PI3K-δ, -α, or β) the for a selected time (e.g., 1 hour, 2 h, 3 h, 6 h, 12 h, 24 h, 2 days, 3 d, 5 d, or 7 d). In some embodiments, the PI3K-γ inhibitor, e.g., Compound 1, is administered with timing that results in an unbound plasma concentration of Compound 1 that is above the IC90 of PI3K-γ for at least 1 hour, 2 h, 3 h, 6 h, 12 h, or 24 h. In some embodiments, the PI3K-γ inhibitor, e.g., Compound 1, is administered with timing that results in an unbound plasma concentration of Compound 1 that is above the IC50 of PI3K-γ for at least 1 hour, 2 h, 3 h, 6 h, 12 h, or 24 h.

Accordingly, in some embodiments, the methods herein involve administering a selective PI3K-γ inhibitor, e.g., Compound 1, to a human, about once per day. In embodiments, the selective PI3K-γ inhibitor, e.g., Compound 1, is administered to a human once every two days. In embodiments, the selective PI3K-γ inhibitor, e.g., Compound 1, is administered to a human twice or three times per day.

Administration of the pharmaceutical compositions as disclosed herein can continue as long as necessary. In some embodiments, an agent as disclosed herein is administered for more than about 1, about 2, about 3, about 4, about 5, about 6, about 7, about 14, or about 28 days. In some embodiments, an agent as disclosed herein is administered for less than about 28, about 14, about 7, about 6, about 5, about 4, about 3, about 2, or about 1 day. In some embodiments, an agent as disclosed herein is administered chronically on an ongoing basis, e.g., for the treatment of chronic effects.

Since the compounds described herein can be administered in combination with other treatments (such as additional chemotherapeutics, radiation or surgery), the doses of each agent or therapy can be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about ito about 50 mg per kilogram of body weight per day. In some embodiments, the dose is about 1 mg/kg, about 5 mg/kg, about 7.5 mg/kg, about 10 mg/kg, about 15 mg/kg, about 20 mg/kg, about 25 mg/kg, about 50 mg/kg, about 75 mg/kg, or about 100 mg/kg per day. In some embodiments, the dose is about 1 mg/kg, about 7.5 mg/kg, about 20 mg/kg, or about 50 mg/kg per day.

In one embodiment, the compound, or a pharmaceutically acceptable salt thereof, is administered to a subject at a dose (e.g., a therapeutically effective dose) of about 2 mg, 1-3 mg, 1-5 mg, 1-10 mg, 0.5-20 mg, or 0.1-50 mg. In one embodiment, the dose (e.g., a therapeutically effective dose) is about 2 mg, 1-3 mg, 1-5 mg, 1-10 mg, 0.5-20 mg, 0.1-50 mg, 0.1-75 mg, 0.5-75 mg, 1-75 mg, 0.1-100 mg, 0.5-100 mg, or 1-100 mg. In one embodiment, the dose is about 1-10 mg. In one embodiment, the dose is about 1-50 mg. In one embodiment, the dose is about 1-100 mg.

In one embodiment, the compound, or a pharmaceutically acceptable salt thereof, is administered to a subject at a dose (e.g., a therapeutically effective dose) of about 0.029 mg/kg, 0.014-0.14 mg/kg, 0.02-0.04 mg/kg, 0.01-0.05 mg/kg, 0.01-0.1, or 0.01-0.5 mg/kg.

In one embodiment, the compound, or a pharmaceutically acceptable salt thereof, is administered to a subject at a treatment schedule chosen from, e.g., once every two days, once per day, or twice per day.

In one embodiment, the compound, or a pharmaceutically acceptable salt thereof, is administered at a dose such that it selectively inhibits PI3K-gamma but achieves less than 10% or 20% inhibition of PI3K-delta.

In one embodiment, the compound, or a pharmaceutically acceptable salt thereof, is administered at a dose such that the compound's level in the subject's blood does not rise higher than a predetermined level, e.g., the IC₅₀ of PI3K-delta, within a selected time period, e.g., 24 hours. In some embodiments of the methods or uses disclosed herein, the PI3K gamma inhibitor or compound is administered at a dose such that the compound's level in the subject's blood does not rise higher than a predetermined level, e.g., the IC₂₀ of PI3K-delta, within a selected time period, e.g., 24 hours.

In one embodiment, the compound, or a pharmaceutically acceptable salt thereof, is administered to a subject in an amount such that the level of the compound in the subject's body is above the IC50 of PI3K-gamma during at least 70%, 80%, 90%, 95%, 97%, 98%, 99%, or 99% of selected time period, e.g., 24 hours, immediately following the administration.

In one embodiment, the compound, or a pharmaceutically acceptable salt thereof, is administered to a subject in an amount such that the level of the compound in the subject's body is above the IC90 of PI3K-gamma during at least 50%, 60%, 70%, 80%, 90%, 95%, 97%, 98%, 99%, or 99% of a selected time period, e.g., 24 hours, immediately following the administration.

In certain embodiments, a therapeutically or prophylactically effective amount of a compound provided herein (e.g., Compound 1), or an enantiomer, a mixture of enantiomers, or a mixture of two or more diastereomers thereof, or a pharmaceutically acceptable form thereof, is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 2 to about 25 mg per day, or from about 5 to about 10 mg per day.

In certain embodiments, the therapeutically or prophylactically effective amount is about 0.1, about 0.2, about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of a compound provided herein (e.g., Compound 1), or an enantiomer, a mixture of enantiomers, or a mixture of two or more diastereomers thereof, or a pharmaceutically acceptable form thereof, for the conditions described herein lie within the range of from about 0.5 mg to about 50 mg per day, in a single once-a-day dose or in divided doses throughout a day. In some embodiments, the dosage ranges from about 1 mg to about 50 mg per day. In some embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage can be 0.5, 1, 2, 3, 4, 5, 10, 15, 20, 25 or 50 mg per day. In another embodiment, the recommended starting dosage can be 0.5, 1, 2, 3, 4, or 5 mg per day. The dose can be escalated to 15, 20, 25, 30, 35, 40, 45 and 50 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

In certain embodiments, the therapeutically or prophylactically effective amount is from about 0.01 to about 25 mg/kg/day, from about 0.01 to about 20 mg/kg/day, from about 0.01 to about 15 mg/kg/day, from about 0.01 to about 10 mg/kg/day, from about 0.05 to about 25 mg/kg/day, from about 0.05 to about 20 mg/kg/day, from about 0.05 to about 15 mg/kg/day, or from about 0.05 to about 10 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

A compound provided herein (e.g., Compound 1), or an enantiomer, a mixture of enantiomers, or a mixture of two or more diastereomers thereof, or a pharmaceutically acceptable form thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as Compound 1, is administered once or more than once each day, for example, for a period of time. The term “continuous” is intended to mean that a therapeutic compound, such as Compound 1, is administered daily for an uninterrupted period of at least 10 days to 52 weeks. The term “intermittent” or “intermittently” as used herein is intended to mean stopping and starting at either regular or irregular intervals. For example, intermittent administration of Compound 1 is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as Compound 1, is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein is administered once a day. In another embodiment, the compound provided herein is administered twice a day. In yet another embodiment, the compound provided herein is administered three times a day. In still another embodiment, the compound provided herein is administered four times a day.

In one embodiment, a compound provided herein (e.g., Compound 1), or an enantiomer, a mixture of enantiomers, or a mixture of two or more diastereomers thereof, or a pharmaceutically acceptable form thereof, is administered twice per day (BID). In one embodiment, the dose is about 0.1, 0.2, 0.25, 0.5, 1, 2, 2.5, 5, 10, 15, 20, 25, 30, 35, 40, 45, or 50 mg BID.

In one embodiment, a compound provided herein (e.g., Compound 1), or an enantiomer, a mixture of enantiomers, or a mixture of two or more diastereomers thereof, or a pharmaceutically acceptable form thereof, is administered once daily (QD). In one embodiment, the dose is about 0.1, 0.2, 0.25, 0.5, 1, 2, 2.5, 5, 10, 15, 20, 25, 30, 35, 40, 45, or 50 mg QD.

In one embodiment, the amount administered is sufficient to provide an area under the curve (AUC) of the compound, ranging from about 50 to about 10,000 ng*hr/mL, about 100 to about 50,000 ng*hr/mL, from about 100 to 25,000 ng*hr/mL, or from about 10,000 to 25,000 ng*hr/mL. In certain embodiments, a compound provided herein (e.g., Compound 1), or an enantiomer, a mixture of enantiomers, or a mixture of two or more diastereomers thereof, or a pharmaceutically acceptable form thereof, is administered once per day from one day to six months, from one week to three months, from one week to four weeks, from one week to three weeks, or from one week to two weeks.

In certain embodiments, the compound provided herein is administered once per day for one week, two weeks, three weeks, or four weeks. In one embodiment, the compound provided herein is administered once per day for one week. In another embodiment, the compound provided herein is administered once per day for two weeks. In yet another embodiment, the compound provided herein is administered once per day for three weeks. In still another embodiment, the compound provided herein is administered once per day for four weeks.

In certain embodiments, a compound provided herein (e.g., Compound 1), or an enantiomer, a mixture of enantiomers, or a mixture of two or more diastereomers thereof, or a pharmaceutically acceptable form thereof, is administered twice per day from one day to six months, from one week to three months, from one week to four weeks, from one week to three weeks, or from one week to two weeks. In certain embodiments, the compound provided herein is administered twice per day for one week, two weeks, three weeks, or four weeks. In one embodiment, the compound provided herein is administered twice per day for one week. In another embodiment, the compound provided herein is administered twice per day for two weeks. In yet another embodiment, the compound provided herein is administered twice per day for three weeks. In still another embodiment, the compound provided herein is administered twice per day for four weeks.

The compound provided herein (e.g., Compound 1), or an enantiomer, a mixture of enantiomers, or a mixture of two or more diastereomers thereof, or a pharmaceutically acceptable form thereof, can be delivered as a single dose such as, e.g., a single bolus injection, or oral tablets or pills; or over time, such as, e.g., continuous infusion overtime or divided bolus doses overtime. The compound can be administered repeatedly if necessary, for example, until the patient experiences stable disease or regression, or until the patient experiences disease progression or unacceptable toxicity.

In some embodiments of the methods or uses disclosed herein, the subject is a human and the compound has a half life of about 8-15 hours, or about 10-13 hours.

When a compound provided herein, is administered in a pharmaceutical composition that comprises one or more agents, and the agent has a shorter half-life than the compound provided herein unit dose forms of the agent and the compound provided herein can be adjusted accordingly.

### Kits

In some embodiments, provided herein are kits. The kits can include a compound or pharmaceutical composition as described herein, in suitable packaging, and written material that can include instructions for use, discussion of clinical studies, listing of side effects, and the like. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials.

In some embodiments, a memory aid is provided with the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day.

The kit can further contain another agent. In some embodiments, the compound as disclosed herein and the agent are provided as separate pharmaceutical compositions in separate containers within the kit. In some embodiments, the compound as disclosed herein and the agent are provided as a single pharmaceutical composition within a container in the kit. Suitable packaging and additional articles for use (e.g., measuring cup for liquid preparations, foil wrapping to minimize exposure to air, and the like) are known in the art and can be included in the kit. In other embodiments, kits can further comprise devices that are used to administer the active agents. Examples of such devices include, but are not limited to, syringes, drip bags, patches, and inhalers. Kits described herein can be provided, marketed and/or promoted to health providers, including physicians, nurses, pharmacists, formulary officials, and the like. Kits can also, in some embodiments, be marketed directly to the consumer.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

Kits can further comprise pharmaceutically acceptable vehicles that can be used to administer one or more active agents. For example, if an active agent is provided in a solid form that must be reconstituted for parenteral administration, the kit can comprise a sealed container of a suitable vehicle in which the active agent can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The present disclosure further encompasses anhydrous pharmaceutical compositions and dosage forms comprising an active ingredient, since water can facilitate the degradation of some compounds. For example, water can be added (e.g., about 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. For example, pharmaceutical compositions and dosage forms which contain lactose can be made anhydrous if substantial contact with moisture and/or humidity during manufacturing, packaging, and/or storage is expected. An anhydrous pharmaceutical composition can be prepared and stored such that its anhydrous nature is maintained. Accordingly, anhydrous pharmaceutical compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

The examples and preparations provided below further illustrate and exemplify the compounds as provided herein and methods of preparing such compounds. It is to be understood that the scope of the present disclosure is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers can be obtained by methods known to those skilled in the art.

### EXAMPLES

Biological activities of the compounds (e.g., Compound 1) have been described in WO 2015/051244; additional data of the compounds (e.g., Compound 1) against various cancer cell lines and cancer animal models has been described in WO 2015/143012, each of which is incorporated herein by reference in its entirety.

### Example 1: Phase II Study Safety Run-In Evaluating a Novel Triplet Combination of Compound 1, Atezolizumab (Atezo), and Nab-Paclitaxel (Nab-Pac) as First-Line (1L) Therapy for Locally Advanced or Metastatic Triple-Negative Breast Cancer (TNBC)

Purpose: The IMpassion130 randomized trial in advanced TNBC has demonstrated improved efficacy with the addition of atezo to 1L nab-pac in patients with PD-L1+ tumors. Compound 1 is an oral agent targeting tumor-associated myeloid cells through selective inhibition of PI3K-gamma, with the goal of improving the immune response to the approved doublet combination of atezo and nab-pac. Some results from a completed TNBC safety run-in cohort of a multicenter phase II study are reported below.

Methods: Eligible patients had measurable unresectable locally advanced or metastatic TNBC, ECOG performance status 0/1, and no prior systemic therapy for advanced disease. A safety run-in was completed to assess the safety of the triplet of oral Compound 1 30 mg daily in combination with nab-pac 100 mg/m2 given on days 1, 8, & 15, and IV atezo 840 mg given on days 1 & 15. After establishing tolerability in the safety run-in (n=6), the expansion phase of the phase II study was initiated to enroll a total of approximately 60 patients (30 PD-L1+ and 30 PD-L1−). Cycles are repeated every 28 days until loss of clinical benefit, unacceptable toxicity, or consent withdrawal. The primary efficacy endpoint is confirmed Complete Response (CR) rate per RECIST v1.1. Secondary endpoints include the overall response rate (ORR) and safety assessment. Tumors are assessed every 8 weeks by CT/MRI scan.

Results: Described below are preliminary efficacy data and safety data for the completed safety run-in cohort with 6 patients evaluable for safety and 4 evaluable for response defined as having had at least one post-baseline tumor assessment. 1 CR (1/4) and 3 PRs (3/4) were observed with an ORR of 100% (4/4). Responses were seen irrespective of PD-L1 status. The most common all-grade adverse events were decreased white cell count (66.7%), fatigue (50%), diarrhea (33.3%), hyperglycaemia (33.3%), transaminase elevation (16.7%), pyrexia (16.7%), and rash (16.7%). Most common grade≥3 adverse events occurred in 3 patients (50%) including decreased lymphocytes or neutropenia (33.3%), transaminase elevation (16.7%), fatigue (16.7%), rash (16.7%), and febrile neutropenia (16.7%). Treatment was generally tolerable. Compound 1 30 mg daily was chosen as the dose for combination with nab-pac and atezo for the expansion phase of the study.

Conclusions: The triplet regimen of Compound 1, atezo, and nab-pac shows promising antitumor activity (4 responses/4 evaluable patients), irrespective of PD-L1 biomarker status, and has manageable toxicity.

### Example 2: Phase II Study Initial Data Evaluating a Triplet Combination of Eganelisib (Compound 1), Atezolizumab (Atezo), and Nab-Paclitaxel (Nab-Pac) as First-Line (1L) Therapy for Locally Advanced or Metastatic Triple-Negative Breast Cancer (TNBC)

Background: The addition of atezolizumab (atezo) to nab-paclitaxel (nab-pac) in the IMpassion130 study demonstrated improved efficacy over nab-pac in unresectable locally advanced or metastatic (1L) triple-negative breast cancer (TNBC) patients with PD-L1(+) tumors. Atezo and nab-pac combination received accelerated approval in the US in PD-L1(+), but not in the PD-L1(−), patient population (IMpassion130 Study: ORR in ITT=56%; ORR in PD-L1(+)=59%). Eganelisib (Compound 1) is a selective PI3Kγ inhibitor that reprograms pro-tumor macrophages to relieve immune suppression and activate anti-tumor T cells. The phase II study is designed to evaluate the triple combination therapy of eganelisib, atezo and nab-pac for the treatment of 1L TNBC.

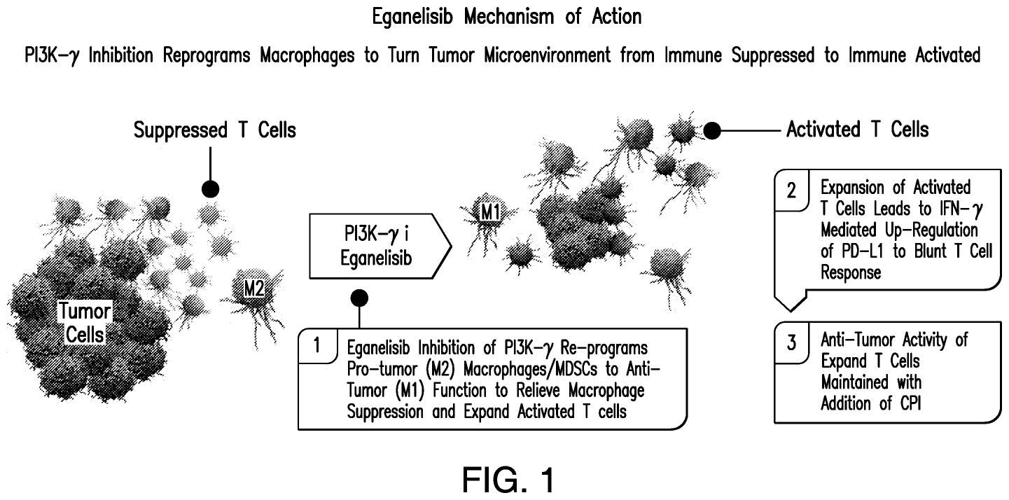

FIG. 1 shows eganelisib (Compound 1) mechanism of action. Eganelisib inhibition of PI3K-γ re-programs pro-tumor (M2) macrophages/MDSCs to anti-tumor (M1) function to relieve macrophage suppression and expand activated T cells. Expansion of activated T cells leads to IFN-γ mediated up-regulation of PD-L1 to blunt T cell response. Anti-tumor activity of expanded T cells maintained with addition of CPI.

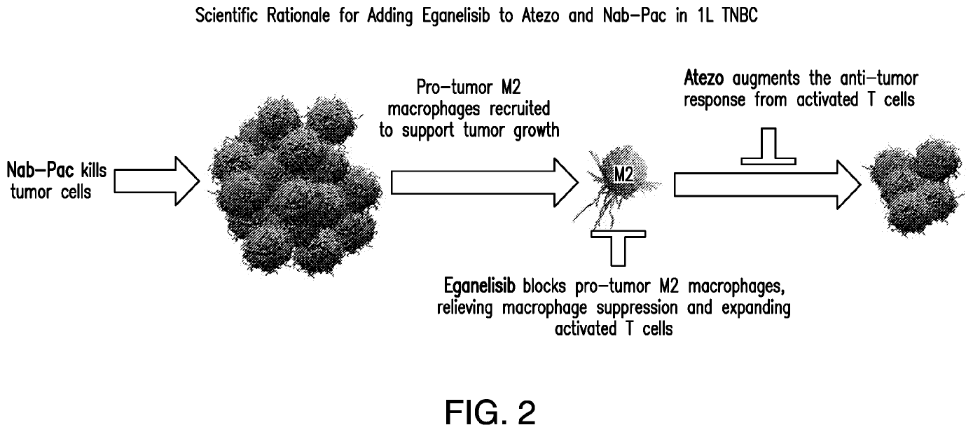

FIG. 2 shows scientific rationale for adding eganelisib to atezo and nab-Pac in 1L TNBC. Nab-Pac kills tumor cells. Pro-tumor M2 macrophages recruited to support tumor growth. Eganelisib blocks pro-tumor M2 macrophages, relieving macrophage suppression and expanding activated T cells. Atezo augments the anti-tumor response from activated T cells.

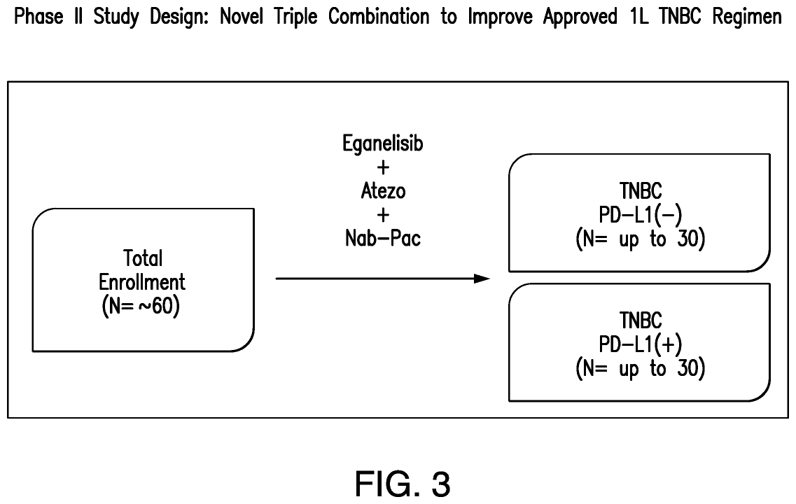

FIG. 3 shows phase II study design: triple combination to improve approved 1L TNBC regimen.

Key Design Features:
A safety run-in evaluated the safety of the triplet of:
   Eganelisib 30 mg oral daily
   nab-pac100 mg/m2 given IV on days 1, 8, & 15
   atezo840 mg given IV on days 1 & 15
   in 28 day cycles
Expansion phase of the phase II study was initiated to enroll approximately 60 patients (30 PD-L1(−) and 30 PD-L1(+)).
Ventana PD-L1 (SP142) assay (cutoff≥1% IC) was used to align with IMpassion130 design.
Primary efficacy endpoint is confirmed complete response (CR) rate per RECIST v1.1; secondary endpoints include the overall response rate (ORR) and safety assessment.
Tumors are assessed every 8 weeks by CT/MRI scan.

Key Eligibility Criteria:
Unresectable locally advanced or metastatic TNBC
No prior systemic therapy for advanced disease
Presence of measurable disease
ECOG performance status 0/1

Demographics and baseline characteristics for evaluable patients are listed in the table below.

| Demographics | N = 13 | | Cancer History | n (%) | | Prior Therapies | n (%) | |
|---|---|---|---|---|---|---|---|---|
| Age, mean ± SD | 55 ± 16 | | Stage at study start | | | Radiotherapy | 5 | (38.5) |
| Female, n (%) | 13 | (100) | II | 1 | (7.7) | Surgery | 10 | (76.9) |
| | | | III | 0 | | | | |
| | | | IV | 11 | (84.6) | | | |
| | | | Unknown | 1 | (7.7) | | | |
| ECOG performance | | | Metastatic sites | | | Systemic therapy* | 8 | (61.5) |
| status, n (%) | | | Liver | 3 | (23.1) | Alkylating agent | 7 | (53.8) |
| 0 | 7 | (53.8) | Lung | 4 | (30.8) | Anthracycline | 7 | (53.8) |
| 1 | 6 | (46.2) | Bone | 5 | (38.5) | Taxane | 7 | (53.8) |

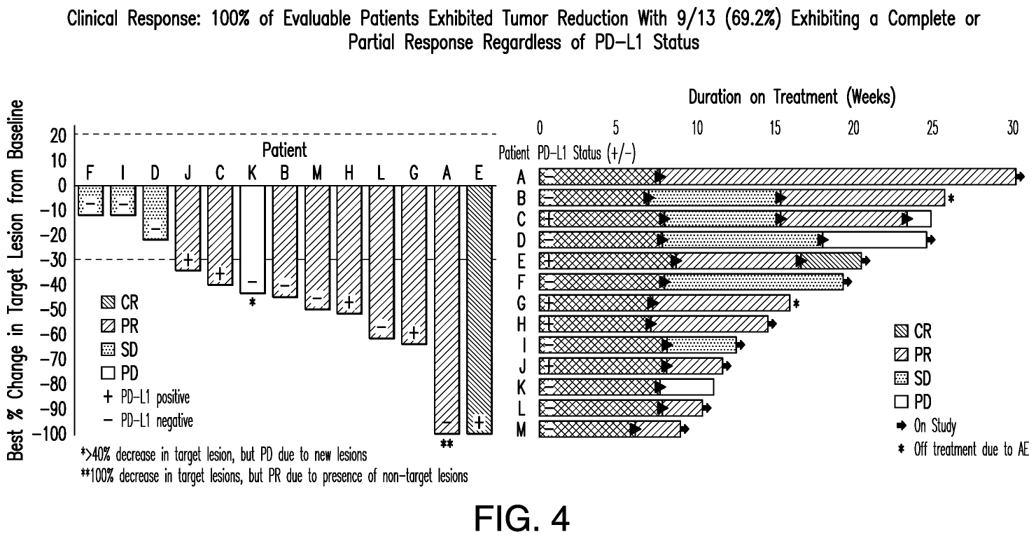

FIG. 4 shows clinical response: 100% of evaluable patients exhibited tumor reduction with 9/13 (69.2%) exhibiting a complete or partial response regardless of PD-L1 status. The results are also listed in the table below.

| Investigator Assessment | Total | | PD-L1(+) | | PD-L1(−) |
|---|---|---|---|---|---|
| Per RECIST 1.1 | N = 13 | | N = 5 | | N = 8 |
| Best Overall Response (BOR)* | | | | | |
| Complete Response (CR), n (%) | 1 | (7.7) | 1 | (20.0) | 0 |
| Partial Response (PR), n (%) | 8 | (61.5) | 4 | (80.0) | 4 (50.0) |
| Stable Disease (SD), n (%) | 3 | (23.1) | 0 | | 3 (37.5) |
| Progressive Disease (PD), n (%) | 1 | (7.7) | 0 | | 1 (12.5) |
| Overall Response Rate (ORR) | 9 | (69.2) | 5 | (100.0) | 4 (50.0) |
| (CR + PR), n (%) | | | | | |

*unconfirmed BOR presented, but 2 PRs of PD-L1(+) and 2 PRs of PD-L1(−) are confirmed.

The safety data are provided in the table below. Safety is in line with expectations of component drugs, no additive or new safety signals.

### Most Common TEAEs (All Grade) in >25% of All Treated Patients (N=14)*

| | TEAE | Immune-related | | TEAE | | Immune-related |
|---|---|---|---|---|---|---|
| Preferred Term | (All) | TEAE (All) | | (≥Gr. 3) | | TEAE (≥Gr. 3) |
| Nausea | 9 (64.3) | 0 | | 0 | | 0 |
| Alopecia | 8 (57.1) | 0 | | 0 | | 0 |
| Rash maculo-papular | 6 (42.9) | 1 | (7.1) | 2 | (14.3) | 0 |
| Diarrhea | 5 (35.7) | 0 | | 2 | (14.3) | 0 |
| Neutrophil count decreased | 5 (35.7) | 0 | | 3 | (21.4) | 0 |
| Alanine aminotransferase increased | 5 (35.7) | 1 | (7.1)** | 1 | (7.1)** | 1 (7.1)** |
| Aspartate aminotransferase increased | 4 (28.6) | 1 | (7.1)** | 1 | (7.1)** | 1 (7.1)** |

*Other TEAEs between 25-30%: decreased appetite, dizziness, peripheral sensory neuropathy, dyspnea, and pruritus. Dose holds of atezo/nab-pac (n = 4) and eganelisib (n = 6) were reported. Four patients discontinued the triplet regimen: 2 for PD and 2 for AE.
**All same patient

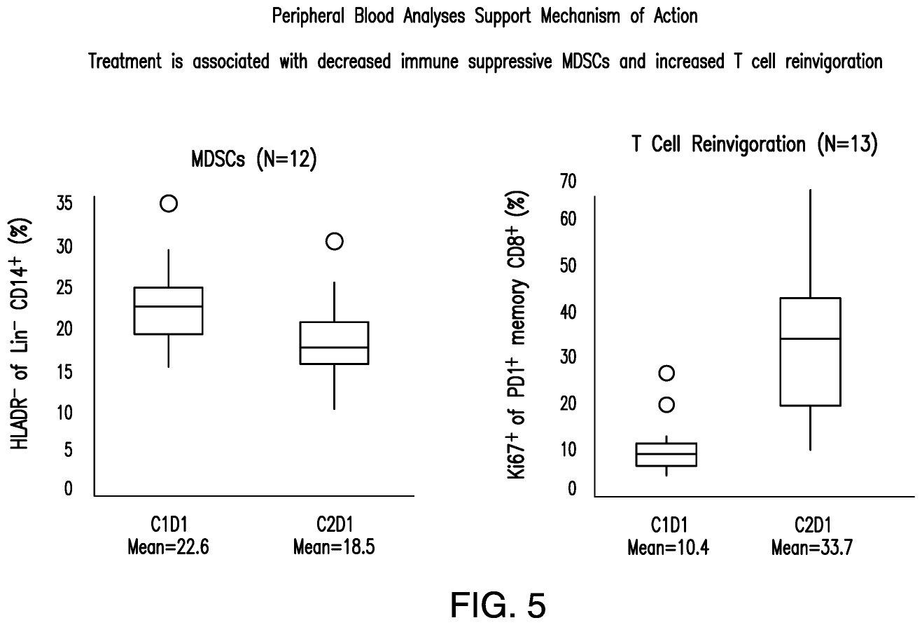

Peripheral blood analyses support mechanism of action (FIG. 5). Treatment is associated with decreased immune suppressive MDSCs and increased T cell reinvigoration.

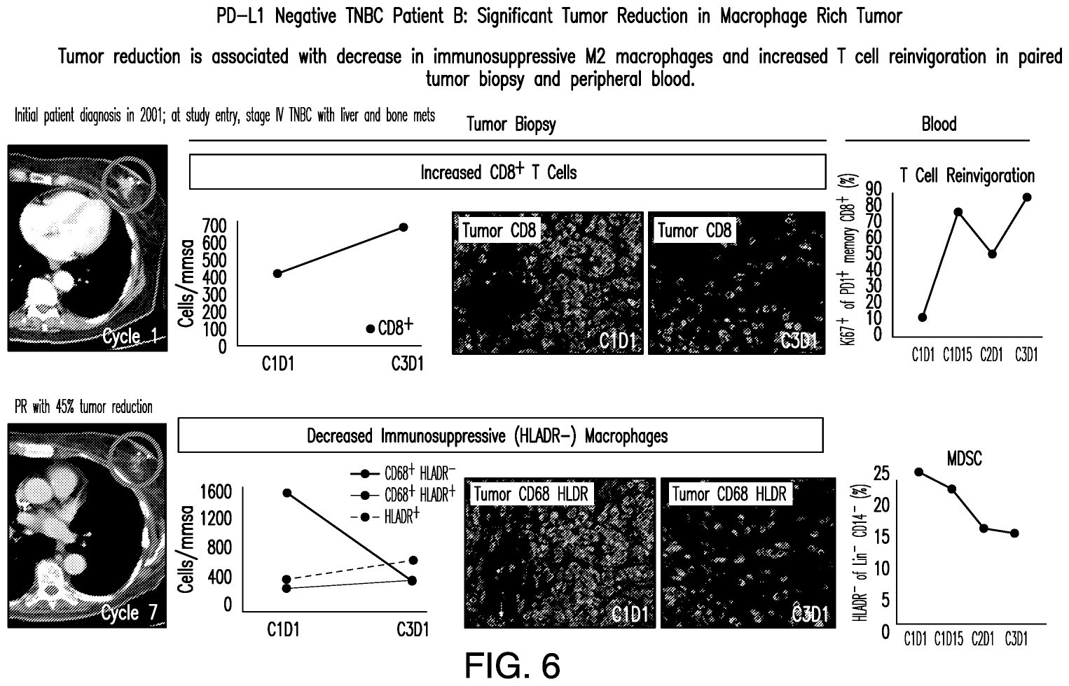

Significant tumor reduction in macrophage rich tumor was observed in PD-L1 negative TNBC patient B (FIG. 6). Tumor reduction is associated with decrease in immunosuppressive M2 macrophages and increased T cell reinvigoration in paired tumor biopsy and peripheral blood.

Conclusion: 100% of evaluable patients experienced tumor reduction and 69.2% of evaluable patients experienced objective responses—a compelling indication of anti-tumor activity, irrespective of PD-L1 status. Translational data show decreased immunosuppressive macrophages/MDSCs and increased immune activation, consistent with the mechanism of action of eganelisib in the triplet regimen. Acceptable safety profile, no additive or new safety signals.

Additional data from the trial (based on data snapshot of Jun. 26, 2021) are provide below:

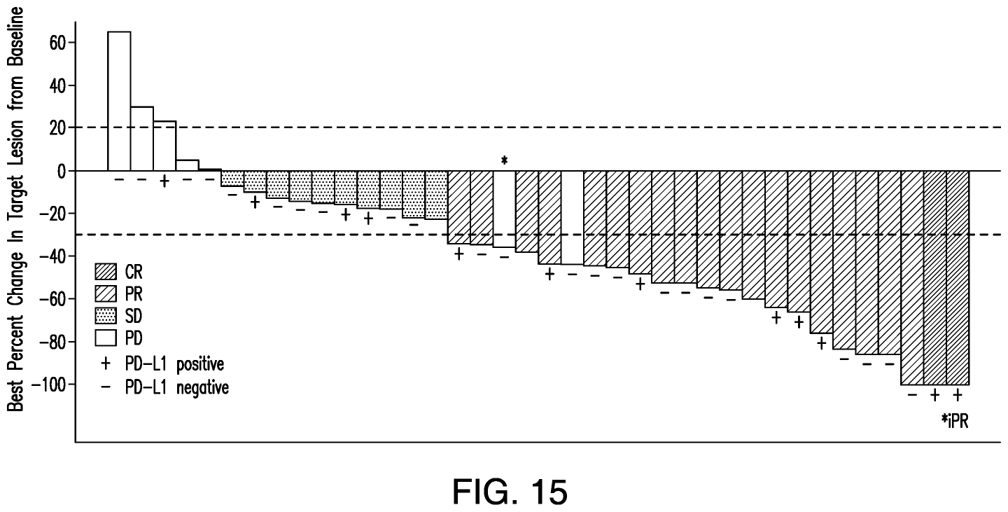

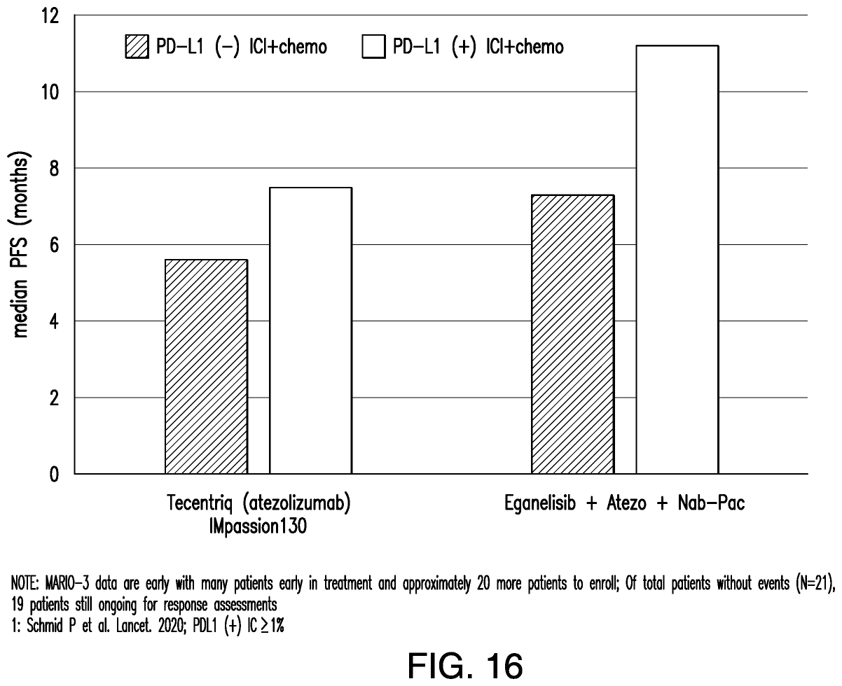

86.8% of evaluable patients achieved tumor reduction (FIG. 15). The preliminary mPFS data for eganelisib+atezolizumab+nab-paclitaxel in both PD-L1(+) and PD-L1(−) patients are shown in FIG. 16, in comparison to the mPFS data in the IMpassion130 trial (Schmid et al., Lancet. 2020). The data are also shown in the table below.

| | Total | | PD-L1(−) | | PD-L1(+) | |
|---|---|---|---|---|---|---|
| | Eganelisib + | | Eganelisib + | | Eganelisib + | |
| | Atezo + Nab-pac | | Atezo + Nab-pac | | Atezo + Nab-pac | |
| Response | N = 38 | | N = 23 | | N = 12 | |
| Median PFS, months | 7.4 | [5.3, NA] | 7.3 | [3.5-NA] | 11.2 | [5.3-11.2] |
| [95% CI] | | | | | | |
| Patients Not Yet | 21 | (55.3) | 11 | (47.8) | 7 | (58.3) |
| Progressed | | | | | | |
| (Potential For PFS | | | | | | |
| Extension), n (%) | | | | | | |
| ORR, n (%) | 21 | (55.3) | 11 | (47.8) | 8 | (66.7) |
| SD, n (%) | 11 | (28.9) | 7 | (30.4) | 3 | (25.0) |
| DCR, n (%) | 32 | (84.2) | 18 | (78.2) | 11 | (91.7) |
| DOR, months | (N = 21) | | (N = 11) | | (N = 8) | |
| [95% CI] | 9.2 | [1.9-9.2] | 5.6 | [1.6-NA] | 9.2 | [1.9-9.2] |

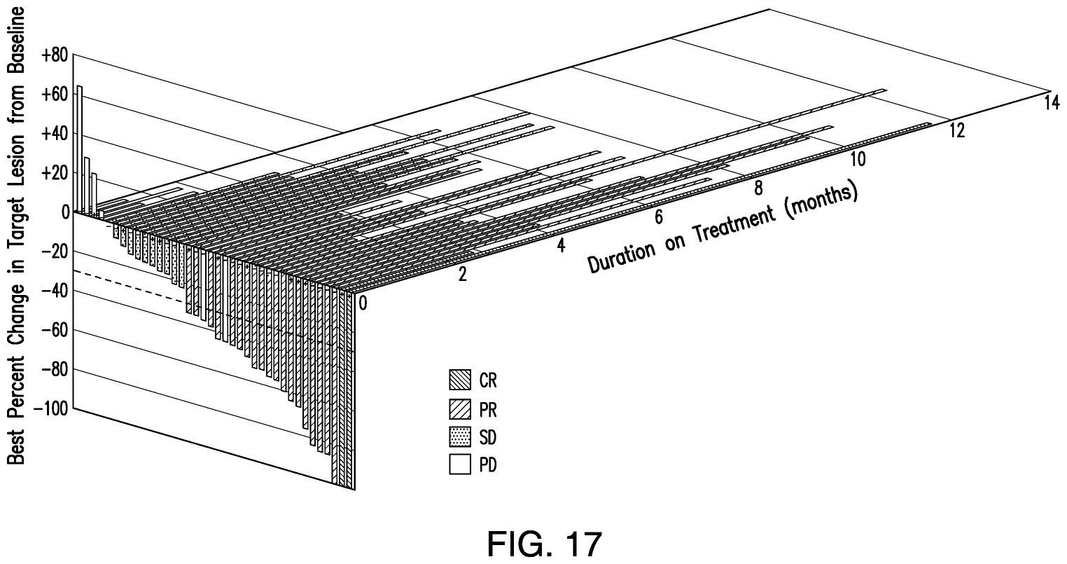

Durable clinical benefit has been observed in patients with SD as well as those with PRs and CRs (FIG. 17), and durability of clinical benefit continues to mature for the majority of treated patients.

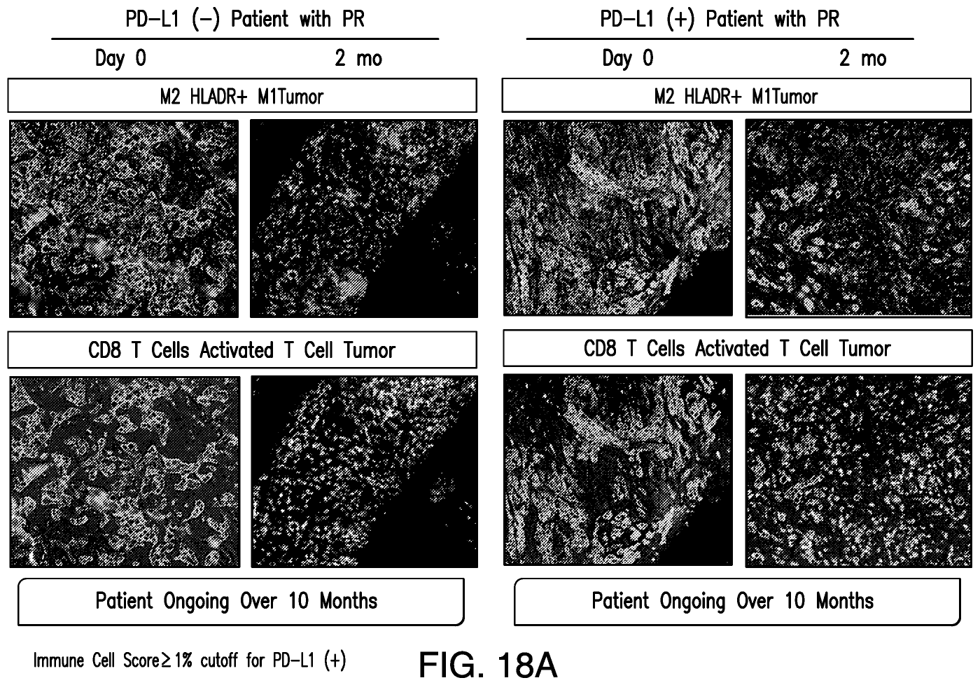

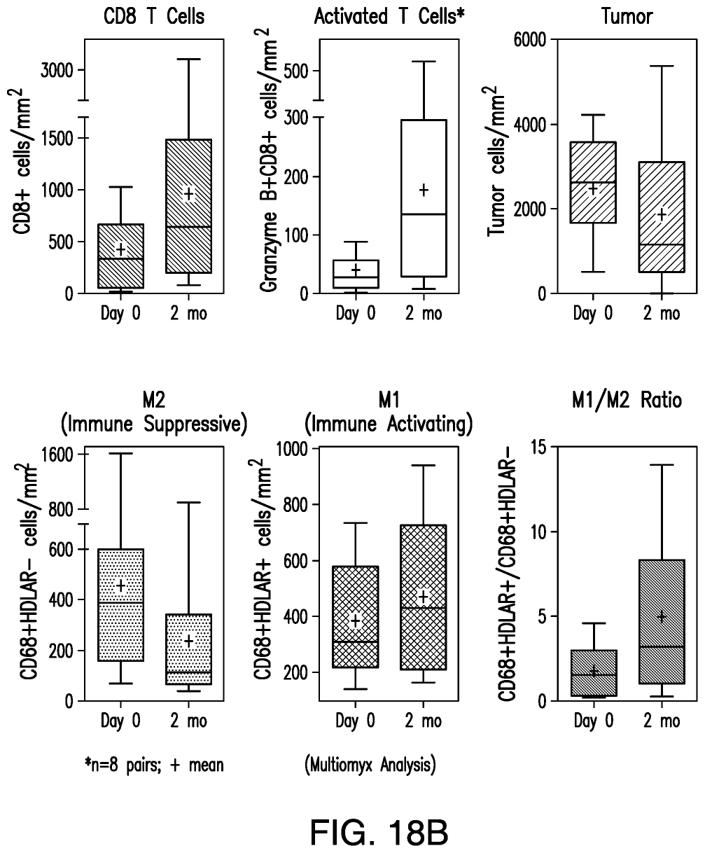

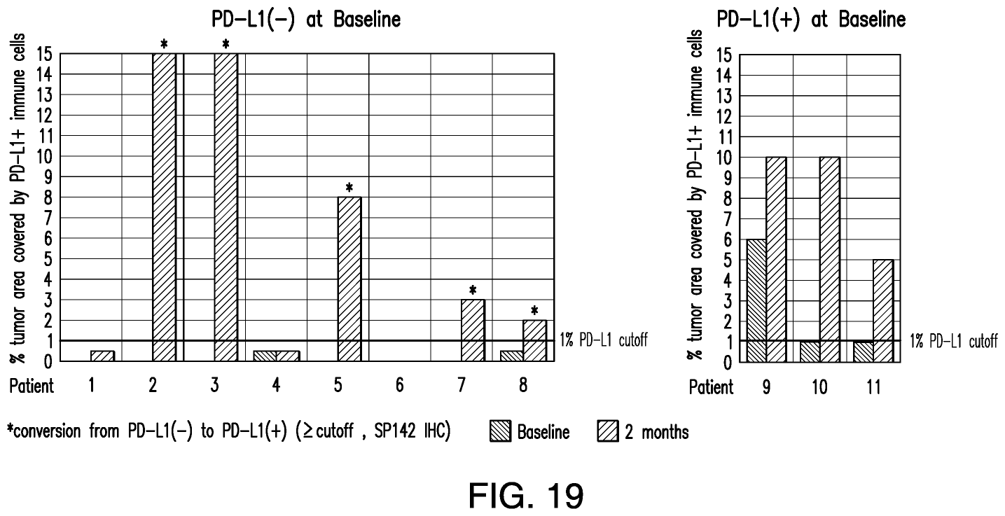

Data quantified in 11 paired tumor biopsies showed reduced immune suppression and increased immune activation regardless of PD-L1 status (FIG. 18A and FIG. 18B). Eganelisib resulted in on-mechanism conversion of patients from PD-L1(−) to PD-L1(+) and increase in PD-L1 expression in PD-L1(+) patients (FIG. 19).

Updated demographics and baseline characteristics for evaluable patients (based on data snapshot of Jun. 26, 2021) are listed in the tables below. A majority of enrolled patients had stage IV disease and had progressed on systemic therapy.

| | TNBC, N = 43 | |
|---|---|---|
| Demographics | | |
| Age, mean ± SD | 58 ± 14 | |
| Gender, n (%) | | |
| Male | 0 | |
| Female | 43 | (100) |
| ECOG performance status, n (%) | | |
| 0 | 26 | (60.5) |
| 1 | 17 | (39.5) |
| Race/ethnicity, n (%) | | |
| White | 35 | (81.4) |
| Hispanic or Latino | 5 | (11.6) |
| Black or African American | 4 | (9.3) |
| Asian | 1 | (2.3) |
| Duration of Eganelisib Exposure, weeks | | |

| | |
|---|---|
| Mean ± SD | 18.3 ± 11.6 |
| Min, Max | 2.4, 48.0 |

| Cancer History | Total | | PD-L1 − | | PD-L1 + | |
|---|---|---|---|---|---|---|
| n (%) | N = 38 | | N = 23 | | N = 12 | |
| Initial diagnosis | | | | | | |
| Local, resectable | 18 | (47.4) | 13 | (56.5) | 3 | (25.0) |
| Locally advanced | 2 | (5.3) | 2 | (8.7) | 0 | |
| Metastatic | 18 | (47.4) | 8 | (34.8) | 9 | (75.0) |
| Stage at study start | | | | | | |
| II | 2 | (5.3) | 2 | (8.7) | 0 | |
| III | 4 | (10.5) | 1 | (4.3) | 3 | (25.0) |
| IV | 26 | (68.4) | 18 | (78.3) | 6 | (50.0) |
| Unknown | 6 | (15.8) | 2 | (8.7) | 3 | (25.0) |
| Metastatic sites | | | | | | |
| Bone | 14 | (36.8) | 12 | (52.2) | 1 | (8.3) |
| Liver | 10 | (26.3) | 7 | (30.4) | 3 | (25.0) |
| Lung | 12 | (31.6) | 7 | (30.4) | 3 | (25.0) |
| Lymph Node | 14 | (36.8) | 7 | (30.4) | 5 | (41.7) |
| Skin | 2 | (5.3) | 2 | (8.7) | 0 | |
| Other | 10 | (26.3) | 6 | (26.1) | 4 | (33.3) |

| Prior Therapies | Total | | PD-L1 − | | PD-L1 + | |
|---|---|---|---|---|---|---|
| n (%) | N = 38 | | N = 23 | | N = 12 | |
| Radiotherapy | 16 | (42.1) | 7 | (30.4) | 7 | (58.3) |
| Surgery | 32 | (84.2) | 20 | (87.0) | 9 | (75.0) |
| Systemic therapy* | 22 | (57.9) | 14 | (60.9) | 6 | (50.0) |
| Alkylating agent | 20 | (52.6) | 13 | (56.5) | 5 | (41.7) |
| Anthracycline | 13 | (34.2) | 9 | (39.1) | 4 | (33.3) |
| Antimetabolite | 6 | (15.8) | 5 | (21.7) | 1 | (8.3) |
| Hormone associated | 6 | (15.8) | 3 | (13.0) | 3 | (25.0) |
| Mitotic inhibitor (taxane) | 18 | (47.4) | 13 | (56.5) | 3 | (25.0) |
| Other | 3 | (7.9) | 2 | (8.7) | 1 | (8.3) |

Updated safety profiles (based on data snapshot of Jun. 26, 2021) are listed in the tables below. No new or additive safety signals were observed in eganelisib combination compared to benchmark trials.

### Most Common TEAEs in ≥25% of All Treated Patients (N=43)

| | | Eganelisib- | | Eganelisib- |
|---|---|---|---|---|
| | | related | | related |
| | TEAE | TEAE | TEAE | TEAE |
| Preferred Term | (All) | (All) | (≥Gr. 3) | (≥Gr. 3) |
| Nausea | 22 (51.2) | 14 (32.6) | 0 | 0 |
| Fatigue | 21 (48.8) | 17 (39.5) | 3 (7.0) | 1 (2.3) |
| Alopecia | 14 (32.6) | 3 (7.0) | 0 | 0 |
| Diarrhea | 14 (32.6) | 11 (25.6) | 3 (7.0) | 1 (2.3) |
| Rash maculo- | 13 (30.2) | 11 (25.6) | 4 (9.3) | 4 (9.3) |
| papular | | | | |
| Alanine | 12 (27.9) | 9 (20.9) | 8 (18.6) | 8 (18.6) |
| aminotransferase | | | | |
| increased* | | | | |
| Aspartate | 11 (25.6) | 10 (23.3) | 6 (14.0) | 6 (14.0) |
| aminotransferase | | | | |
| increased | | | | |

Updated demographics and baseline characteristics for evaluable patients (based on data snapshot of Oct. 2, 2021) are listed in the tables below.

| | TNBC | Cancer History | Total | Prior Therapies | Total |
|---|---|---|---|---|---|
| | N = 50 | n (%) | N = 44 | n (%) | N = 44 |
| Demographics | | Initial diagnosis | | Radiotherapy | 18 (40.9) |
| Age, mean + SD | 58 = 14 | Local, resectable | 19 (43.2) | Surgery | 38 (86.4) |
| Gender, n (%) | | Locally advanced | 2 (4.5) | Systemic therapy* | 24 (54.5) |
| Male | 0 | Metastatic | 22 (50.0) | Alkylating agent | 20 (45.5) |
| Female | 50 (100) | Metastatic sites at | | Anthracycline | 13 (29.5) |
| ECOG performance | | enrollment | | Antimetabolite | 6 (13.6) |
| status, n (%) | | Visceral (V) only | 14 (31.8) | Hormone associated | 7 (15.9) |
| 0 | 28 (56.0) | Non-visceral (NV) only | 16 (36.4) | Mitotic inhibitor (taxane) | 18 (40.9) |
| 1 | 20 (40.0) | Both V + NV | 14 (31.8) | Other | 3 (6.8) |
| Not reported | 2 (4.0) | Bone | 17 (38.6) | | |
| Duration of Eganelisib | | Liver | 14 (31.8) | | |
| Exposure, weeks | | Lung | 18 (40.9) | | |
| Mean ± SD | 18.3 ± 12.5 | Lymph Node | 22 (50.0) | | |
| Min, Max | 1.7, 53.3 | Skin | 2 (4.5) | | |
| | | Other | 11 (25.0) | | |

Updated safety profiles (based on data snapshot of Oct. 2, 2021) are listed in the tables below. 7 patients discontinued treatment for treatment-related TEAEs, including 4 patients for hepatic AE, 2 for peripheral neuropathy and one for rash maculo-popular (note one patient with hepatic AE also had diarrhea and one patient with peripheral neuropathy had pneumontitis). 9 patients had treatment-related SAEs\hat{ }: skin (N=4; dermatitis, hand dermatitis, rash-papular macular, dermatitis on bilateral arms); general disorders (N=3; chills, pyrexia), hepatic (N=3; ALT/AST/transaminases increased); infections (N=2; pneumonia, pharyngitis), gastrointestinal disorders (N=2; diarrhea, vomiting). Single events included supraventriculartachycardia, autoimmune disorder, decreased appetite, Guillain-Barre syndrome, pneumonitis, and flushing.

### Most Common Treatment-Related TEAEs in ≥10% of All Treated Patients** (N=50)

| | Treatment- | Treatment- | Preferred | Treatment- | Treatment- |
|---|---|---|---|---|---|
| Preferred or | related | related | or Grouped | related | related |
| Grouped Term | TEAE (All) | TEAE (≥Gr. 3) | Term | TEAE (All) | TEAE (≥Gr. 3) |
| Nausea | 25 (50.0) | 0 | Peripheral sensory | 9 (18.0) | 3 (6.0) |
| | | | neuropathy | | |
| Fatigue | 24 (48.0) | 3 (6.0) | Decreased appetite | 8 (16.0) | 0 |
| Skin AEs | 18 (36.0) | 6 (12.0) | Headache | 8 (16.0) | 0 |
| Diarrhea | 15 (30.0) | 3 (6.0) | Stomatitis | 7 (14.0) | 0 |
| Hepatic AEs* | 14 (28.0) | 9 (18.0) | Dysgeusia | 7 (14.0) | 0 |
| Alopecia | 13 (26.0) | 0 | Constipation | 6 (12.0) | 0 |
| Vomiting | 11 (22.0) | 1 (2.0) | Weight decreased | 5 (10.0) | 1 (2.0) |
| Neutropenia AEs | 11 (22.0) | 8 (16.0) | Hypokalaemia | 5 (10.0) | 0 |
| Pyrexia | 9 (18.0) | 0 | | | |

*1 Grade 4 and No Hy's Law
** No treatment-related Grade 5 AEs

The mPFS results in patients with both PD-L1(−) and PD-L1(+) tumors are shown in the following table, as compared to IMpassion130 trial.

| | PD-L1(−), | PD-L1(+), |
|---|---|---|
| | median PFS | median PFS |
| | (months) | (months) |
| eganelisib + atezo + nab-pac | 7.3 (N = 27) | 11.0 (N = 14) |
| IMpassion130 (atezo + nab-pac) | 5.6 (N = 266) | 7.5 (N = 185) |

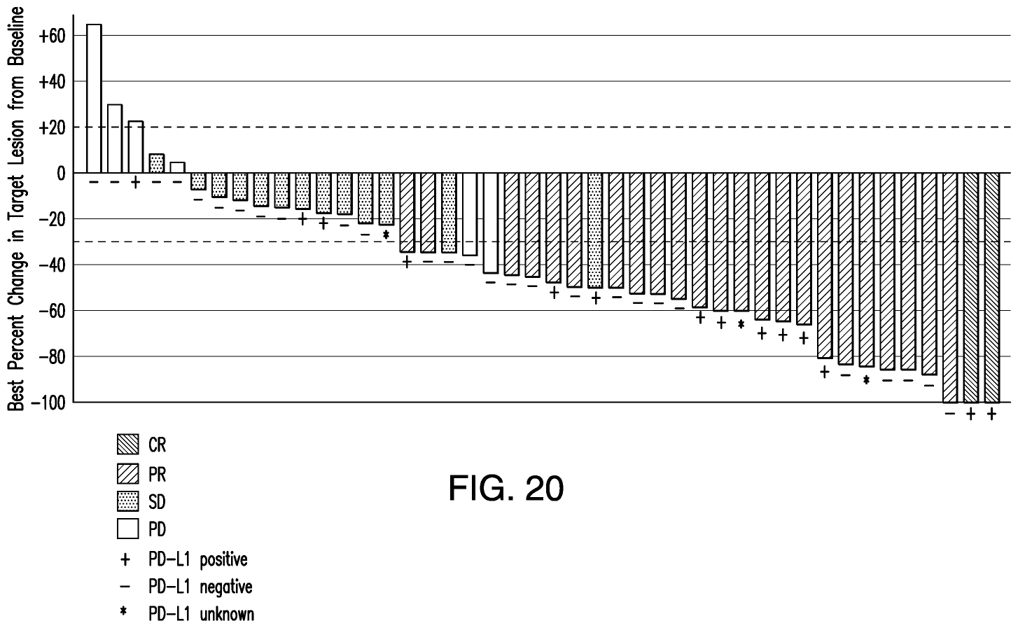

88.6% of evaluable patients achieved tumor reduction (FIG. 20), including tumor reduction in (92.8%) of PD-L1(+) and (85.2%) of PD-L1(−) patients. Immune Cell Score≥1% cutoff for PD-L1(+).

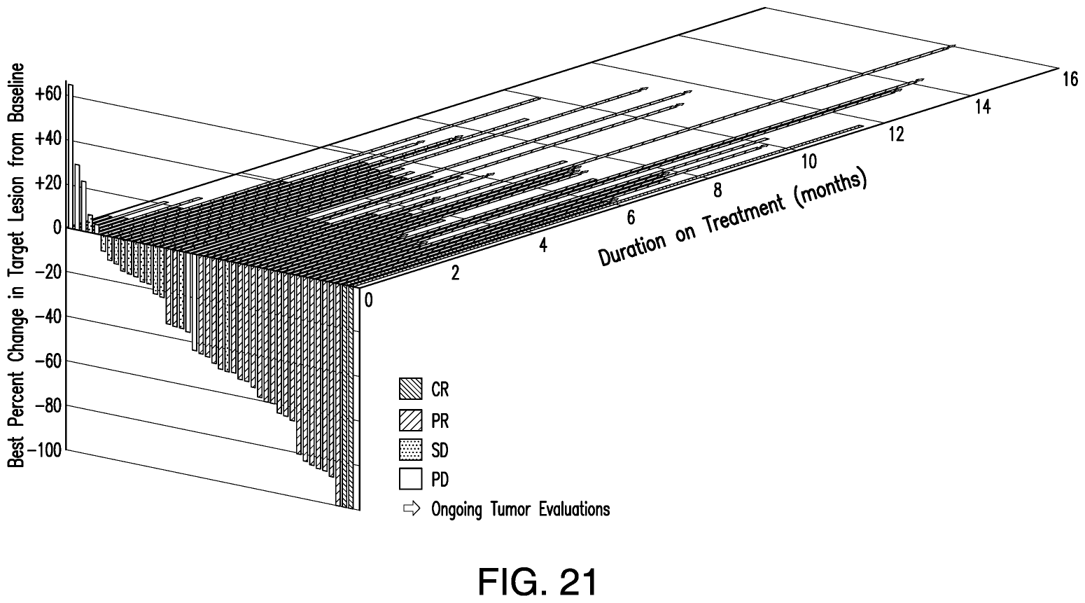

Durable clinical benefit has been observed in patients regardless of baseline PD-L1 (FIG. 21), including 86% disease control rate as shown in the table below.

| | Total | | PD-L1(−) | | PD-L1(+) | |
|---|---|---|---|---|---|---|
| | Eganelisib + | | Eganelisib + | | Eganelisib + | |
| | Atezo + | | Atezo + | | Atezo + | |
| | Nab-pac | | Nab-pac | | Nab-pac | |
| Response | N = 44 | | N = 27 | | N = 14 | |
| Median PFS, | 7.3 | [5.5, NA] | 7.3 | [4.2, NA] | 11.0 | [5.3, NA] |
| months | | | | | | |
| [95% CI] | | | | | | |
| Patients Not Yet | 24 | (54.5) | 13 | (48.1) | 8 | (57.1) |
| Progressed | | | | | | |
| (Potential For PFS | | | | | | |
| Extension), n (%) | | | | | | |
| CR, n (%) | 2 | (4.5) | 0 | (0) | 2 | (14.3) |
| PR, n (%) | 23 | (52.3) | 13 | (48.1) | 8 | (57.1) |
| ORR, n (%) | 25 | (56.8) | 13 | (48.1) | 10 | (71.4) |
| SD, n (%) | 13 | (29.5) | 9 | (33.3) | 3 | (21.4) |
| DCR, n (%) | 38 | (86.3) | 22 | (81.4) | 13 | (92.8) |
| DOR, months | (N = 25) | | (N = 13) | | (N = 10) | |
| [95% CI] | 9.1 | [5.5, NA] | 5.5 | [1.8, NA] | 9.1 | [1.8, NA] |

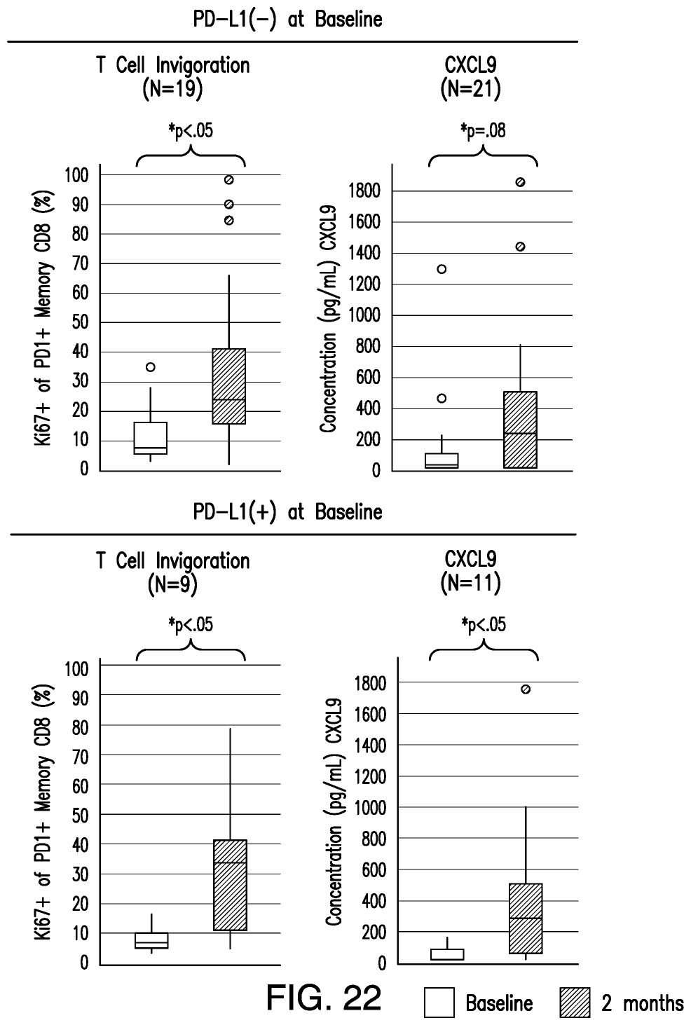

Increased immune activation regardless of baseline PD-L1 status in peripheral blood has been observed (FIG. 22).

### Example 3: Preliminary Analysis of a Phase 2, Multicenter, Randomized, Active-Control Study to Evaluate the Efficacy and Safety of Eganelisib (Compound 1) in Combination with Nivolumab Compared to Nivolumab Monotherapy in Patients with Advanced Urothelial Carcinoma

Inhibition of the PD-1 pathway has demonstrated clinical benefit in metastatic urothelial carcinoma (mUC); however, response rates of 15% to 29% highlight the need for more effective therapies, especially for PD-L1− (negative) patients. Eganelisib is an oral agent which selectively inhibits PI3K-γ, with the goal of improving the immune response to checkpoint inhibitors (CPI).

Methods: Eligible patients (pts) with mUC who progressed on >1 platinum-based chemotherapy regimen and were CPI naïve were enrolled. Pts were randomized 2:1 to receive eganelisib in combination with nivolumab (EN) or placebo with nivolumab (PN). Pts were stratified by baseline circulating monocytic myeloid derived suppressor cells (mMDSC) level. The primary endpoint was objective response rate (ORR) per RECIST v1.1 in pts with high baseline mMDSC levels. Other endpoints included ORR, progression free survival (PFS) and overall survival (OS) in all pts, PD-L1+/−pts and pts with hepatic adverse events (AE).

Results: Provided herein are preliminary data for the first 49 pts with 33 randomized to receive EN and 16 PN. Preliminary ORR/PFS is presented in the table below. Except for the mMDSC high subgroup, ORR and PFS were more favorable in the EN arm compared to the PN arm. The duration of exposure was a median of 15 weeks for EN and 11 for PN. Most common all-Gr AEs (EN vs PN %) were pyrexia (33 v 0), decreased appetite (30 v 19), pruritis (24 v 6), rash (24 v 6), asthenia (21 v 31), and transaminase elevation (21 v 6). Most common Gr≥3 AEs (EN vs PN %) include hepatotoxicity (15 v 0), transaminase elevation (12 v 6), and rash (9 v 0). Eganelisib dose was reduced from 40 to 30 mg, resulting in a reduction of hepatic AEs (see Example 5 for more detail).

Conclusions: Preliminary data demonstrates that the combination of eganelisib and nivolumab was well tolerated with hepatic and skin-related toxicities more common in the EN arm. When compared to PN, the combination demonstrated an improved ORR and PFS, especially in the PD-L1− subset.

| ORR n of N (%) | Eganelisib + | Nivolumab + |
|---|---|---|
| [95% CI] | Nivolumab | Placebo |
| All Patients | 10 of 33 (30.3) | 4 of 16 (25.0) |
| | [16, 49]* | [7, 52] |
| mMDSC ≥ 22.3 | 0 of 7 (0) | 1 of 3 (33.3) |
| | | [1, 91] |
| mMDSC < 22.3 | 10 of 26 (38.5) | 3 of 13 (23.1) |
| | [20, 59]* | [5, 54] |
| PD-L1+ | 4 of 5 (80.0) | 2 of 4 (50.0) |
| | [28, 100] | [7, 93] |
| PD-L1− | 6 of 23 (26.1) | 1 of 7 (14.3) |
| | [10, 48]* | [0, 58] |
| Pts with ≥Gr3 | 6 of 12 (50.0) | 0 of 0 |
| treatment-related | [21, 79]* | |
| hepatic AEs | | |

| PFS median weeks | Eganelisib + | Nivolumab + | HR estimated |
|---|---|---|---|
| [95% CI] | Nivolumab | Placebo | Cox Regression |
| All Patients | 9.1 [8.0, NE] | 8.0 [7.9, 16.4] | 0.79 [0.39, 1.60] |
| PD-L1− | 9.1 [8.0, NE] | 7.9 [2.4, 8.0] | 0.50 [0.19, 1.32] |

*2 patients with pseudo progression (uPD followed by cPR)

### Example 4: Preliminary Analysis of a Phase 2, Multicenter, Randomized, Active-Control Study to Evaluate the Efficacy and Safety of Eganelisib (Compound 1) in Combination with Nivolumab Compared to Nivolumab Monotherapy in Patients with Advanced Urothelial Carcinoma

Background: PD-1 Inhibitors have demonstrated clinical benefit in metastatic urothelial carcinoma (mUC); however, there remains a need for more effective therapies, especially for PD-L1 low patients. As shown in the following table, in CheckMate-275 (a nivolumab monotherapy trial), inferior outcomes were observed in PD-L1 low patients vs PD-L1 high patients in Overall Response Rate, Progression Free Survival and Overall Survival. The same PD-L1 assay and cutoff that was used in CheckMate-275 trial shown below was used in the trial in this example.

| | ORR, % | Median PFS, | Median OS, |
|---|---|---|---|
| CheckMate-275 | (95% CI) | mo. (95% CI) | mo. (95% CI) |
| All (n = 270) | 20 (16-26) | 1.9 (1.9, 2.3) | 8.6 (6.1-11.3) |
| PD-L1 ≥ 1% (n = 124) | 26 (18-34) | 3.5 (1.9, 3.7) | 11.9 (9.1-19.1) |
| PD-L1 < 1% (n = 146) | 16 (10-23) | 1.9 (1.7, 2.0) | 6.0 (4.4-8.1) |

Eganelisib (Compound 1) is an oral agent which selectively inhibits PI3K-γ, with the goal of improving the immune response to checkpoint inhibitors (CPI) particularly in the setting of tumor types less likely to derive benefit from CPIs, including PD-L1 low and MDSC high subset of patients. FIG. 1 shows eganelisib (Compound 1) mechanism of action.

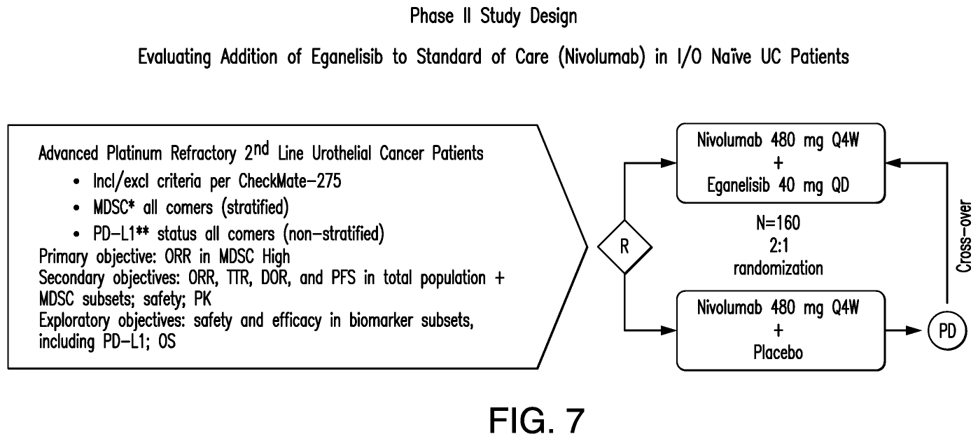

FIG. 7 shows phase II study design to evaluate addition of eganelisib to standard of care (nivolumab) in I/O Naïve UC Patients.

Advanced platinum refractory 2nd line urothelial cancer patients: Inclusion/exclusion criteria per CheckMate-275; MDSC* all corners (stratified); PD-L1** status all corners (non-stratified).
* Circulating mMDSC levels measured in baseline peripheral blood samples based on a Clinical Laboratory Improvement Amendments (CLIA)-certified flow cytometry assay (low [<22.3%] or high [≥22.3%] or ≥/<the median MDSC level of patients in this trial)
** PD-L1 expression measured in baseline/archival tumor biopsies with Dako PD-L1 immunohistochemical 28-8 pharmDx kit approved for nivolumab in UC, except 2 biopsies tested with 22C3 PD-L1 antibody prior to study (Tumor Proportion Score≥1% cutoff for PD-L1 (+))
   Primary objective: ORR in MDSC High
   Secondary objectives: ORR, TTR, DOR, and PFS in total population+MDSC subsets; safety; PK
   Exploratory objectives: safety and efficacy in biomarker subsets, including PD-L1; OS

The dose for nivolumab is 480 mg Q4W. The dose for eganelisib is 40 mg QD.

Patient demographics and baseline characteristics are listed in the table below.

| | Eganelisib + | | Nivolumab + | |
|---|---|---|---|---|
| | Nivolumab | | Placebo | |
| Parameter | (N = 33) | | (N = 16) | |
| Age, mean ± SD | 64.5 ± 8.9 | | 68.1 ± 7.4 | |
| Male, n (%) | 24 | (72.7) | 12 | (75.0) |
| Primary tumor location, n (%) | | | | |
| Urinary bladder | 24 | (72.7) | 13 | (81.3) |
| Renal pelvis | 5 | (15.2) | 2 | (12.5) |
| Ureter | 2 | (6.1) | 1 | (6.3) |
| Urethra | 2 | (6.1) | 0 | |
| ECOG performance status, n (%) | | | | |
| 0 | 20 | (60.6) | 5 | (31.3) |
| 1 | 13 | (39.4) | 11 | (68.8) |
| Prior Systemic Therapies, median (range) | 2.0 | (1, 6) | 2.0 | (1, 5) |
| Liver metastases, n (%) | 8 | (24.2) | 5 | (31.3) |
| MDSC level, n (%) | | | | |
| Low (<22.3) | 26 | (78.8) | 13 | (81.3) |
| High (≥22.3) | 7 | (21.2) | 3 | (18.8) |
| PD-L1 Status, n (%) | | | | |
| ≥1% | 5 | (15.2) | 5 | (31.3) |
| <1% | 23 | (69.7) | 7 | (43.8) |
| Unknown | 5 | (15.2) | 4 | (25.0) |

The dose of eganelisib was reduced from 40 mg QD to 30 mg QD during the trial. The dose reduction decreased the number of reversible liver enzyme elevations.

Patient disposition and exposure are listed in the table below.

| | Eganelisib + | | Nivolumab + | |
|---|---|---|---|---|
| | Nivolumab | | Placebo | |
| Parameter | (N = 33) | | (N = 16) | |
| Duration of exposure, | 15.9 | (2, 45) | 11.1 | (2, 60) |
| median weeks | | | | |
| (min, max) | | | | |
| Eganelisib/placebo | 31.5 | (17.4, 39.9) | 38.6 | (14.2, 40.0) |
| median average daily | | | | |
| dose mg, (min, max) | | | | |
| Ongoing treatment, n (%) | 8 | (24.2) | 5 | (31.3) |
| Discontinued | 25 | (75.8) | 11 | (68.8) |
| treatment, n (%) | | | | |
| Progression of disease | 12 | (48.0) | 4 | (36.4) |
| Adverse event, | 6 | (24.0) | 0 | (0) |
| related to treatment | | | | |
| Adverse event, | 2 | (8.0) | 1 | (9.1) |
| unrelated to treatment | | | | |
| Death | 3 | (12.0) | 3 | (27.3) |
| Investigator decision | 1 | (4.0) | 2 | (18.2) |
| Voluntary withdrawal | 1 | (4.0) | 1 | (9.1) |

Safety: the data show that combination of eganelisib and nivolumab was well tolerated at 30 mg QD dose.
Treatment-Emergent AEs (>20% Nivolumab+Eganelisib)

| | Eganelisib + Nivolumab (N = 33), n (%) | | | Nivolumab + Placebo (N = 16), n (%) | | |
|---|---|---|---|---|---|---|
| | All | Related to | Related to | All | Related to | Related to |
| Preferred Term | Causality | Eganelisib | Nivolumab | Causality | Placebo | Nivolumab |
| Pyrexia | 11 (33.3) | 4 (12.1) | 2 (6.1) | 0 | 0 | 0 |
| Decreased | 10 (30.3) | 7 (21.2) | 5 (15.2) | 4 (25.0) | 1 (6.3) | 0 |
| appetite | | | | | | |
| Disease | 8 (24.2) | 1 (3.0) | 1 (3.0) | 6 (37.5) | 0 | 0 |
| progression | | | | | | |
| Pruritus | 8 (24.2) | 5 (15.2) | 8 (24.2) | 1 (6.3) | 0 | 0 |
| Rash | 8 (24.2) | 7 (21.2) | 4 (12.1) | 1 (6.3) | 0 | 0 |
| Alanine | 8 (24.2) | 7 (21.2) | 7 (21.2) | 2 (12.5) | 0 | 0 |
| aminotransferase | | | | | | |
| increased | | | | | | |
| Asthenia | 7 (21.2) | 6 (18.2) | 5 (15.2) | 4 (25.0) | 2 (12.5) | 1 (6.3) |

Grade ≥3\hat{ } TEAEs (>10% in Nivolumab + Eganelisib)

| | Eganelisib + Nivolumab (N = 33), n (%) | | | Nivolumab + Placebo (N = 16), n (%) | | |
|---|---|---|---|---|---|---|
| | All | Related to | Related to | All | Related to | Related to |
| Preferred Term | Causality | Eganelisib | Nivolumab | Causality | Placebo | Nivolumab |
| Disease | 8 (24.2) | 1 (3.0) | 1 (3.0) | 6 (37.5) | 0 | 0 |
| progression | | | | | | |
| Hepatotoxicity* | 5 (15.2) | 5 (15.2) | 5 (15.2) | 0 | 0 | 0 |
| Alanine | 4 (12.1) | 4 (12.1) | 4 (12.1) | 0 | 0 | 0 |
| aminotransferase | | | | | | |
| increased** | | | | | | |
| Aspartate | 4 (12.1) | 4 (12.1) | 4 (12.1) | 1 (6.3) | 0 | 0 |
| aminotransferase | | | | | | |
| increased* | | | | | | |

\hat{ }No Grade 5
*1 Grade 4
**2 Grade 4

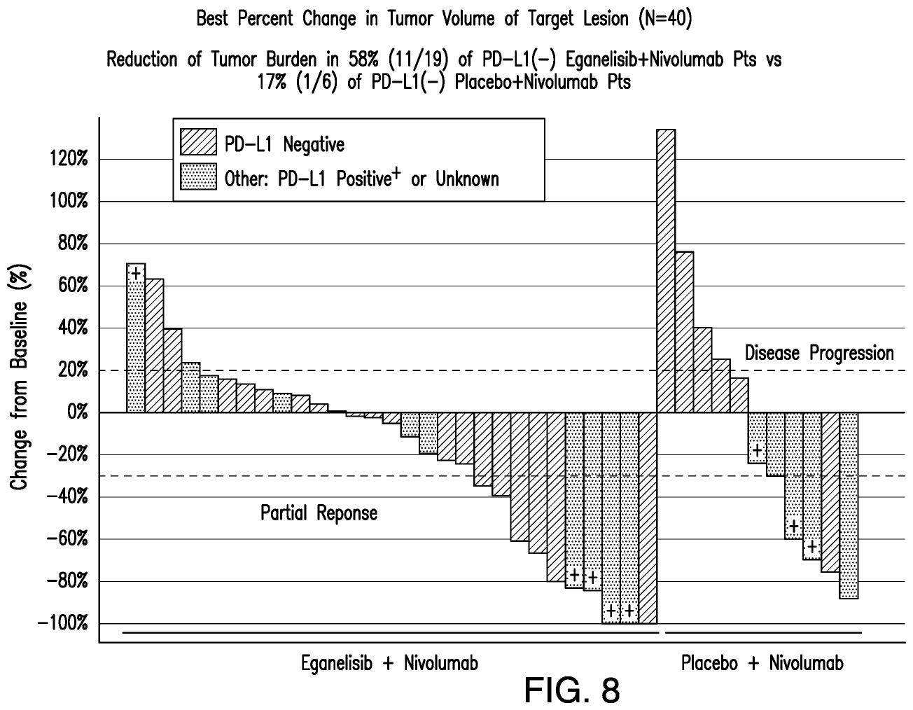

Results: FIG. 8 shows best percent change in tumor volume of target lesion (N=40). Reduction of tumor burden in 58 (11/19) of PD-L1(−) eganelisib+nivolumab patients vs 17% (1/6) of PD-L1(−) placebo+nivolumab patients.

In this trial, 75% (30 of 40 evaluable for PD-L1 status) of patients were PD-L1(−). In CheckMate-275, 54=(143 of1265 evaluable for PD-L1 status) ofpatients were PD-L1(−).

Best overall RECIST response is listed in the table below.

| | All Patients | | | | PD-L1 (−) Patients | | | | PD-L1 (+) Patients | | | |
|---|---|---|---|---|---|---|---|---|---|---|---|---|
| | Eganelisib + | | Nivo + | | Eganelisib + | | Nivo + | | Eganelisib + | | Nivo + | |
| | Nivo | | Placebo | | Nivo | | Placebo | | Nivo | | Placebo | |
| | N = 33 | | N = 16 | | N = 23 | | N = 7 | | N = 5 | | N = 5 | |
| | n | % | n | % | n | % | n | % | n | % | n | % |
| CR | 4* | 12% | 1* | 6% | 2 | 9% | 0 | 0% | 2 | 40% | 0 | 0% |
| PR | 6** | 18% | 3 | 19% | 4 | 17% | 1 | 14% | 2 | 40% | 2 | 40% |
| ORR\hat{ } | 10 | 30% | 4 | 25% | 6 | 26% | 1 | 14% | 4 | 80% | 2 | 40% |
| SD | 8** | 24% | 1 | 6% | 7 | 30% | 0 | 0% | 0 | 0% | 1 | 20% |
| DCR\hat{ }\hat{ } | 18 | 55% | 5 | 31% | 13 | 57% | 1 | 14% | 4 | 80% | 3 | 60% |
| PD | 11 | 33% | 7 | 44% | 6 | 26% | 5 | 71% | 1 | 20% | 1 | 20% |
| NE | 4 | 12% | 4 | 25% | 4 | 17% | 1 | 14% | 0 | 0% | 1 | 20% |

*Confirmed CR
**4 patients had PD at C3, then PR or SD at later cycles
\hat{ }Unconfirmed overall response rate (ORR) (CR + PR)
\hat{ }\hat{ }Unconfirmed disease control rate (DCR) (CR, PR + SD)
Note:
Patients were stratified by MDSC level, but there was no meaningful difference between the DCR in the MDSC high combination arm (29%, n = 7) versus the MDSC high control arm (33%, n = 3)

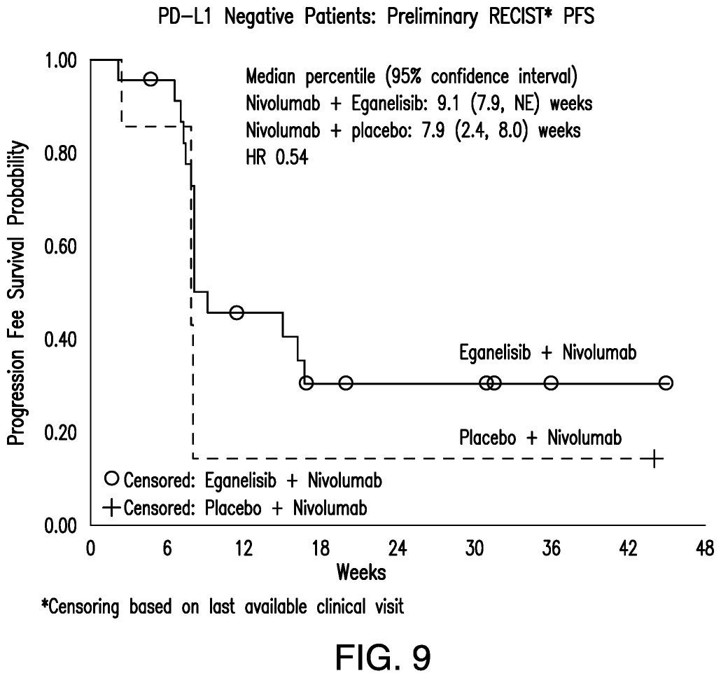

Preliminary progression free survival results are shown in FIG. 9 and the table below. PFS Subgroups by PD-L1 Status: Extended mPFS of 9.1 wks in PD-L1(−) Pts vs 7.9 wks in PD-L1(+) Pts

| | PD-L1(−) | | PD-L1(+) | | ITT | |
|---|---|---|---|---|---|---|
| Eganelisib + | N = 23 | | N = 5 | | N = 33 | |
| Nivolumab | | | | | | |
| Median PFS (wks) | 9.1 | [7.9, NE] | NE | [7.9, NE] | 9.1 | [8.0, 22.3] |
| [95% CI] | | | | | | |
| Patients with | 15 | (65.2) | 2 | (40.0) | 22 | (66.7) |
| Events, n (%) | | | | | | |
| Placebo + | N = 7 | | N = 5 | | N = 16 | |
| Nivolumab* | | | | | | |
| Median PFS (wks) | 7.9 | [2.4, 8.0] | 16.4 | [6.9, NE] | 8.5 | [7.9, 16.4] |
| [95% CI] | | | | | | |
| Patients with | 6 | (85.7) | 3 | (60.0) | 12 | (75.0) |
| Events, n (%) | | | | | | |
| Hazard Ratio | 0.54 | [0.21, 1.43] | NE | | 0.83 | [0.41, 1.67] |
| [95% CI] | | | | | | |

*this trial's placebo + nivolumab median PFS consistent with CheckMate-275 nivolumab monotherapy median PFS:
PD-L1(−) CheckMate-275 median PFS 1.9 mos (8.2 wks) vs. this trial (7.9 wks)
PD-L1(+) CheckMate-275 median PFS 3.5 mos (15.2 wks) vs. this trial (16.4 wks)

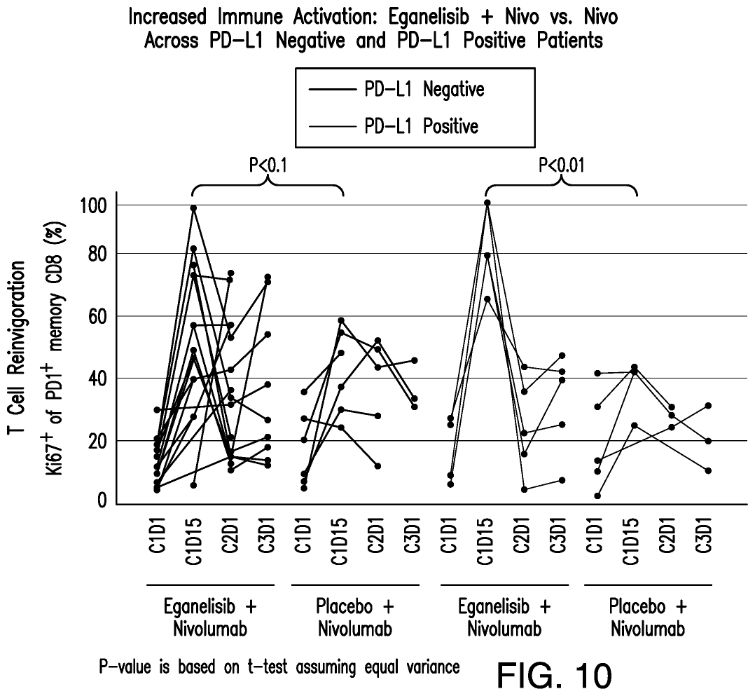

Translational data showed increased immune activation for eganelisib+nivolumab vs. nivolumab across PD-L1 negative and PD-L1 positive patients (FIG. 10).

Conclusion: Preliminary data demonstrates that the combination of eganelisib at 30 mg+nivolumab was well tolerated. The combination demonstrated an improved ORR, DCR and PFS versus nivolumab, especially in PD-L1 negative patients, which represent approximately 70% of the combination arm patients. Translational data demonstrate increased immune activation with eganelisib+nivolumab versus nivolumab, including in PD-L1 low patients. Given the magnitude of unmet need and the magnitude of the benefit seen in PD-L1 low mUC patients, further study in the PD-L1 low mUC population is being planned.

Additional data from the trial (based on data snapshot of Jun. 26, 2021) are provide below:

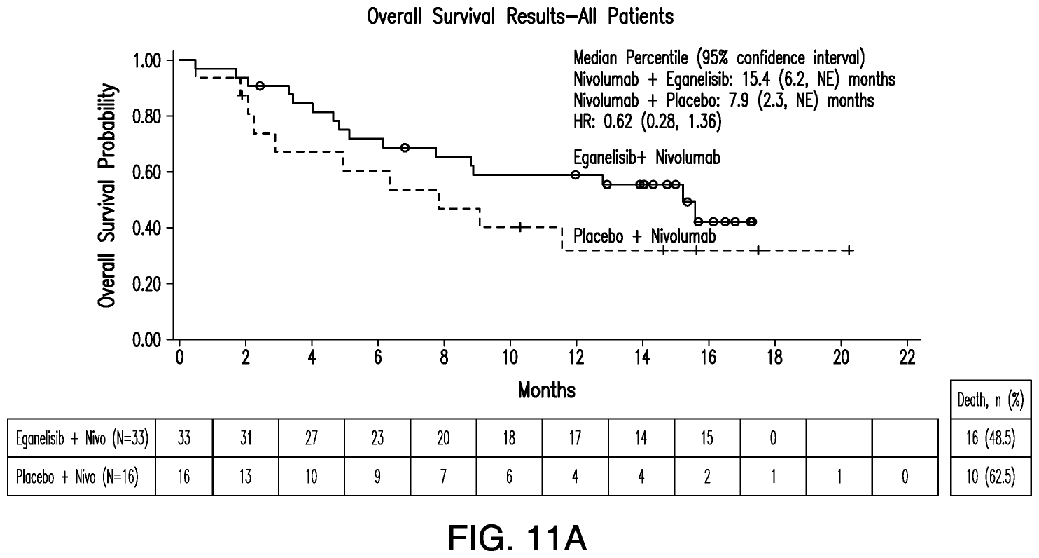

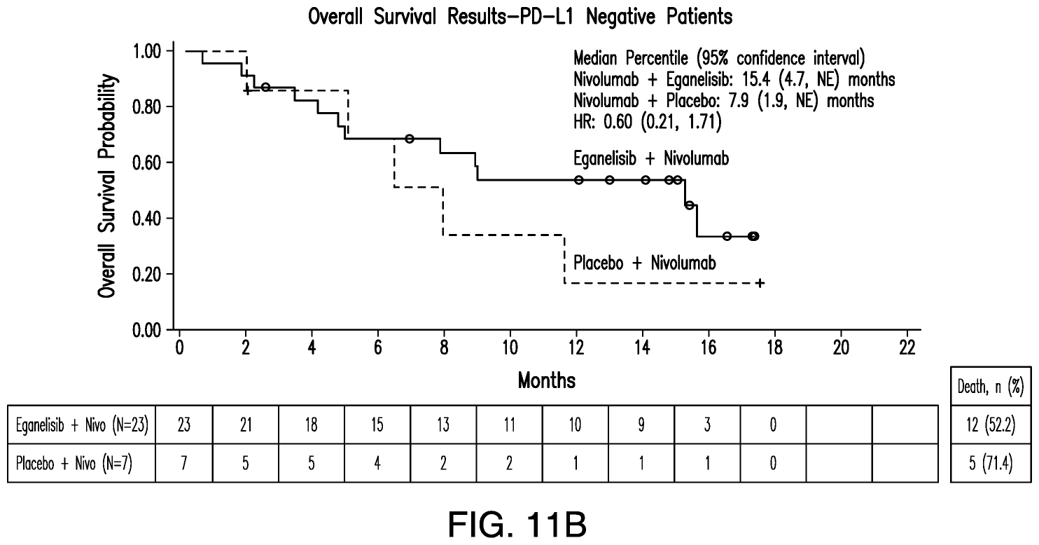

In all patients, combination arm median OS (mOS) was 15.4 months vs 7.9 months on control arm, with hazard ratio (HR) of 0.62 indicating 38% reduction in risk of death (FIG. 11A). At one year landmark, the overall survival probability was 59% in combination vs. 32% in nivolumab. In PD-L1 negative patients, combo arm mOS was 15.4 months vs 7.9 months on control arm, with HR of 0.60 indicating 40% reduction of risk of death (FIG. 11B). At one year landmark, the overall survival probability was 54% in combination vs. 17% in nivolumab.

Best Overall Response per iRECIST is listed in the table below.

| | All Patients | | | | PD-L1 (−) Patients | | | | PD-L1 (+) Patients | | | |
|---|---|---|---|---|---|---|---|---|---|---|---|---|
| | Eganelisib + | | Nivo + | | Eganelisib + | | Nivo + | | Eganelisib + | | Nivo + | |
| | Nivo | | Placebo | | Nivo | | Placebo | | Nivo | | Placebo | |
| | N = 33 | | N = 16 | | N = 23 | | N = 7 | | N = 5 | | N = 5 | |
| | n | % | n | % | n | % | n | % | n | % | n | % |
| CR/iCR | 4 | 12% | 1 | 6% | 2 | 9% | 0 | 0% | 2 | 40% | 0 | 0% |
| PR/iPR | 5 | 15% | 3 | 19% | 3 | 13% | 1 | 14% | 2 | 40% | 2 | 40% |
| iORR | 9 | 27% | 4 | 25% | 5 | 22% | 1 | 14% | 4 | 80% | 2 | 40% |
| SD/iSD | 8 | 24% | 1 | 6% | 7 | 30% | 0 | 0% | 0 | 0% | 1 | 20% |
| iDCR | 17 | 51% | 5 | 31% | 12 | 52% | 1 | 14% | 4 | 80% | 3 | 60% |
| iCPD | 6 | 18% | 3 | 19% | 3 | 13% | 2 | 29% | 1 | 20% | 0 | 0% |
| iUPD/PD | 6 | 18% | 5 | 31% | 4 | 17% | 3 | 43% | 0 | 0% | 1 | 20% |
| NE | 4 | 12% | 3 | 19% | 4 | 17% | 1 | 14% | 0 | 0% | 1 | 20% |

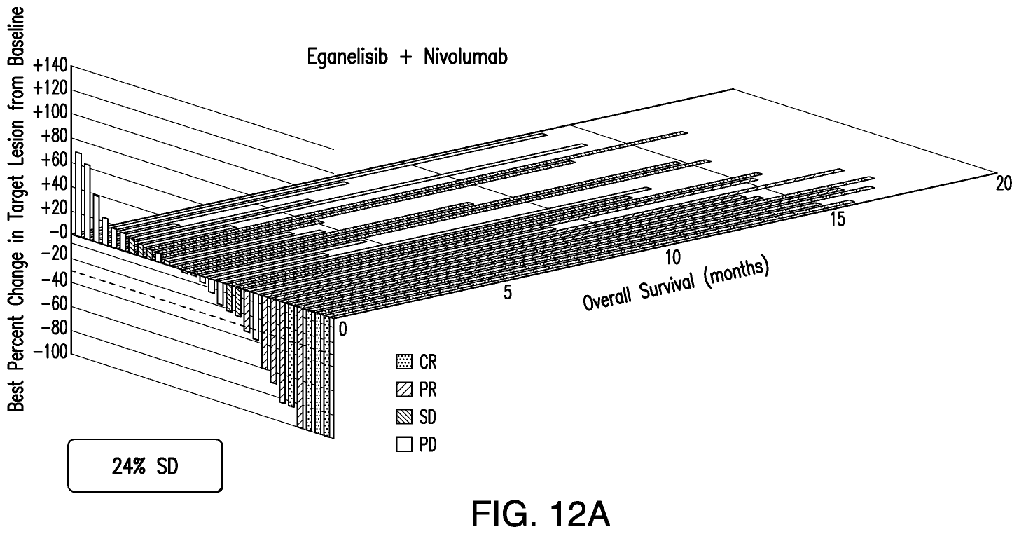

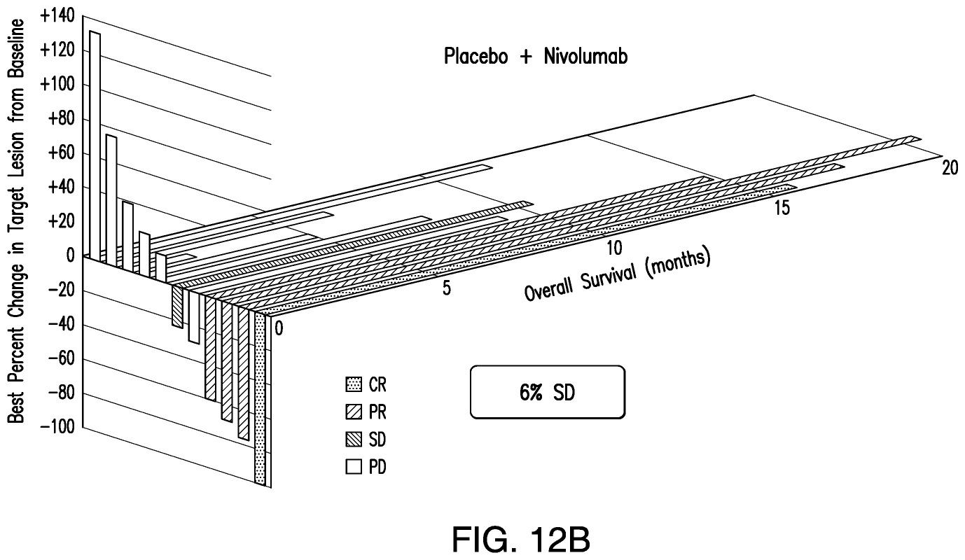

The data showed stable disease contributed to overall survival. FIG. 12A and FIG. 12B show OS benefit in patients with SD as well as those with PRs and CRs. In addition, durability of OS benefit continued to mature for 51% of patients on combination treatment arm vs 18% on the nivolumab control arm.

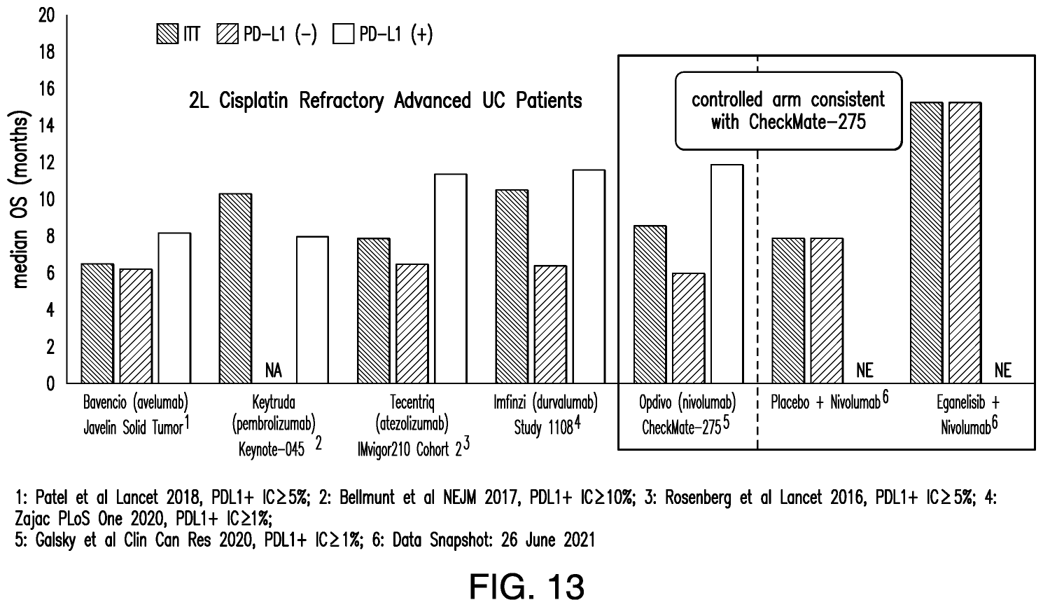

Benchmark second line (2L) urothelial carcinoma (UC) studies result in median OS of 6-12 months with outcomes varying by PD-L1 expression status. See, Patel et al., Lancet 2018; Bellmunt et al. NEJM 2017; Rosenberg et al., Lancet 2016; Zajac, PLoS One 2020; Galsky et al., Clin Can Res 2020. In this study, second line UC patients treated with eganelisib plus nivolumab showed mOS 50-100% greater than benchmark 2L studies (FIG. 13).

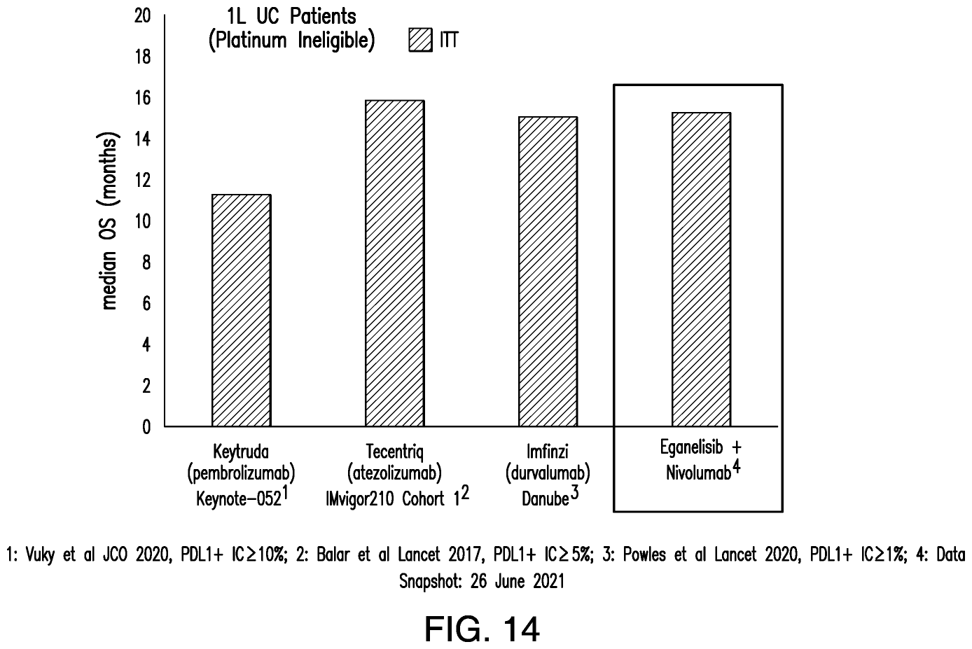

CPIs in 1L mUC patients have median OS of 11-16 months. See, Vuky et al., JCO 2020; Balar et al., Lancet 2017; Powles et al., Lancet 2020. Eganelisib plus nivolumab combination mOS in 2L is comparable (FIG. 14).

Greater immune activation (higher gene set Enrichment Scores and lower P values for pro-inflammatory pathways) with eganelisib+nivolumab combination was observed compared to nivolumab alone regardless of PD-L1 status in peripheral blood. In all patients (day 15 vs. day 0): interferon gamma pathway: ES=0.81, p<0.01 (combination, n=31) vs. ES=0.51, p=0.28 (nivolumab, n=14); interferon alpha pathway: ES=0.87, p<0.01 (combination, n=31) vs. ES=0.53, p=0.39 (nivolumab, n=14). In PD-L1 negative patients (day 15 vs. day 0): interferon gamma pathway: ES=0.82, p<0.01 (combination, n=21) vs. ES=0.55, p=0.15 (nivolumab, n=5); interferon alpha pathway: ES=0.88, p<0.01 (combination, n=21) vs. ES=0.53, p=0.35 (nivolumab, n=5).

The updated patient disposition and exposure information (based on data snapshot of Jun. 26, 2021) are listed in the table below.

| | Eganelisib + | | Placebo + | |
|---|---|---|---|---|
| | Nivolumab | | Nivolumab | |
| Parameter | (N = 33) | | (N = 16) | |
| Duration of exposure, | 16.1 | (2, 75) | 11.1 | (2, 84) |
| median weeks (min, max) | | | | |
| Eganelisib/placebo | 29.9 | (12.2, 39.9) | 34.6 | (14.2, 40.0) |
| median average | | | | |
| daily dose mg, (min, max) | | | | |
| Ongoing treatment, n (%) | 6 | (18.2) | 3 | (18.8) |
| Discontinued | 27 | (81.8) | 13 | (81.3) |
| treatment, n (%) | | | | |
| Progression of disease | 11 | (40.7) | 5 | (38.5) |
| Adverse event, | 8 | (29.6) | 3 | (23.1) |
| related to treatment | | | | |
| Adverse event, | 3 | (11.1) | 0 | |
| unrelated to treatment | | | | |
| Investigator decision | 3 | (11.1) | 2 | (15.4) |
| Death | 1 | (3.7) | 2 | (15.4) |
| Voluntary withdrawal | 1 | (3.7) | 1 | (7.7) |

The updated safety profiles (based on data snapshot of Jun. 26, 2021) are listed in the tables below. In 2L UC, the combination of eganelisib and nivolumab has a favorable safety profile with ≤15% immune-related Grade≥3 TEAEs.

### Treatment-Emergent AEs in ≥25% Patients in the Nivolumab+Eganelisib Arm

| | Eganelisib + Nivolumab (N = 33) | | | Placebo + Nivolumab (N = 16) | | |
|---|---|---|---|---|---|---|
| | All | Related to | Related to | All | Related to | Related to |
| Preferred Term | Causality | Eganelisib | Nivolumab | Causality | Placebo | Nivolumab |
| Pyrexia | 11 (33.3) | 4 (12.1) | 2 (6.1) | 0 | 0 | 0 |
| Decreased | 10 (30.3) | 7 (21.2) | 5 (15.2) | 5 (31.3) | 1 (6.3) | 0 |
| appetite | | | | | | |
| Disease | 9 (27.3) | 0 | 0 | 4 (25.0) | 0 | 0 |
| progression | | | | | | |
| Asthenia | 9 (27.3) | 6 (18.2) | 5 (15.2) | 5 (31.3) | 2 (12.5) | 1 (6.3) |
| Pruritus | 9 (27.3) | 6 (18.2) | 9 (27.3) | 1 (6.3) | 0 | 0 |
| Rash | 9 (27.3) | 8 (24.2) | 5 (15.2) | 1 (6.3) | 0 | 0 |

### Grade≥3 TEAEs in ≥10% Patients in the Nivolumab+Eganelisib Arm

| | Eganelisib + Nivolumab (N = 33) | | | Placebo + Nivolumab (N = 16) | | |
|---|---|---|---|---|---|---|
| | All | Related to | Related to | All | Related to | Related to |
| Preferred Term | Causality | Eganelisib | Nivolumab | Causality | Placebo | Nivolumab |
| Disease | 9 (27.3) | 0 | 0 | 4 (25.0) | 0 | 0 |
| progression | | | | | | |
| Hepatotoxicity* | 5 (15.2) | 5 (15.2) | 5 (15.2) | 0 | 0 | 0 |
| Alanine | 4 (12.1) | 4 (12.1) | 4 (12.1) | 1 (6.3) | 1 (6.3) | 1 (6.3) |
| aminotransferase | | | | | | |
| increased** | | | | | | |
| Aspartate | 4 (12.1) | 4 (12.1) | 4 (12.1) | 1 (6.3) | 1 (6.3) | 1 (6.3) |
| aminotransferase | | | | | | |
| increased* | | | | | | |
| Anemia | 4 (12.1) | 1 (3.0) | 1 (3.0) | 1 (6.3) | 0 | 0 |

### Example 5: Adjustment of Eganelisib Dose

In a phase 1 trial, the opening of expansion cohorts (Parts E, F, and G) was dependent on the determination of the recommended Phase 2 dose for the combination of eganelisib with nivolumab (240 mg Q2W) from the dose escalation cohort (Part C). Following a review of the data from the subjects enrolled at all eganelisib dose levels in Part C (20, 30, and 40 mg QD), including the combination therapy safety profile (AEs, SAEs, and DLTs), the pharmacokinetics of eganelisib, and the exposure-response relationship between eganelisib and PI3K-γ inhibition, the eganelisib dose of 40 mg QD was chosen as the recommended Phase 2 dose in combination with 240 mg Q2W nivolumab.

In a phase 2 trial, for the combination arm, eganelisib was administered QD with intravenous nivolumab given every 4 weeks at 480 mg. At the start of phase 2 trial, the dose of eganelisib was 40 mg QD. Hepatic TEAEs were observed at 40 mg QD in >20% of subjects, while Grade 3 hepatic TEAE were observed in <20% of subjects receiving eganelisib 30 mg QD.

For safety, as shown in the following table, a dose-response relationship appeared, with higher doses of eganelisib associated with higher incidences of hepatic AEs.

| | Eganelisib | | | |
|---|---|---|---|---|
| | Starting Dose | | Patients | Patients |
| | in Combination | | with ≥Grade | with ≥Grade |
| | with | | 3 Hepatic | 3 Hepatic |
| Study | Nivolumab | Patients (n) | TEAE (n) | TEAE (%) |
| Phase 1 | 30 mg | 12 | 1 | 8.3 |
| Part C | | | | |
| Phase 1 | 40 mg | 12 | 5 | 41.7 |
| Part C | | | | |
| Phase 2 | 30 mg | 5 | 1 | 20 |
| Phase 2 | 40 mg | 28 | 8 | 28.6 |

Combining the data from studies, the percent of patients with Grade 3 or higher hepatic adverse events supports the dose relationship with the 40 mg dose in combination with nivolumab exhibiting a 32.5% (13/40) rate while 30 mg in combination with nivolumab resulted in a 11.8% rate (2/17).

This significant decrease in the rate of meaningful hepatic adverse events supports dosing of 30 mg daily.

From the PK perspective, the Cₘₐₓ and AUC_{0-24 hrs} of eganelisib following 30 mg QD dosing with combination therapies (i.e., with nivolumab 240 mg Q2W or 480 mg Q4W) was 1.68 μg/mL and 29.2 g×hr/mL, respectively, on C2D1 (the table below, based on model-predicted geometric mean using data from 3 eganelisib clinical studies). Cₘₐₓ and AUC_{0-24 hrs} were 31% to 38% lower than those following 40 mg QD dosing. Additionally, the observed highest individual concentration value of eganelisib across all 3 studies was 61% lower for 30 mg QD versus 40 mg QD (4.42 and 11.4 μg/mL, respectively) based on the PK data cutoff of June-July 2020.

Model-Predicted IPI-549 Exposures on C1D1 and C2D1 for
30 mg and 40 mg QD Dosing With Combination Therapies

| Dose | Visit | Parameter | N | Mean | SD | Min | Median | Max | GeoMean | GeoCV (%) | GMR |
|---|---|---|---|---|---|---|---|---|---|---|---|
| 30 | C1D1 | Cₘₐₓ | 27 | 1.02 | 0.245 | 0.637 | 0.978 | 1.75 | 0.993 | 23.4 | 0.641 |
| mg | C1D1 | AUC_{0-24 hrs} | 27 | 16.7 | 4.20 | 7.90 | 15.9 | 26.3 | 16.1 | 26.6 | 0.617 |
| | C2D1 | Cₘₐₓ | 32 | 1.86 | 0.875 | 0.684 | 1.53 | 4.07 | 1.68 | 48.4 | 0.681 |
| | C2D1 | AUC_{0-24 hrs} | 32 | 33.6 | 18.9 | 8.18 | 25.8 | 83.3 | 29.2 | 58.1 | 0.693 |
| 40 | C1D1 | Cₘₐₓ | 159 | 1.65 | 0.565 | 0.346 | 1.56 | 3.63 | 1.55 | 37.1 | 1.00 |
| mg | C1D1 | AUC_{0-24 hrs} | 159 | 28.4 | 11.2 | 4.90 | 26.0 | 69.5 | 26.2 | 44.6 | 1.00 |
| | C2D1 | Cₘₐₓ | 92 | 2.77 | 1.36 | 0.480 | 2.42 | 8.83 | 2.47 | 51.7 | 1.00 |
| | C2D1 | AUC_{0-24 hrs} | 92 | 49.6 | 29.9 | 5.03 | 41.4 | 178 | 42.1 | 65.0 | 1.00 |

From the PD perspective, in the monotherapy dose escalation portion of the phase 1 trial, inhibition of pAKT (Thr308) following CXCL12 stimulated PI3K-γ pathway activation in monocytes using the whole blood PD assay demonstrated suppression of PI3K-γ pathway throughout the duration of the eganelisib dosing interval for all doses tested, including 30 mg. From the PK/PD perspective, eganelisib concentrations following 30 mg QD monotherapy were above the IC₉₀ value for PI3K-γ pathway suppression in monocytes (on-target effects) and well below the IC₅₀ value for PI3K-δ pathway suppression in B cells (off-target effects) on C2D1 in the phase 1 trial. Furthermore, pharmacodynamic changes indicative of enhanced immune activation including increased T cell reinvigoration and increased INF-γ-responsive cytokines CXCL9 and CXCL10 were observed to a similar extent at both the 30 mg and 40 mg doses.

Taken together, the above results indicate that eganelisib 30 mg QD is effective at inhibiting PI3K-γ without significant Grade 3 or higher hepatic adverse events.

### Example 6: A Phase 2, Multicenter, Randomized, Double-Blind, Placebo-Controlled Study to Evaluate the Efficacy and Safety of Eganelisib Administered in Combination with Nivolumab Compared to Nivolumab Monotherapy in the Treatment of PD-L1 Negative Patients with Immune Therapy-Naïve, Advanced Urothelial Carcinoma

### Study Population and Planned Number of Patients:

Patients with histologically or cytologically confirmed, programmed death ligand 1 (PD-L1) negative (tumor proportion score [TPS]<1%), urothelial carcinoma who progressed on or following at least 1 platinum-based chemotherapy regimen for the treatment of metastatic (Stage IV) or locally advanced surgically unresectable disease, or who had disease recurrence within 1 year of completing a platinum-based neoadjuvant or adjuvant therapy. Patients cannot have received prior anticancer immunotherapy, including anti-programmed cell death protein 1 and anti-PD-L1 antibody therapy, prior to randomization.

Up to approximately 216 patients are randomized with a 1:1 allocation ratio to receive treatment with nivolumab administered in combination with eganelisib (approximately 108) or nivolumab administered in combination with placebo (approximately 108). Randomization is stratified by presence or absence of liver metastases and geographic region.

### Objectives:

Primary Objective:
To compare progression-free survival (PFS) per Response Evaluation Criteria in Solid Tumors (RECIST) v1.1 as assessed by Independent Central Review of Response (ICRR) in PD-L1 negative patients following combination treatment with nivolumab plus eganelisib with that of nivolumab monotherapy.

Secondary Objectives:
To compare overall survival (OS) of PD-L1 negative patients following combination treatment with nivolumab plus eganelisib with that of nivolumab monotherapy.
To compare objective response rate (ORR) per RECIST v1.1 and immune RECIST (iRECIST) in PD-L1 negative patients following combination treatment with nivolumab plus eganelisib with that of nivolumab monotherapy.
To compare duration of response (DOR) in PD-L1 negative patients following combination treatment with nivolumab plus eganelisib with that of nivolumab monotherapy.
To compare PFS per iRECIST in PD-L1 negative patients following combination treatment with nivolumab plus eganelisib with that of nivolumab monotherapy.
To evaluate the safety of combination treatment with nivolumab plus eganelisib.

Exploratory Objectives:
To evaluate the pharmacokinetics (PK) of eganelisib administered in combination with nivolumab.
To characterize eganelisib exposure-response relationships for selected efficacy and safety endpoints.

### Study Endpoints:

Primary Endpoint:
PFS per RECIST v1.1 by ICRR, defined as the time from the date of randomization to the date of documented disease progression or death due to any cause.

Secondary Endpoints:
OS, defined as the time from the date of randomization to the date of death from any cause.
ORR, with objective response defined as best response of complete response (CR) or partial response (PR), as determined by RECIST v1.1 and iRECIST.
DOR, defined as the time from the first objective response (CR or PR) to documented disease progression in patients with CR or PR.
PFS per iRECIST
Incidence of treatment-emergent adverse events (TEAEs); hepatic TEAEs; immune-mediated adverse events (IMAEs); serious adverse events (SAEs), including deaths; adverse events (AEs) leading to treatment discontinuation; and changes from baseline in safety laboratory parameters, vital signs, and electrocardiograms (ECGs).

Exploratory Endpoints:
Population PK estimates, including inter- and intra-patient variability, covariate effects, and drug-interaction parameters.
Exposure-response parameters for select efficacy and safety endpoints.
Nivolumab exposure parameters and incidence of anti-nivolumab antibodies.

### Inclusion Criteria

All patients must meet the following criteria for inclusion:
1. ≥18 years of age.
2. Have signed and dated an independent review board-/independent ethics committee-approved informed consent form in accordance with regulatory and institutional guidelines. This must be obtained before the performance of any protocol-related procedures that are not part of normal patient care.
3. Willing and able to comply with scheduled visits, treatment schedule, laboratory tests, fresh tumor biopsies, and all other protocol requirements.
4. Patients with histologically or cytologically confirmed urothelial carcinoma (including mixed histologies of urothelial carcinoma with elements of other subtypes) of the renal pelvis, ureter, bladder, or urethra, who meet one of the following:
   a. Have progression or refractory disease. Patients must have had at least 1 platinum-based chemotherapy regimen for the treatment of metastatic (Stage IV) or locally advanced unresectable disease; or
   b. Have disease recurrence within 1 year of completing a platinum-based neoadjuvant or adjuvant therapy; and
   c. Have been treated or been ineligible for treatment with a fibroblast growth factor receptor (FGFR) inhibitor if the patient has known FGFR3 or FGFR2 genetic alterations.
5. At least 1 measurable disease lesion by computed tomography (CT) or magnetic resonance imaging (MRI) as defined by RECIST v1.1 performed within 30 days prior to the first dose of study drug.
6. Patients with PD-L1 negative (TPS<1%) test results confirmed by central laboratory (Dako PD-L1 immunohistochemical 28-8 pharmDx kit).
   a. Tumor material from core biopsies done before the screening period is acceptable if the biopsy was performed within 12 months and slides were cut <3 months prior to the planned treatment start and no new systemic cancer therapy was administered after the biopsy and before study entry. If no archival tissue is available, a baseline tumor biopsy is required.
   b. Willing to undergo 1 pre-treatment core biopsy (unless archival tumor tissue is available).
7. Eastern Cooperative Oncology Group (ECOG) performance status≤1.
8. Baseline laboratory values must meet the following criteria within 14 days of the first dose:
   a. Adequate hematologic function, defined as white blood cell count≥2.0×10⁹/L, absolute neutrophil count≥1.5×10⁹/L, hemoglobin≥9.0 g/dL, and platelet count≥100×10⁹/L.
   b. Creatinine clearance≥30 mL/min, as determined by either of the following:
      i. Estimation as calculated by Cockcroft-Gault equation;
      ii. Direct measurement by 24-hour urine collection.
   c. Aspartate aminotransferase and alanine aminotransferase<3×upper limit of normal (ULN).
   d. Total bilirubin≤1.5×ULN (unless elevated due to Gilbert's syndrome who can have total bilirubin<3.0 mg/dL).
   e. Albumin≥3 g/dL.
9. Patients who have received more than 2 prior lines of chemotherapy must not have liver metastases. Sequential chemotherapy given as a planned sequence to optimize response counts as 1 regimen.
10. Prior focal radiotherapy to an isolated bony or soft tissue metastasis should be completed at least 2 weeks before study drug administration.
11. All toxicities attributed to prior anticancer therapy, with the exception of neuropathy, alopecia, and fatigue, must have resolved to Grade 1 (per National Cancer Institute Common Terminology Criteria for Adverse Events [NCI CTCAE] version 5.0) or baseline before administration of study drug. Patients with toxicities attributed to prior anticancer therapy that are not expected to resolve and result in long-lasting sequelae, such as neuropathy after platinum-based therapy, are permitted to enroll. Neuropathy must have resolved to Grade 2 (per NCI CTCAE version 5.0).
12. Women of childbearing potential (WOCBP) must have a negative serum or urine β human chorionic gonadotropin (βhCG) pregnancy test (minimum sensitivity 25 IU/L or equivalent units of hCG) within 1 week before administration of study drug. WOCBP is defined as any female who has experienced menarche and who has not undergone surgical sterilization (hysterectomy or bilateral oophorectomy) and is not postmenopausal. Menopause is defined as 12 months of amenorrhea in a woman over age 45 years in the absence of other biological or physiological causes. In addition, women under the age of 55 years who have not undergone surgical sterilization must have a serum follicle stimulating hormone level>40 mIU/mL to confirm menopause. (Recommendations related to contraception and pregnancy testing in clinical trials https://www.uni-due.de/imperia/md/content/ethikkomission/kontrazeption.pdf).
13. Women must not be breastfeeding.
14. Willingness of male and female patients who are not surgically sterile or postmenopausal to use medically acceptable methods of birth control for the duration of the study treatment, including 30 days after the last dose of eganelisib and 5 months for females and 7 months for males after the last dose of nivolumab. At a minimum, patients must agree to use at least 1 highly effective method of contraception.
15. Azoospermic males and WOCBP who are continuously not heterosexually active are exempt from contraceptive requirements. However, WOCBP must still undergo pregnancy testing per protocol.

### Exclusion Criteria

Patients are to be excluded from the study if they meet any of the following criteria:
1. Active brain metastases or leptomeningeal metastases. Patients with brain metastases are eligible if these have been treated and there is no MRI evidence of progression for at least 4 weeks after treatment is complete and within 28 days prior to the first dose of study drug administration. There must also be no requirement for immunosuppressive doses of systemic corticosteroids (>10 mg/day prednisone equivalents) for at least 2 weeks prior to study drug administration. Patients with incidental findings of asymptomatic brain metastases at screening may start study treatment without prior radiation treatment after discussion between the Infinity Medical Monitor or designee and Investigator.
2. Any serious or uncontrolled medical disorder that, in the opinion of the Investigator, may increase the risk associated with study participation or study drug administration, impair the ability of the patient to receive protocol therapy, or interfere with the interpretation of study results.
3. Other prior malignancy active within the previous 3 years except for local or organ-confined early stage cancer that has been definitively treated with curative intent, does not require ongoing treatment, has no evidence of residual active disease, and has a negligible risk of recurrence and is therefore unlikely to interfere with the primary and secondary endpoints of the study, including response rate and safety.
4. Active, known, or suspected autoimmune disease. Patients with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.
5. Condition requiring systemic treatment with either corticosteroids (>10 mg daily prednisone equivalents) or other immunosuppressive medications within 14 days of the first dose of study drug. Inhaled or topical steroids and adrenal replacement steroid doses are permitted in the absence of active autoimmune disease.
6. Prior therapy with experimental antitumor vaccines; any immune cell co-stimulation or checkpoint pathways, such as anti-PD-1, anti-PD-L1, anti-programmed death ligand 2, anti-CD137, or anti-cytotoxic T-lymphocyte-associated protein 4 antibody, including ipilimumab, or other medicines specifically targeting the T cell; or eganelisib.
7. Treatment with any chemotherapy, radiation therapy, biologics for cancer, or investigational therapy within 14 days or 5 half-lives, whichever is shorter, of first administration of study drug (patients with prior cytotoxic or investigational products<28 days prior to treatment might be eligible after discussion between Investigator and Infinity Medical Monitor or designee, if toxicities from the prior treatment have resolved to CTCAE Grade 1 level).
8. Treatment with botanical preparations (e.g., herbal supplements or traditional Chinese medicines) intended for general health support or to treat the disease under study within 2 weeks prior to randomization.
9. Major surgery within 4 weeks prior to randomization.
10. Positive test for hepatitis B virus using hepatitis B surface antigen and hepatitis B core antibody or positive test for hepatitis C virus (HCV) using HCV RNA test indicating acute or chronic infection.
11. Known history of testing positive for human immunodeficiency virus or known acquired immunodeficiency syndrome.
12. Dependence on continuous supplemental oxygen use.
13. History of allergy to study drug components or severe hypersensitivity reaction to any monoclonal antibody.
14. Ongoing systemic bacterial, fungal, or viral infections at screening. (NOTE: Patients on antimicrobial, antifungal, or antiviral prophylaxis are not specifically excluded if all other inclusion/exclusion criteria are met.)
15. Administration of a live or attenuated vaccine within 6 weeks of first dose of study drug.
16. Administration of any of the following within 1 week prior to the administration of study drug:
   a. Strong inhibitors or inducers of cytochrome P450 (CYP) 3A4 and CYP2C8, including grapefruit, grapefruit juice, and herbal supplements.
   b. P-glycoprotein (P-gp) inhibitors.
17. Baseline QT interval corrected with Fridericia's method (QTcF)>480 ms. (NOTE: Criterion does not apply to patients with a right or left bundle branch block.)
18. Prior surgery or gastrointestinal dysfunction that may affect drug absorption (e.g., gastric bypass surgery, gastrectomy).
19. WOCBP who are pregnant or breastfeeding.
20. Women with a positive pregnancy test at enrollment or prior to administration of study drug.
21. Past medical history of interstitial lung disease, drug-induced interstitial lung disease, radiation pneumonitis that required steroid treatment, or any evidence of clinically active interstitial lung disease.
22. History of stroke, unstable angina, myocardial infarction, or ventricular arrhythmia requiring medication or mechanical control within the last 6 months prior to screening.

### Study Design

The study is prospective, multicenter, randomized, double-blind, and placebo controlled in design. Following informed consent and determination of eligibility for the study, patients are randomized in a 1:1 ratio to receive either intravenous (IV) nivolumab 480 mg every 4 weeks (Q4W) in combination with oral (PO) eganelisib 30 mg once daily (QD) or IV nivolumab 480 mg Q4W in combination with placebo capsules administered PO QD. Randomization is stratified by presence or absence of liver metastasis and geographic region.

Screening procedures to determine patient eligibility for the study are conducted within 30 days of the first dose of study drug(s). Treatment cycles are 28 days in duration and patients attend clinic visits on Days 1 and 15 of Cycles 1 to 5 and Day 1 of every cycle thereafter until unacceptable toxicity, confirmed progression of disease, withdrawal of consent, or other treatment discontinuation criteria are met.

During the treatment period, patients are evaluated for safety based on monitoring for AEs, concomitant medications, and physical examinations, vital signs, ECGs, clinical laboratory tests (hematology, chemistry, urinalysis), and ECOG performance status. Blood for PK assessment are obtained through 6 hours following the eganelisib (or placebo) dose on Day 1 of Cycles 1 and 2.

Response to treatment is determined by the Investigators and central readers based on radiographic evaluations and assessments conducted using RECIST v1.1 and iRECIST during screening, every 8 (1) weeks through Week 48, and at least every 12 (±1) weeks thereafter until confirmed progression of disease (i.e., clinical deterioration or confirmed radiological progression [at least 4 weeks apart]). Response assessments are to be conducted as noted, independent of dose delays and/or dose interruptions. Patients who develop progressive disease (PD) per RECIST v1.1, are clinically stable, and provide consent may continue randomized treatment and be followed for improved response or confirmation of PD after discussion with sponsor medical monitor or designee.

Following discontinuation of study treatment, all patients attend 2 safety follow-up visits, conducted at 30 (±7) days and 100 (±10) days after the last dose of study treatment.

Patients who discontinue study treatment without confirmed progression of disease continue to undergo disease response assessments post-treatment as per protocol (i.e., every 8 or 12 weeks) until disease progression is confirmed, alternate anticancer therapy is initiated, or withdrawal of consent.

All patients are followed for survival and alternate anticancer therapy every 3 months through 2 years after the last day of study treatment or until 2 years after the enrollment of the last patient in the study, whichever occurs earlier.

### Study Drug(s), Dosage, Mode of Administration, and Treatment Duration

For patients randomized to receive nivolumab in combination with eganelisib, eganelisib are administered PO at a dose of 30 mg QD. The eganelisib drug product is formulated in 2 capsule strengths (5 and 30 mg). Patients randomized to the nivolumab monotherapy arm are administered placebo that are identical in appearance to the active treatment and are dosed in the same manner.

Nivolumab is administered by IV infusion over 30 (±5) minutes at a dose of 480 mg Q4W.

Eganelisib and/or nivolumab treatment may continue until either unacceptable toxicity, confirmed progression of disease, withdrawal of consent, or other withdrawal criteria are met. Under specific conditions, patients may continue treatment beyond RECIST v1.1 PD. The maximum duration of nivolumab therapy is 2 years.

### Statistical Methods

### Sample Size Determination:

The primary efficacy endpoint is PFS. A clinically meaningful improvement is defined as a 65% increase in median time to progression in the combination treatment arm (median=3.3 months) over the monotherapy arm (median=2.0 months). Assuming a 2-sided alpha of 0.05, power of 90%, and a randomization ratio of 1:1 between the combination treatment arm and the monotherapy arm, the study data are considered mature and the final analysis is performed when 172 events are observed. Assuming a censoring rate of 20%, approximately 216 patients are randomized and stratified by presence or absence of liver metastases and geographic region.

### Key Analysis Sets:

Modified Intent-to-Treat (ITT) Analysis Set: defined as all patients randomized into the study who receive any amount of study drug(s) with patients analyzed based on randomized treatment. This analysis set is the primary analysis set for all efficacy endpoints.

All-Treated Analysis Set: defined as all patients who receive any amount of study drug(s) with patients analyzed based on actual treatment received. This analysis set is the primary analysis set for all safety endpoints.

### Efficacy Analyses:

The primary efficacy analysis is the comparison of PFS between the treatment arms for patients in the ITT population using Log-rank test. Hazard ratio is estimated with Cox proportional hazard model including treatment and stratification factors as covariates.

All time to event endpoints, including PFS, DOR, and OS, are summarized using Kaplan-Meier methods, including the frequency and percent of patients censored and with events, and the estimated median and 25th and 75th percentiles with associated 95% confidence intervals. Note that the analysis of DOR is based on patients who achieve a best response of CR or PR. Date of progression used in time-to-event analyses is based on first documentation of PD, regardless of confirmation.

Objective response are summarized as the percentage of patients achieving a best overall response of CR or PR per RECIST v1.1. The treatment difference in ORR is tested using Cochran Mantel Haenszel test adjusting for stratification factors.

### Pharmacokinetic Pharmacodynamic Analyses:

PK parameters are determined using standard noncompartmental analysis methods. The PK parameters to be assessed include, but are not necessarily limited to: maximum observed plasma concentration, time of maximum observed plasma concentration, area under the plasma concentration-time curve from time zero to the last quantifiable time point, and accumulation upon multiple-dose administration. Additionally, population PK/pharmacodynamic analyses are conducted, including determination of population PK estimates, inter- and intra-patient variabilities, the impact of covariates, and characterization of exposure-response parameters for selected efficacy and safety endpoints. The data may be combined with data from other studies of eganelisib and the results are reported separately.

Nivolumab exposure parameters and incidence of anti-nivolumab antibodies are summarized.

### Safety Analyses:

AEs are coded using the Medical Dictionary for Regulatory Activities (MedDRA) version 23.0 or higher.

TEAEs, defined as an AE that emerges or worsens in the period from the first dose of study treatment to 100 days after the last dose of eganelisib or nivolumab (whichever is dosed last) or until starting another anticancer treatment, are summarized by treatment group and by the frequency of patients experiencing TEAEs corresponding to MedDRA system organ class and preferred term. Separate tabulations are produced for TEAEs assessed as related to study drug(s), TEAEs≥Grade 3 in severity, hepatic TEAEs, IMAEs, TEAEs leading to death, treatment-emergent SAEs, SAEs related to study drug(s), and TEAEs that led to dose interruption, dose reduction, and/or treatment discontinuation.

Descriptive statistics for actual values and changes from baseline to each visit and to last on study evaluation are summarized by treatment group for safety laboratory parameters, vital signs, and electrocardiogram (ECG) parameters. Shifts in NCI CTCAE grade from baseline to the maximum post-baseline grade and to the grade at the last on study evaluation are summarized by treatment group for applicable laboratory data.

Concomitant medications are coded using the World Health Organization Drug Dictionary. Results are tabulated by anatomic therapeutic class and preferred term.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims. Various publications, patents and patent applications are cited herein, the disclosures of which are incorporated by reference in their entireties.

What is claimed is:

1. A method for treating a cancer in a subject, comprising administering to the subject a therapeutically effective amount of a compound of the formula: 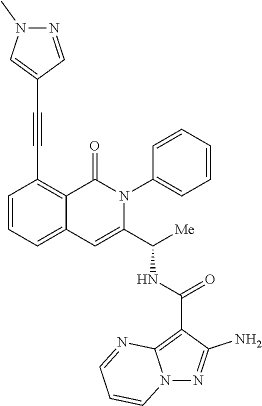 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, and wherein the cancer is PD-L1 negative.

2. A method for treating a cancer in a subject, comprising: (i) identifying the cancer in the subject to be PD-L1 negative, and (ii) administering to the subject a therapeutically effective amount of a compound of the formula: 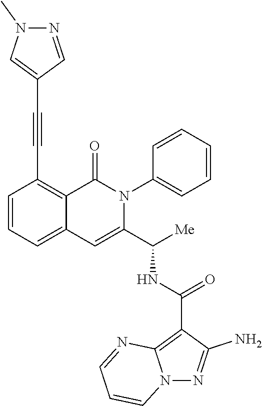 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof.

3. The method of claim 1 or 2, wherein the cancer is selected from one or more of: a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an esophageal cancer, a hepatocellular cancer, a gastric cancer, a gastrointestinal stromal tumor (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumor, diffuse type giant cell tumor, and glioblastoma.

4. The method of claim 1 or 2, wherein the cancer is a solid tumor.

5. The method of claim 4, wherein the solid tumor is melanoma, lung cancer, head and neck cancer, renal cell carcinoma, gallbladder carcinoma, breast cancer, colon cancer, glioblastoma, adrenocortical carcinoma, mesothelioma, colorectal cancer, ovarian cancer, endometrial cancer, or urothelial carcinoma.

6. The method of claim 5, wherein the solid tumor is breast cancer.

7. The method of claim 6, wherein the breast cancer is triple negative breast cancer.

8. The method of claim 5, wherein the solid tumor is head and neck cancer.

9. The method of claim 8, wherein the head and neck cancer is head and neck squamous cell carcinoma.

10. The method of claim 5, wherein the solid tumor is lung cancer.

11. The method of claim 10, wherein the lung cancer is non-small cell lung cancer.

12. The method of claim 5, wherein the solid tumor is melanoma.

13. The method of claim 5, wherein the solid tumor is colon cancer.

14. The method of claim 5, wherein the solid tumor is glioblastoma.

15. The method of claim 5, wherein the solid tumor is renal cell carcinoma.

16. The method of claim 15, wherein the renal cell carcinoma is clear cell renal cell carcinoma.

17. The method of claim 5, wherein the solid tumor is gallbladder carcinoma.

18. The method of claim 17, wherein the gallbladder carcinoma is microsatellite-stable gallbladder carcinoma.

19. The method of claim 5, wherein the solid tumor is adrenocortical carcinoma.

20. The method of claim 5, wherein the solid tumor is mesothelioma.

21. The method of claim 20, wherein the mesothelioma is epithelioid mesothelioma, sarcomatoid mesothelioma, or biphasic mesothelioma.

22. The method of claim 5, wherein the solid tumor is colorectal cancer.

23. The method of claim 5, wherein the solid tumor is ovarian cancer.

24. The method of claim 5, wherein the solid tumor is endometrial cancer.

25. The method of claim 5, wherein the solid tumor is urothelial carcinoma.

26. The method of claim 1 or 2, wherein the cancer is a hematological cancer.

27. The method of claim 26, wherein the hematological cancer is leukemia or lymphoma.

28. The method of claim 26, wherein the hematological cancer is acute lymphocytic leukemia (ALL), chronic lymphocytic leukemia (CLL), prolymphocytic leukemia (PLL), hairy cell leukemia (HLL), Waldenstrom's macroglobulinemia (WM), peripheral T cell lymphomas (PTCL), adult T cell leukemia/lymphoma (ATLL), cutaneous T-cell lymphoma (CTCL), large granular lymphocytic leukemia (LGL), acute myelocytic leukemia (AML), Hodgkin lymphoma (HL), non-Hodgkin lymphoma (NHL), follicular lymphoma, diffuse large B-cell lymphoma (DLBCL), mantle cell lymphoma (MCL), mastocytosis, multiple myeloma (MM), myelodysplastic syndrome (MDS), or myeloproliferative disorder (MPD).

29. The method of any one of claims 1 to 28, wherein the subject has high-circulating myeloid-derived suppressor cells.

30. The method of any one of claims 1 to 29, wherein the cancer is locally advanced and/or metastatic.

31. The method of any one of claims 1 to 30, further comprising administering to the subject a therapeutically effective amount of a second agent.

32. The method of claim 31, wherein the second agent is an immune checkpoint therapy.

33. The method of claim 32, wherein the immune checkpoint therapy is a PD-L1 inhibitor.

34. The method of claim 33, wherein the PD-L1 inhibitor is atezolizumab, YW243.55.S70, MSB0010718C, MDX-1105, or MEDI-4736.

35. The method of claim 34, wherein the PD-L1 inhibitor is atezolizumab.

36. The method of claim 32, wherein the immune checkpoint therapy is a PD-1 inhibitor.

37. The method of claim 36, wherein the PD-1 inhibitor is nivolumab, pembrolizumab, pidilizumab, AMP-244, or AMP-514.

38. The method of claim 37, wherein the PD-1 inhibitor is nivolumab.

39. The method of any one of claims 1 to 38, further comprising administering to the subject a therapeutically effective amount of a third agent.

40. The method of claim 39, wherein the third agent is nab-paclitaxel.

41. The method of claim 39, wherein the third agent is bevacizumab.

42. A method for treating breast cancer in a subject, comprising administering to the subject a therapeutically effective amount of a compound of the formula: 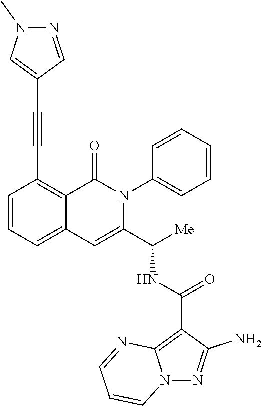 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-L1 inhibitor, wherein the breast cancer is PD-L1 negative.

43. A method for treating breast cancer in a subject, comprising: (i) identifying the breast cancer in the subject to be PD-L1 negative, and (ii) administering to the subject a therapeutically effective amount of a compound of the formula: 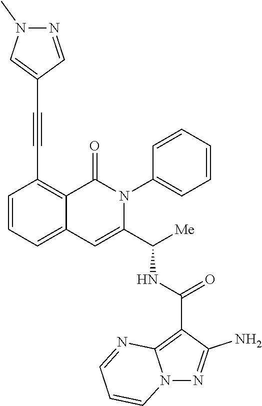 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-L1 inhibitor.

44. The method of claim 42 or 43, wherein the breast cancer is triple negative breast cancer.

45. The method of claim 44, wherein the breast cancer is unresectable locally advanced or metastatic triple negative breast cancer.

46. The method of any one of claims 42 to 45, wherein the PD-L1 inhibitor is atezolizumab.

47. The method of claim 46, wherein atezolizumab is administered intravenously at a dose of about 840 mg on days 1 and 15 of one or more 28-day cycles.

48. The method of any one of claims 42 to 47, wherein the administration of the compound and the PD-L1 inhibitor is further in combination with nab-paclitaxel.

49. The method of claim 48, wherein nab-paclitaxel is administered intravenously at a dose of about 100 mg/m² on days 1, 8, and 15 of one or more 28-day cycles.

50. The method of any one of claims 42 to 49, wherein the method is for treating breast cancer as front-line treatment.

51. A method for treating renal cell carcinoma in a subject, comprising: administering to the subject a therapeutically effective amount of a compound of the formula: 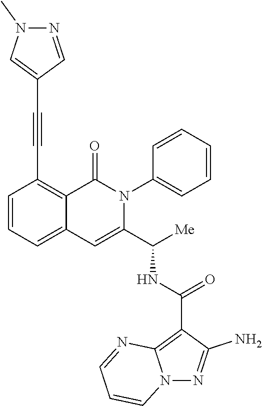 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-L1 inhibitor, wherein the renal cell carcinoma is PD-L1 negative.

52. A method for treating renal cell carcinoma in a subject, comprising: (i) identifying the renal cell carcinoma in the subject to be PD-L1 negative, and (ii) administering to the subject a therapeutically effective amount of a compound of the formula: 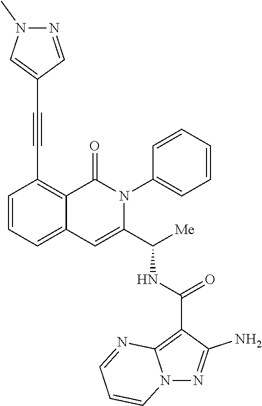 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-L1 inhibitor.

53. The method of claim 51 or 52, wherein the renal cell carcinoma is clear cell renal cell carcinoma.

54. The method of any one of claims 51 to 53, wherein the renal cell carcinoma is locally advanced and/or metastatic.

55. The method of any one of claims 51 to 54, wherein the PD-L1 inhibitor is atezolizumab.

56. The method of claim 55, wherein atezolizumab is administered intravenously at a dose of about 1200 mg on day 1 of one or more 21-day cycles.

57. The method of any one of claims 51 to 56, wherein the administration of the compound and the PD-L1 inhibitor is further in combination with bevacizumab.

58. The method of claim 57, wherein bevacizumab is administered intravenously at a dose of about 15 mg/kg on day 1 of one or more 21-day cycles.

59. The method of any one of claims 51 to 58, wherein the method is for treating renal cell carcinoma as front-line treatment.

60. A method for treating urothelial carcinoma in a subject, comprising: administering to the subject a therapeutically effective amount of a compound of the formula: 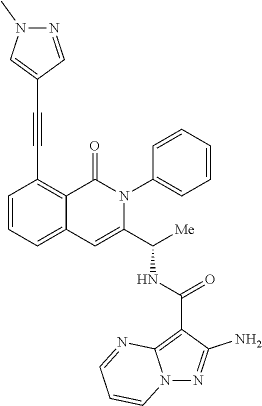 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-1 inhibitor or a PD-L1 inhibitor, wherein the urothelial carcinoma is PD-L1 negative.

61. A method for treating urothelial carcinoma in a subject, comprising: (i) identifying the urothelial carcinoma in the subject to be PD-L1 negative, and (ii) administering to the subject a therapeutically effective amount of a compound of the formula: 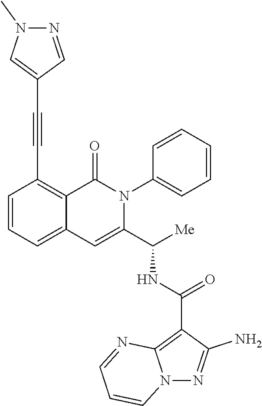 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with a PD-1 inhibitor or a PD-L1 inhibitor.

62. The method of claim 60 or 61, wherein the urothelial carcinoma is advanced urothelial carcinoma.

63. The method of claim 60 or 61, wherein the urothelial carcinoma is metastatic urothelial carcinoma.

64. The method of any one of claims 60 to 63, wherein the subject is naïve to immune checkpoint therapy.

65. The method of any one of claims 60 to 64, wherein the urothelial carcinoma has progressed or recurred following treatment with platinum-based chemotherapy.

66. The method of any one of claims 60 to 65, wherein the administration of the compound is in combination with a PD-1 inhibitor.

67. The method of claim 66, wherein the PD-1 inhibitor is nivolumab.

68. The method of claim 67, wherein nivolumab is administered intravenously at a dose of about 480 mg once per 4 weeks (Q4W).

69. The method of any one of claims 60 to 64, wherein the administration of the compound is in combination with a PD-L1 inhibitor.

70. The method of claim 69, wherein the PD-L1 inhibitor is atezolizumab.

71. The method of any one of claims 1 to 70, wherein the compound, or a pharmaceutically acceptable salt thereof, is administered at a dose of about 10 to about 60 mg once daily.

72. The method of claim 71, wherein the compound, or a pharmaceutically acceptable salt thereof, is administered at a dose of about 20, about 30, or about 40 mg once daily.

73. The method of claim 72, wherein the compound, or a pharmaceutically acceptable salt thereof, is administered at a dose of about 30 mg once daily.

74. A method for treating a PD-L1 negative patient with immune therapy-naïve, advanced urothelial carcinoma, comprising administering to the patient a therapeutically effective amount of a compound of the formula: 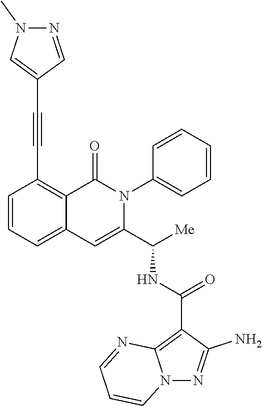 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with nivolumab.

75. A method for treating a PD-L1 negative patient with immune therapy-naïve, advanced urothelial carcinoma, comprising: (i) identifying the patient to be PD-L1 negative, and (ii) administering to the patient a therapeutically effective amount of a compound of the formula: 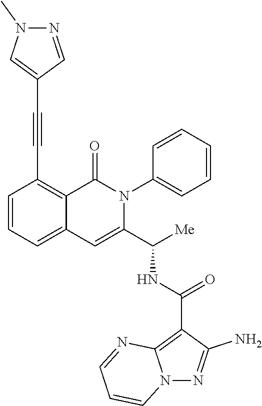 [SMILES: C[C@H](NC(=O)c1c(N)nn2cccnc12)c1cc2cccc(C#Cc3cnn(C)c3)c2c(=O)n1-c1ccccc1]
or a pharmaceutically acceptable salt thereof, in combination with nivolumab.

76. The method of claim 74 or 75, wherein the compound is administered orally at a dose of about 30 mg once daily, and nivolumab is administered intravenously at a dose of about 480 mg once per 4 weeks (Q4W).

77. The method of claim 76, wherein nivolumab is administered by IV infusion over 30±5 minutes.

78. The method of any one of claims 1 to 77, wherein the compound, or a pharmaceutically acceptable salt thereof, is administered orally.

79. The method of any one of claims 1 to 78, wherein free base of the compound is administered.

80. The method of any one of claims 1 to 79, wherein the treatment results in Grade 3 or higher hepatic adverse events in no more than 20% of the subjects (patients).

81. The method of any one of claims 1 to 80, wherein the administration of the compound results in an observed maximum plasma concentration (Cₘₐₓ) of the compound of no more than 5 μg/mL.

82. The method of any one of claims 1 to 80, wherein the administration of the compound results in a geometric mean observed maximum plasma concentration (Cₘₐₓ) of the compound of no more than 2 g/mL.

83. The method of any one of claims 1 to 82, wherein the administration of the compound results in an area under the concentration time curve (AUC₀₋₂₄) of the compound of no more than 100 μg×hr/mL.

84. The method of any one of claims 1 to 82, wherein the administration of the compound results in a geometric mean area under the concentration time curve (AUC₀₋₂₄) of the compound of no more than 35 g×hr/mL.

85. The method of any one of claims 81 to 84, wherein the compound is administered in 28-day cycles and the Cₘₓ or AUC₀₋₂₄ are measured around Cycle 2 Day 1.

86. The method of any one of claims 1 to 85, wherein the treatment results in an increase in progression free survival (PFS), overall survival (OS), overall response rate (ORR), complete response (CR), partial response (PR), or duration of response (DOR).

87. The method of any one of claims 1 to 86, wherein the treatment results in increased immune activation.

88. The method of claim 87, wherein the increased immune activation comprises increased T cell reinvigoration.

89. The method of any one of claims 1 to 88, wherein the treatment results in decreased immune suppression.

90. The method of claim 89, wherein the decreased immune suppression comprises decreased MDSC.
